(19) United States
(12) Patent Application Publication
(10) Pub. No.: US 2010/0144606 A1
(43) Pub. Date: Jun. 10, 2010

(54) COMBINATION 408

(75) Inventors: Stephen Connolly, Loughborough (GB); Adrian Fisher, Loughborough (GB); Alexander Humphries, Loughborough (GB); Andrew Watts, Loughborough (GB); Katherine Wiley, Loughborough (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/488,027

(22) Filed: Jun. 19, 2009

Related U.S. Application Data
(60) Provisional application No. 61/074,183, filed on Jun. 20, 2008.

(51) Int. Cl. A61K 38/12; C07C 229/28; A61P 11/06

(52) U.S. Cl. 514/9; 560/38

(57) ABSTRACT

The invention provides a pharmaceutical product, kit or composition comprising a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof, and a second active ingredient selected from: a non-steroidal Glucocorticoid Receptor (GR Receptor) Agonist; an antioxidant; a CCR1 antagonist; a chemokine antagonist (not CCR1); a corticosteroid; a CRTh2 antagonist; a DP1 antagonist; an Histone Deacetylase Inducer; an IKK2 inhibitor; a COX inhibitor; a lipoxygenase inhibitor; a leukotriene receptor antagonist; an MPO inhibitor; a muscarinic antagonist; a p38 inhibitor; a PDE inhibitor; a PPARγ agonist; a protease inhibitor; a Statin; a thromboxane antagonist; a vasodilator; or, an ENAC blocker (Epithelial Sodium-channel blocker); and its use in the treatment of respiratory disease (for example chronic obstructive pulmonary disease (COPD) or asthma); to certain salts of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide and to an intermediate useful in the manufacture of this pharmaceutically active substance and salts thereof.

This patent claims benefit under 35 USC §119(e) to U.S. Provisional Application No. 61/074,183 (filed 20 Jun. 2008). The entire text of U.S. Provisional Application No. 61/074,183 is incorporated by reference into this patent.

The present invention relates to a combination of two or more pharmaceutically active substances for use in the treatment of respiratory diseases (for example chronic obstructive pulmonary disease (COPD) or asthma), to certain salts of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide and to an intermediate useful in the manufacture of this pharmaceutically active substance and salts thereof.

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

Among the most common of the respiratory diseases is asthma. Asthma is generally defined as an inflammatory disorder of the airways with clinical symptoms arising from intermittent airflow obstruction. It is characterised clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells. The two most important conditions covered by COPD are chronic bronchitis and emphysema.

Chronic bronchitis is a long-standing inflammation of the bronchi which causes increased production of mucous and other changes. The patients' symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Therapeutic agents used in the treatment of respiratory diseases include corticosteroids. Corticosteroids (also known as glucocorticosteroids or glucocorticoids) are potent anti-inflammatory agents. Whilst their exact mechanism of action is not clear, the end result of corticosteroid treatment is a decrease in the number, activity and movement of inflammatory cells into the bronchial submucosa, leading to decreased airway responsiveness. Corticosteroids may also cause reduced shedding of bronchial epithelial lining, vascular permeability, and mucus secretion. Whilst corticosteroid treatment can yield important benefits, the efficacy of these agents is often far from satisfactory, particularly in COPD. Moreover, whilst the use of steroids may lead to therapeutic effects, it is desirable to be able to use steroids in low doses to minimise the occurrence and severity of undesirable side effects that may be associated with regular administration. Recent studies have also highlighted the problem of the acquisition of steroid resistance amongst patients suffering from respiratory diseases. For example, cigarette smokers with asthma have been found to be insensitive to short term inhaled corticosteroid therapy, but the disparity of the response between smokers and non-smokers appears to be reduced with high dose inhaled corticosteroid (Tomlinson et al., Thorax 2005; 60:282-287).

A further class of therapeutic agent used in the treatment of respiratory diseases are bronchodilators. Bronchodilators may be used to alleviate symptoms of respiratory diseases by relaxing the bronchial smooth muscles, reducing airway obstruction, reducing lung hyperinflation and decreasing shortness of breath. Types of bronchodilators in clinical use include β₂ adrenoceptor agonists, muscarinic receptor antagonists and methylxanthines. Bronchodilators are prescribed mainly for symptomatic relief and they are not considered to alter the natural history of respiratory diseases.

N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide and its di-D-mandelate, dihydrobromide and bis-trifluoroacetate salts are β adrenoceptor agonists and are disclosed in PCT/GB2007/004861. The compound and its salts show at least a 5-fold selectivity of β adrenoceptor agonism over adrenergic α1D, adrenergic β1 and dopamine D2 activities.

Combination products comprising a β₂ adrenoceptor agonist and a corticosteroid are available. One such product is a combination of budesonide and formoterol fumarate (marketed by AstraZeneca under the tradename Symbicort®), which has proven to be effective in controlling asthma and COPD, and improving quality of life in many patients.

In view of the complexity of respiratory diseases such as asthma and COPD, it is unlikely that any one mediator can satisfactorily treat a respiratory disease alone. Moreover, whilst combination treatments using a β₂ adrenoceptor agonist and a corticosteroid deliver significant patient benefits, there remains a medical need for new therapies against respiratory diseases such as asthma and COPD, in particular for therapies with disease modifying potential.

Accordingly, the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof, and a second active ingredient selected from:

a non-steroidal Glucocorticoid Receptor (GR Receptor) Agonist;

an antioxidant;

a CCR1 antagonist;

a chemokine antagonist (not CCR1);

a corticosteroid;

a CRTh2 antagonist;

a DP1 antagonist;

an Histone Deacetylase Inducer;

an IKK2 inhibitor;

a COX inhibitor;

a lipoxygenase inhibitor;

a leukotriene receptor antagonist;

an MPO inhibitor;

a muscarinic antagonist;

a p38 inhibitor;

a PDE inhibitor;

a PPARγ agonist;

a protease inhibitor;

a Statin;

a thromboxane antagonist;

a vasodilator; or,

an ENAC blocker (Epithelial Sodium-channel blocker); provided that the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or,
(R)-1-[(2-Methyl-pyridin-4-ylcarbamoyl)-methyl]-3-(1-phenyl-cycloheptanecarbonyloxy)-1-azonia-bicyclo[2.2.2]octane X;

wherein X represents a pharmaceutically acceptable anion of a mono or polyvalent acid.

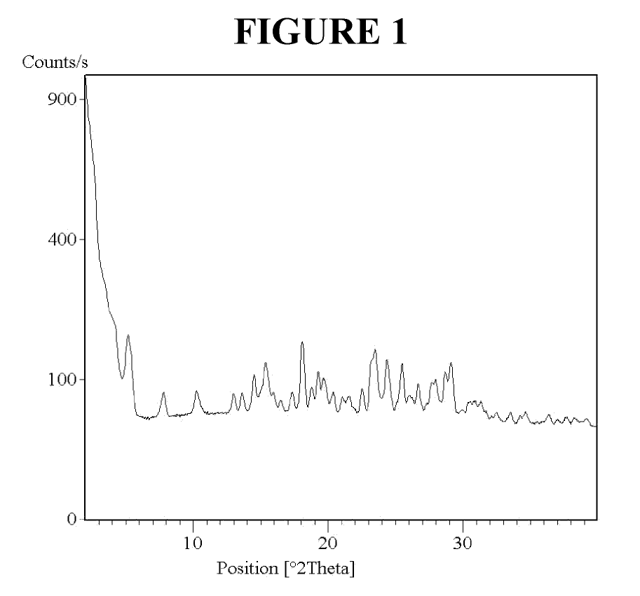

FIG. 1 shows the XRPD spectra for Form C of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form C).

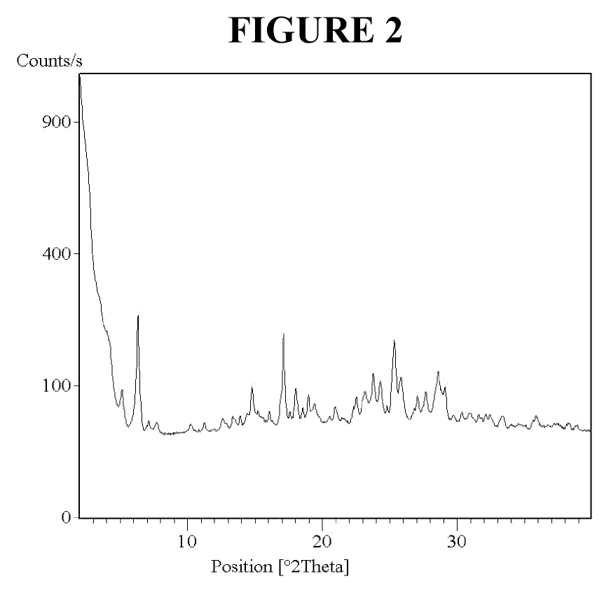

FIG. 2 shows the XRPD spectra for Form D of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form D).

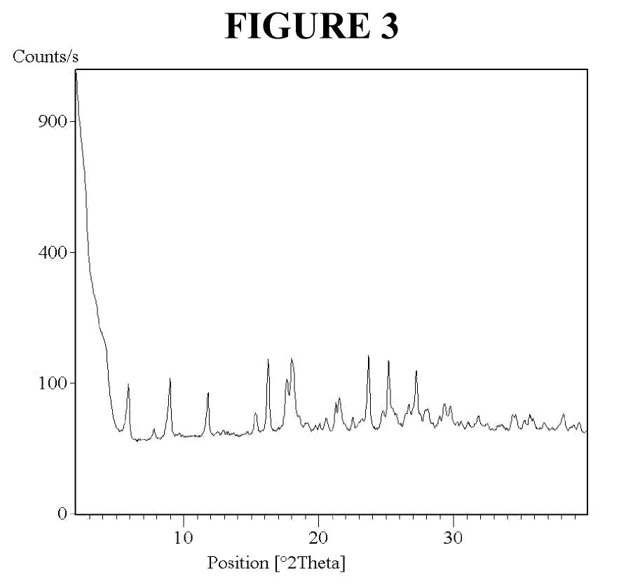

FIG. 3 shows the XRPD spectra for Form E of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form E).

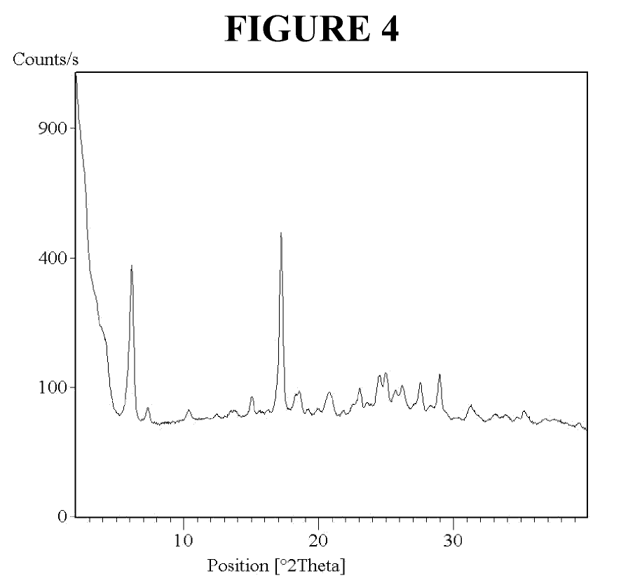

FIG. 4 shows the XRPD spectra for Form F of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form F).

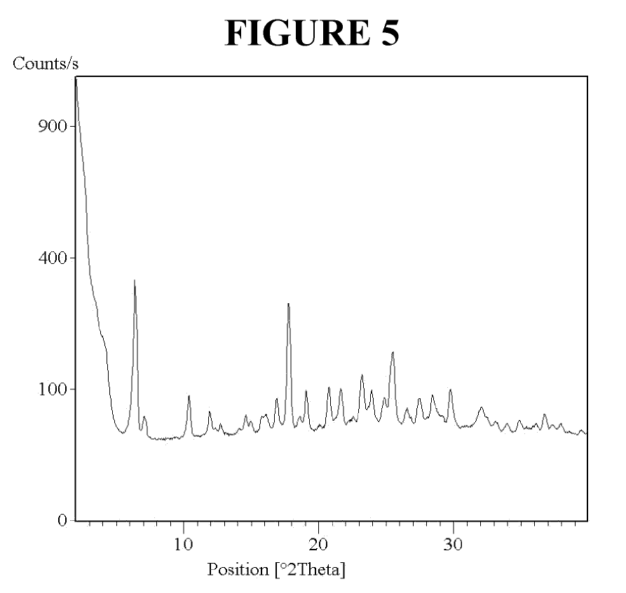

FIG. 5 shows the XRPD spectra for Form G of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form G).

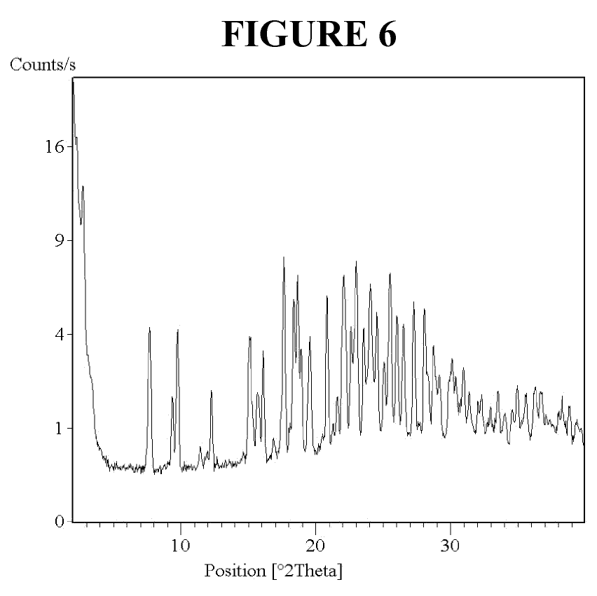

FIG. 6 shows the XRPD spectra for Form H of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Preparation 2, Form H).

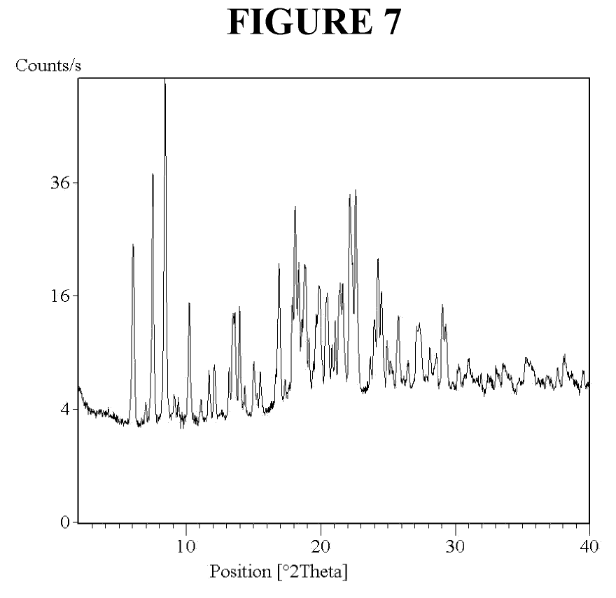

FIG. 7 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt (Preparation 3, Form A).

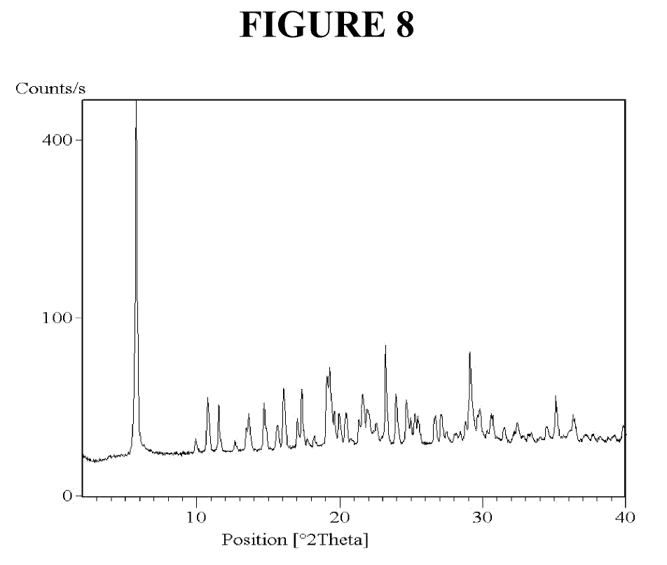

FIG. 8 shows the XRPD spectra for Form A of (R)-1-[2-(4-fluoro-phenyl)-ethyl]-3-((S)-2-phenyl-2-piperidin-1-yl-propionyloxy)-1-azonia-bicyclo[2.2.2]octane bromide (Preparation 4, Form A).

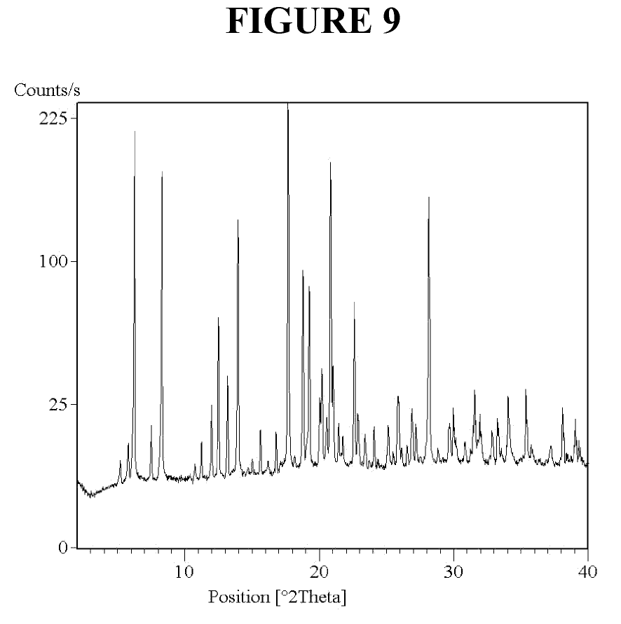

FIG. 9 shows the XRPD spectra for Form C of (R)-1-[2-(4-fluoro-phenyl)-ethyl]-3-((S)-2-phenyl-2-piperidin-1-yl-propionyloxy)-1-azonia-bicyclo[2.2.2]octane bromide (Preparation 4, Form C).

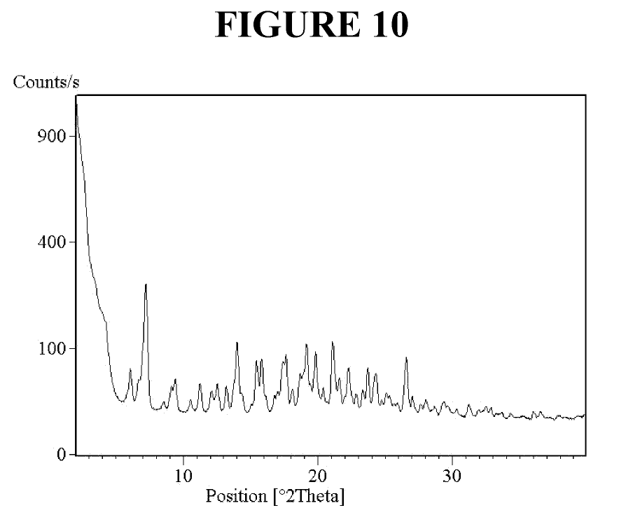

FIG. 10 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide (Preparation 5, Form A).

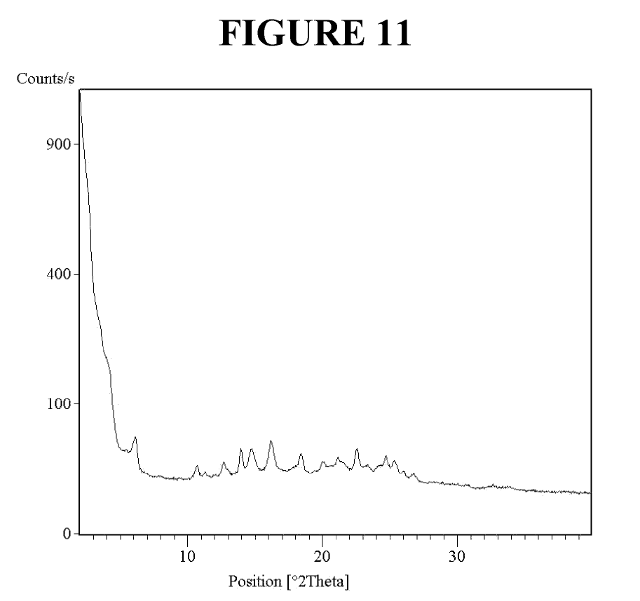

FIG. 11 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Preparation 6, Form A).

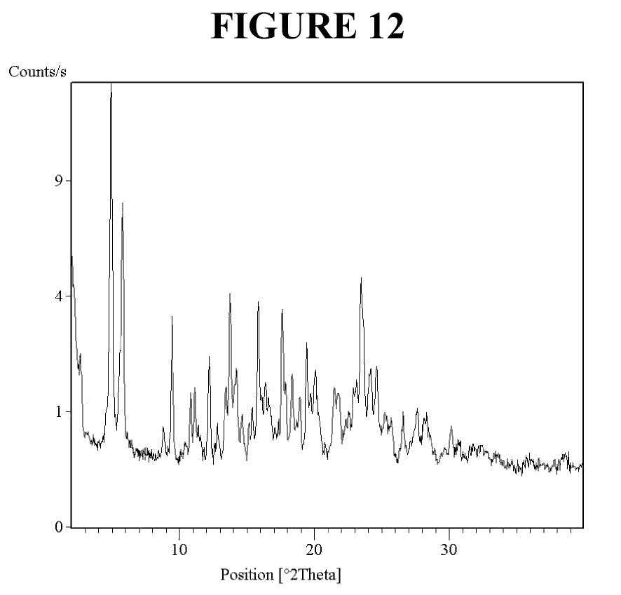

FIG. 12 shows the XRPD spectra for Form B of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Preparation 6, Form B).

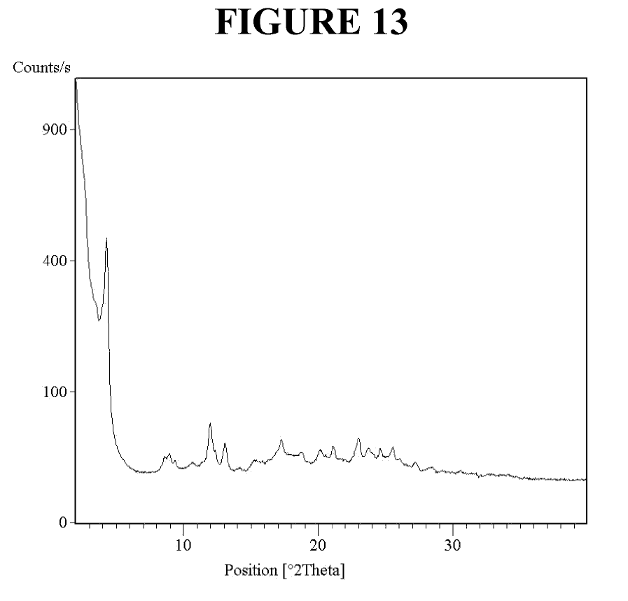

FIG. 13 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihippuric acid salt (Preparation 7, Form A).

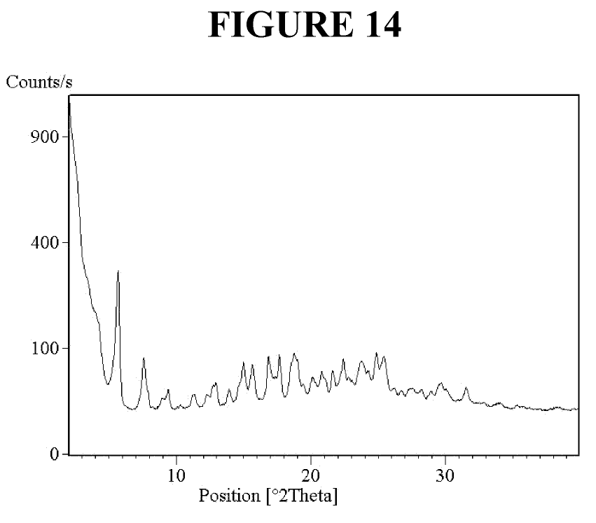

FIG. 14 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide sulfuric acid salt (Preparation 8, Form A).

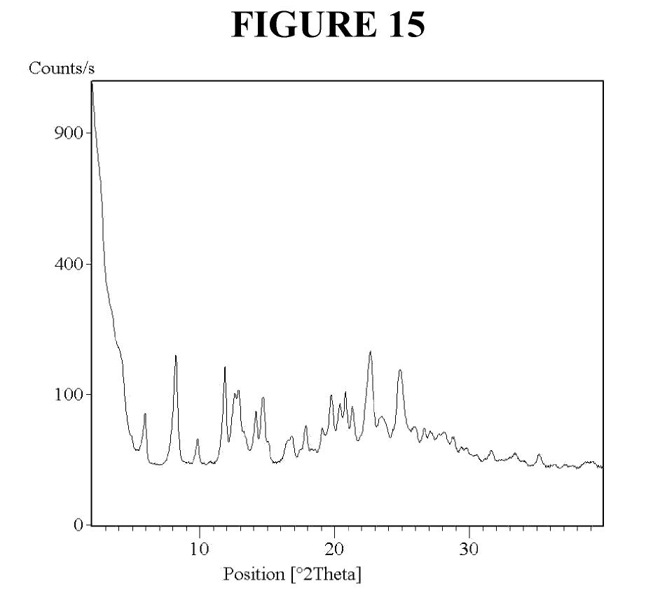

FIG. 15 shows the XRPD spectra for Form A of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Preparation 9, Form A).

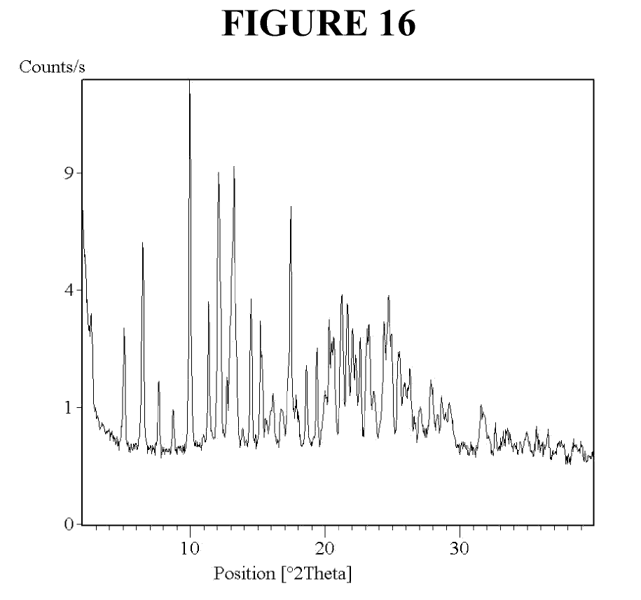

FIG. 16 shows the XRPD spectra for Form B of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Preparation 9, Form B).

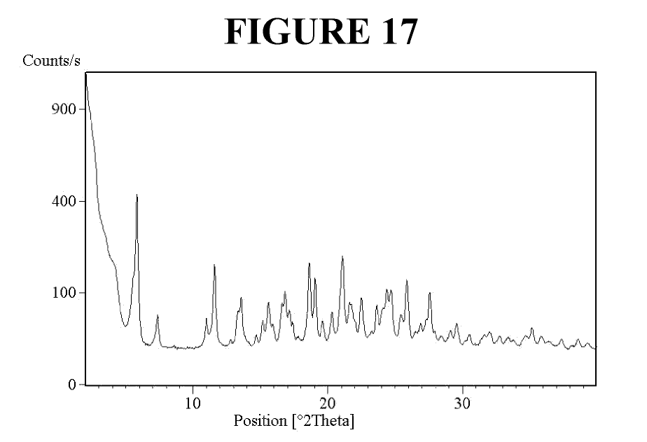

FIG. 17 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form A).

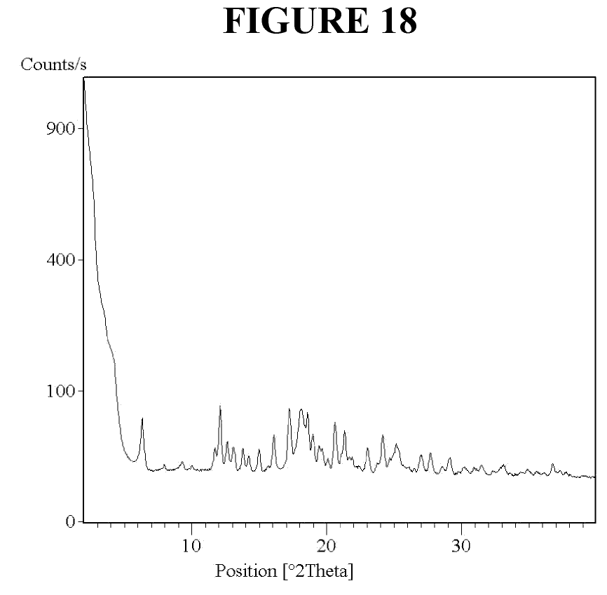

FIG. 18 shows the XRPD spectra for Form B of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form B).

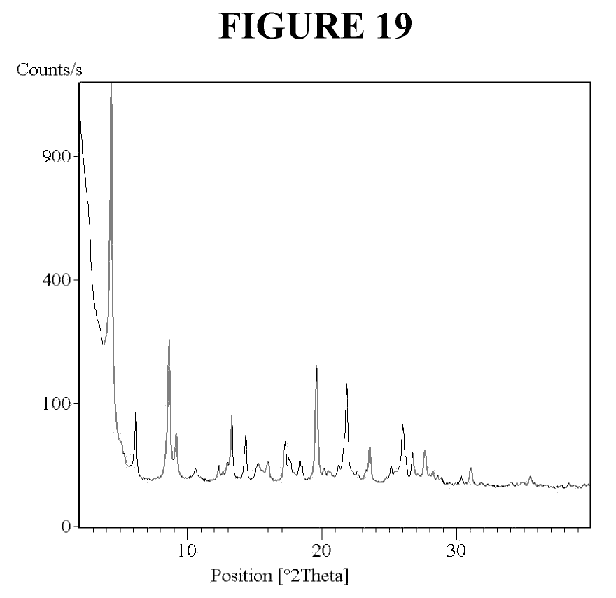

FIG. 19 shows the XRPD spectra for Form C of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form C).

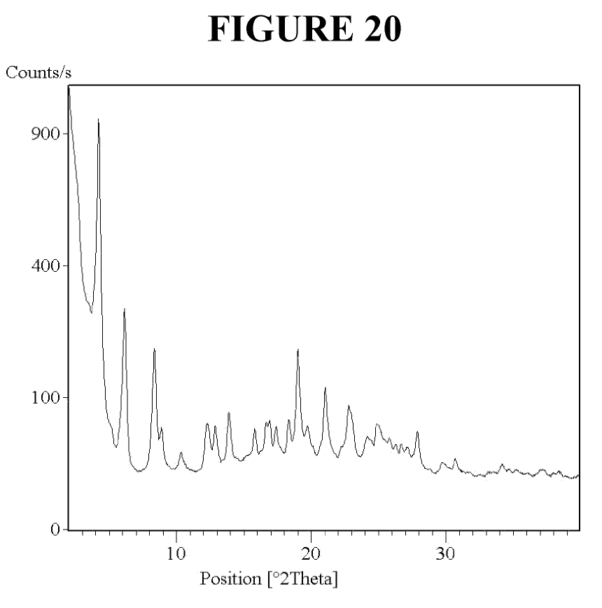

FIG. 20 shows the XRPD spectra for Form D of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form D).

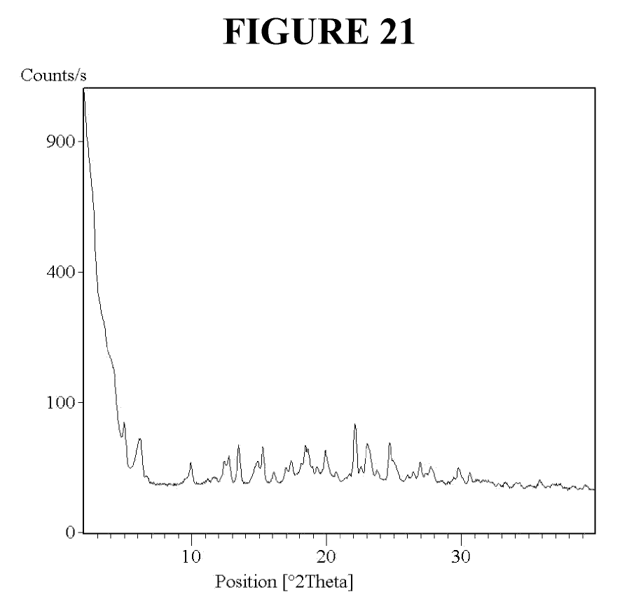

FIG. 21 shows the XRPD spectra for Form E of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form E).

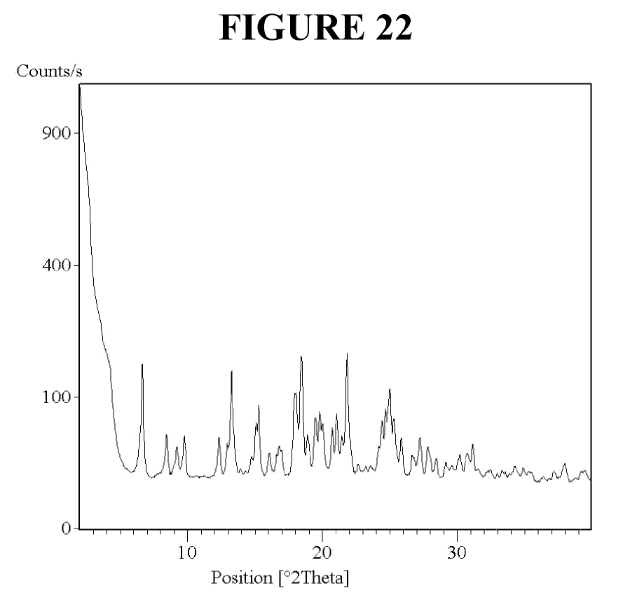

FIG. 22 shows the XRPD spectra for Form F of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Preparation 10, Form F).

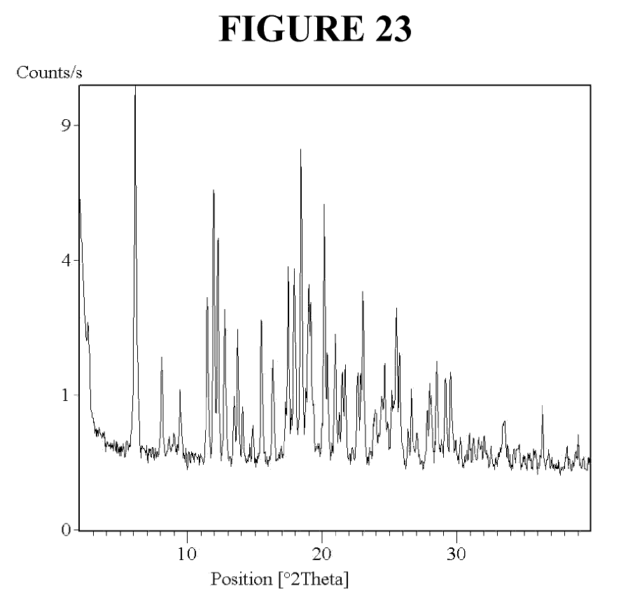

FIG. 23 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide bis(4-methylbenzenesulfonic acid) salt (Preparation 11, Form A).

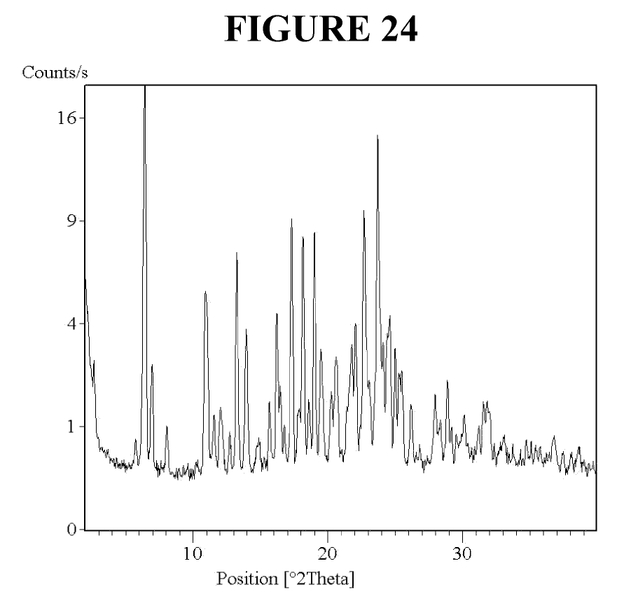

FIG. 24 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimethanesulfonic acid salt (Preparation 12, Form A).

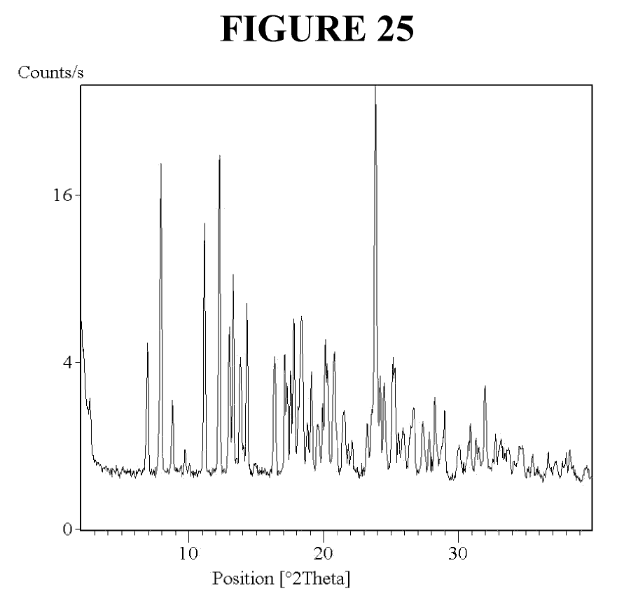

FIG. 25 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimaleic acid salt (Preparation 13, Form A).

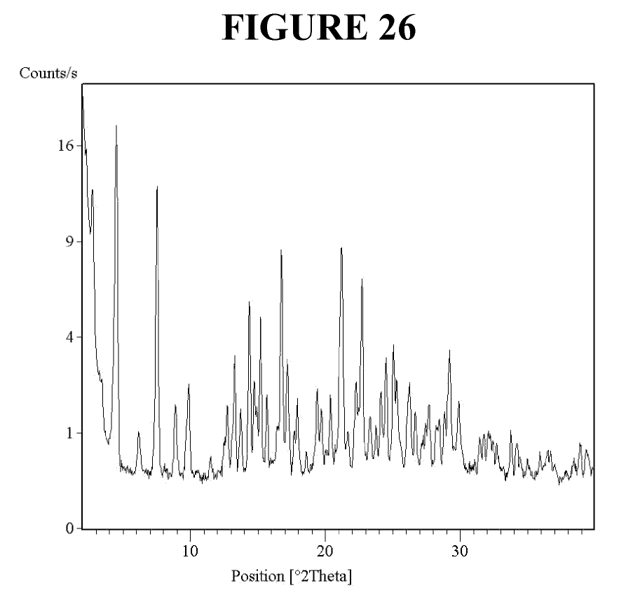

FIG. 26 shows the XRPD spectra for Form A of N-cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide disaccharin salt (Preparation 14, Form A).

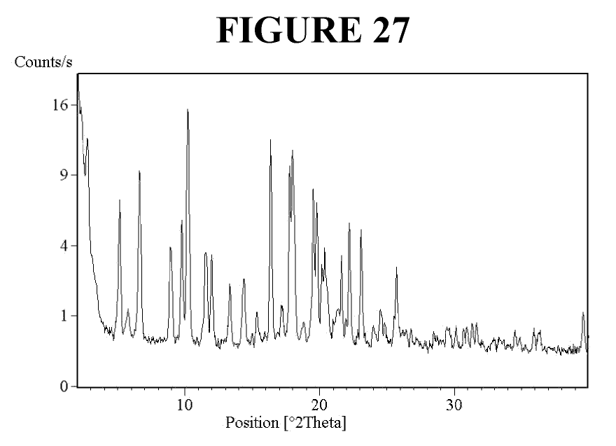

FIG. 27 shows the XRPD spectra for Form A of dicyclohexylammonium N-[(Benzyloxy)carbonyl]-N-[2-(3-fluorophenyl)ethyl]-β-alaninoate (Preparation 15, Form A).

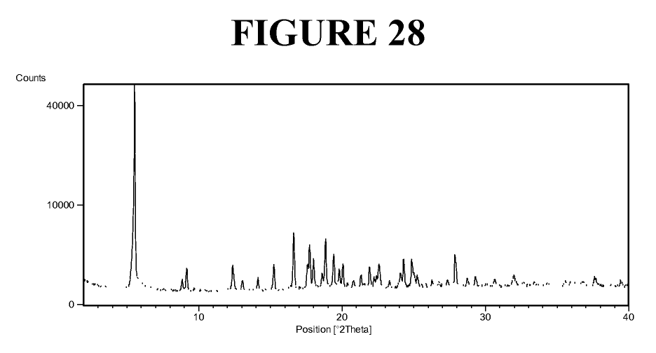

FIG. 28 shows the XRPD spectra for (R)-1-(4-Fluorophenethyl)-3-((S)-2-phenyl-2-(piperidin-1-yl)propanoyloxy)-1-azoniabicyclo[2.2.2]octane 4-methylbenzenesulphonate prepared according to Preparation 16.

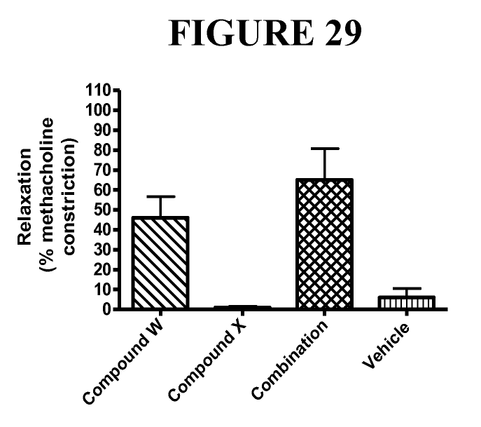

FIG. 29 shows the percentage relaxation to compound W (3 nM), compound X (1 nM), and the combination of compound W (3 nM) and compound X (1 nM) in guinea pig trachea in vitro in Example 8.

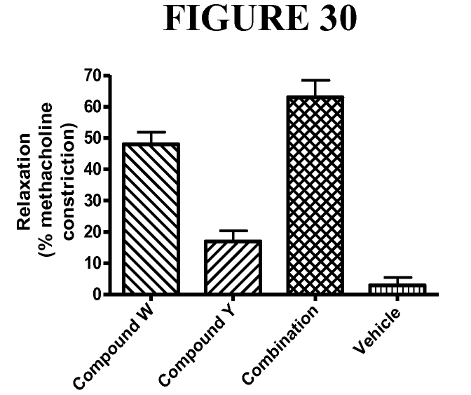

FIG. 30 shows the percentage relaxation to compound W (1 nM), compound Y (1 nM), and the combination of compound W (1 nM) and compound Y (1 nM) in guinea pig trachea in vitro in Example 8.

The compounds that are the subject of the above disclaimer are described in WO 2008/059245 and PCT/SE2009/050525.

The pharmaceutical product of the present invention comprises a first active ingredient and a second active ingredient, and it may comprise a third active ingredient. The third active ingredient can be chosen from the list of second active ingredients but would normally have a different mechanism of action. So, for example, the second active ingredient might be a muscarinic antagonist and the third active ingredient might be: a non-steroidal glucocorticosteroid receptor agonist, corticosteroid, a CCR1 antagonist or a PDE4 inhibitor.

In one particular aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof, and a second active ingredient selected from:

a non-steroidal Glucocorticoid Receptor (GR Receptor) Agonist;

an antioxidant;

a CCR1 antagonist;

a chemokine antagonist (not CCR1);

a corticosteroid;

a CRTh2 antagonist;

a DP1 antagonist;

an Histone Deacetylase Inducer;

an IKK2 inhibitor;

a COX inhibitor;

a lipoxygenase inhibitor;

a leukotriene receptor antagonist;

an MPO inhibitor;

a muscarinic antagonist;

a p38 inhibitor;

a PDE inhibitor;

a PPARγ agonist;

a protease inhibitor;

a Statin;

a thromboxane antagonist;

a vasodilator; or,

an ENAC blocker (Epithelial Sodium-channel blocker).

The first active ingredient, which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof, may be in the form of a solvate (such as a hydrate).

In another aspect of the invention a suitable salt of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide is, for example, a hydrochloride, hydrobromide (such as dihydrobromide), trifluoroacetate, sulphate, sulfonate, phosphate, acetate, fumarate, maleate, tartrate, lactate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate, 2-naphthalenesulphonate, 2,5-dichlorobenzenesulphonate, D-mandelate, L-mandelate, cinnamate, benzoate, adipate, esylate, malonate, mesitylate (2-mesitylenesulphonate), napsylate (2-naphthalenesulfonate), camsylate (camphor-10-sulphonate), formate, glutamate, glutarate, glycolate, hippurate (2-(benzoylamino)acetate), orotate, xylate (p-xylene-2-sulphonate), pamoic (2,2′-dihydroxy-1,1′-dinaphthylmethane-3,3′-dicarboxylate), palmitate or furoate.

In yet another aspect of the invention a suitable salt of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide is, for example, a hydrochloride, hydrobromide (such as dihydrobromide), trifluoroacetate, sulphate, phosphate, acetate, fumarate, maleate, tartrate, lactate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate, 2-naphthalenesulphonate, 2,5-dichlorobenzenesulphonate, D-mandelate, L-mandelate, cinnamate, benzoate, adipate, esylate, malonate, mesitylate (2-mesitylenesulphonate), napsylate (2-naphthalenesulfonate), camsylate (camphor-10-sulphonate), formate, glutamate, glutarate, glycolate, hippurate (2-(benzoylamino)acetate), orotate, xylate (p-xylene-2-sulphonate), pamoic (2,2′-dihydroxy-1,1′-dinaphthylmethane-3,3′-dicarboxylate), palmitate or furoate.

In a further aspect the present invention provides a pharmaceutical product wherein the first active ingredient is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt.

The first and second active ingredients can be administered simultaneously (either in a single pharmaceutical preparation {that is, the active ingredients are in admixture} or via separate preparations), or sequentially or separately via separate pharmaceutical preparations.

A non-steroidal glucocorticoid receptor (GR) agonist is, for example, a compound disclosed in WO 2006/046916.

An antioxidant is, for example, Allopurinol, Erdosteine, Mannitol, N-acetyl cysteine choline ester, N-acetyl cysteine ethyl ester, N-Acetylcysteine, N-Acetylcysteine amide or Niacin.

A CCR1 antagonist is, for example, a compound disclosed in WO2001/062728 or WO2001/098273, or a pharmaceutically acceptable salt thereof (such as a hydrochloride, trifluoroacetate, sulphate, (hemi)fumarate, benzoate, furoate or succinate salt); BX471 ((2R)-1-[[2-[(aminocarbonyl)amino]-4-chlorophenoxy]acetyl]-4-[(4-fluorophenyl)methyl]-2-methylpiperazine monohydrochloride) or CCX634.

Also, a CCR1 antagonist is, for example, a compound disclosed in WO2001/062728 or WO2001/098273 [such as N-(2-{(2S)-3[{(3R)-1-[(4-chlorophenyl)methyl]-3-pyrrolidinyl}amino]-2-hydroxypropoxy}-4-fluorophenyl)acetamide, N-(2{(2S)-3[{(3S)-1-[(4-chlorophenyl)methyl]-3-pyrrolidinyl}amino]-2-hydroxypropoxy}-4-fluorophenyl)acetamide, N-(2-{(2S)-3-[1-{(4-chlorobenzoyl)-4-piperidinyl}amino]-2-hydroxypropoxy}-4-hydroxyphenyl)acetamide, (2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid, (2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid, (2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid, Methyl (2-{[(2S)-3-{[1-(4-chlorobenzyl)piperidin-4-yl]amino}-2-hydroxypropyl]oxy}-4-fluorophenyl)propanoate, N-[2-({2S}-3-[(1-[4-chlorobenzyl]-4-piperidinyl)amino]-2-hydroxypropoxy)-4-chlorophenyl acetamide, N-[2-({2S}-3-[(1-[4-chlorobenzyl]-4-piperidinyl)amino]-2-hydroxy-2-methylpropoxy)-4-hydroxyphenyl]acetamide, N-[2-({2S}-3-[(1-[4-chlorobenzyl]-4-piperidinyl)amino]-2-hydroxy-2-methylpropoxy)-4-fluorophenyl]acetamide, N-[5-chloro-[2-({2S}-3-[(1-[4-chlorobenzyl]-4-piperidinyl)amino]-2-hydroxy-2-methylpropoxy)-4-hydroxyphenyl]acetamide, N-[5-chloro-[2-({2S}-3-[(1-[4-chlorobenzyl]-4-piperidinyl)amino]-2-hydroxy-2-methylpropoxy)-4-hydroxyphenyl]propaneamide, (2-{[(2S)-3-{[1-(4-chlorobenzyl)piperidin-4-yl]amino}-2-hydroxy-2-methylpropyl]oxy}-4-fluorophenyl)methanesulfonic acid, N-5-chloro-(2-{(2S)-3-[1-{(4-chlorobenzyl)-4-piperidinyl}amino]-2-hydroxypropoxy}-4-hydroxyphenyl)-N′-cyclopropyl-urea, N-(2-{(2S)-3-[1-{(4-chlorobenzyl)-4-piperidinyl}amino]-2-hydroxypropoxy}-phenyl)-N′-ethyl-urea, (2S)-1-(2-ethylphenoxy)-3-[(1-[4-chlorobenzyl]-4-piperidinyl)amino]propan-2-ol, (2S)-1-[2-(-hydroxyethyl)phenoxy]-2-methyl-3-[(1-[4-chlorobenzyl]-4-piperidinyl)amino]propan-2-ol, 2-({2S}-3-[(1-[4-chlorobenzyl]-4-piperidinyl)amino]-2-hydroxy-2-methylpropoxy)benzaldehyde, 2-({2S}-3-[(1-[4-chlorobenzyl]-4-piperidinyl)amino]-2-hydroxypropoxy)-N-cyclopropylbenzamide, Methyl 2-({2S}-3-[(1-[4-chlorobenzyl]-4-piperidinyl)amino]-2-hydroxypropoxy)-4-fluorobenzoate, N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide, N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide, 2-{[(2S)-3-(5-chloro-1′H,3H-spiro[1-benzofuran-2,4′-piperidin]-1′-yl)-2-hydroxypropyl]oxy}-4-hydroxy-N-methylbenzamide, 2-{[(2S)-3-(5-chloro-1′H,3H-spiro[1-benzofuran-2,4′-piperidin]-1′-yl)-2-hydroxypropyl]oxy}-4-hydroxybenzoic acid, N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide; 2-{[(2S)-3-(5-chloro-1′H,3H-spiro[2-benzofuran-1,4′-piperidin]-1′-yl)-2-hydroxypropyl]oxy}-4-hydroxy-N-methylbenzamide, N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide, 2-{[(2S)-3-(5-fluoro-1′H,3H-spiro[1-benzofuran-2,4′-piperidin]-1′-yl)-2-hydroxypropyl]oxy}-4-hydroxy-N-methylbenzamide, N-[2-({(2S)-3-[(2R)-5-chloro-1′H,3H-spiro[1-benzo furan-2,3′-pyrrolidin]-1′-yl]-2-hydroxypropyl}oxy)-4-hydroxyphenyl]acetamide, N-(2-{[(2S)-3-(5-chloro-1′H,3H-spiro[1-benzofuran-2,4′-piperidin]-1′-yl)-2-hydroxypropyl]oxy}-4-hydroxyphenyl)urea, 4-fluoro-2-{[(2S)-3-(5-fluoro-1′H,3H-spiro[1-benzo furan-2,4′-piperidin]-1′-yl)-2-hydroxypropyl]oxy}benzoic acid, N-(2-{[(2S)-3-(5-chloro-1′H,3H-spiro[1-benzofuran-2,4′-piperidin]-1′-yl)-2-hydroxypropyl]oxy}-4-fluorophenyl)urea, N-(2-{[(2S)-2-amino-3-(5-fluoro-1′H,3H-spiro[1-benzofuran-2,4′-piperidin]-1′-yl)propyl]oxy}-4-hydroxyphenyl)acetamide, 2-[(2S)-3-(5-chloro spiro[benzofuran-2(3H), 4′-piperidin]-1′-yl)-2-hydroxypropoxy]-benzaldehyde, (αS)-5-chloro-α-[[2-(2-hydroxyethyl)phenoxy]methyl]-Spiro[benzofuran-2(3H), 4′-piperidine]-1′-ethanol, (αS)-5-chloro-a-[[2-(hydroxymethyl)phenoxy]methyl]-Spiro[benzofuran-2(3H), 4′-piperidine]-1′-ethanol, N-(2-{[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide, 2-Chloro-5-{[(2S)-3-(5-chloro-1′H,3H-spiro[1-benzofuran-2,4′-piperidin]-1′-yl)-2-hydroxypropyl]oxy}-(4-{acetylamino}phenoxy)acetic acid, 5-{[(2S)-3-(5-Chloro-1′H,3H-spiro[1-benzo furan-2,4′-piperidin]-1′-yl)-2-hydroxypropyl]oxy}-(4-{acetylamino}phenoxy)acetic acid, {2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid, 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, (2-Chloro-5-{[(2S)-3-(5-chloro-1′H,3H-spiro[1-benzo furan-2,4′-piperidin]-1′-yl)-2-hydroxypropyl]oxy}-4-{[(3S)-3-hydroxypyrrolidin-1-yl]carbonyl}phenoxy)acetic acid, 5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid, 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic, 5-Chloro-2-{[(2S)-3-(5-chloro-1′H,3H-spiro[1-benzo furan-2,4′-piperidin]-1′-yl)-2-hydroxypropyl]oxy}-4-(3,3,3-trifluoropropoxy)benzoic acid, N-(2-{3-[5-chloro-1′H,3H-spiro[1-benzo furan-2,4′-piperidin]-1′-yl]propoxy}phenyl)acetamide, Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid, N-(2-{[(2S)-3-({spiro[indole-2-4′-piperidin]-3(1H)-one}-1′-yl)-2-hydroxypropyl]oxy}-4-hydroxyphenyl)acetamide, or (2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid, or a pharmaceutically acceptable salt thereof (for example as described above; (such as a hydrochloride, trifluoroacetate, sulphate, (hemi)fumarate, benzoate, furoate or succinate salt))]; BX471 ((2R)-1-[[2-[(aminocarbonyl)amino]-4-chlorophenoxy]acetyl]-4-[(4-fluorophenyl)methyl]-2-methylpiperazine monohydrochloride); or CCX634.

Also, a CCR1 antagonist is, for example, N-{2-R(2S)-3-{[1-(4-chlorobenzyl)piperidin-4-yl]amino}-2-hydroxy-2-methylpropyl)oxy]-4-hydroxyphenyl}acetamide (see WO 2003/051839), or, 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid (see PCT publication no. WO 2008/010765), or a pharmaceutically acceptable salt thereof (for example a hydrochloride, sulphate, (hemi)fumarate, benzoate, furoate or succinate salt).

A chemokine antagonist (other than a CCR1 antagonist), for example, 656933 (N-(2-bromophenyl)-N′-(4-cyano-1H-1,2,3-benzotriazol-7-yl)urea), 766994 (4-({[({[(2R)-4-(3,4-dichlorobenzyl)morpholin-2-yl]methyl}amino)carbonyl]-amino}methyl)benzamide), CCX-282, CCX-915, Cyanovirin N, E-921, NCB-003284, INCB-9471, Maraviroc, MLN-3701, MLN-3897, T-487 (N-{1-[3-(4-ethoxyphenyl)-4-oxo-3,4-dihydropyrido[2,3-d]pyrimidin-2-yl]ethyl}-N-(pyridin-3-ylmethyl)-2-[4-(trifluoromethoxy)phenyl]acetamide) or Vicriviroc.

A corticosteroid is, for example, Alclometasone dipropionate, Amelometasone, Beclomethasone dipropionate, Budesonide, Butixocort propionate, Ciclesonide, Clobetasol propionate, Desisobutyrylciclesonide, Etiprednol dicloacetate, Fluocinolone acetonide, Fluticasone Furoate, Fluticasone propionate, Loteprednol etabonate (topical) or Mometasone furoate.

A CRTh2 antagonist is, for example, a compound from WO 2004/106302 or WO 2005/018529.

A DP1 antagonist is, for example, L888839 or MK0525.

An histone deacetylase inducer is, for example, ADC4022, Aminophylline, a Methylxanthine or Theophylline.

An IKK2 inhibitor is, for example, 2-{[2-(2-Methylamino-pyrimidin-4-yl)-1H-indole-5-carbonyl]-amino}-3-(phenyl-pyridin-2-yl-amino)-propionic acid.

A COX inhibitor is, for example, Celecoxib, Diclofenac sodium, Etodolac, Ibuprofen, Indomethacin, Meloxicam, Nimesulide, OC1768, OC2125, OC2184, OC499, OCD9101, Parecoxib sodium, Piceatannol, Piroxicam, Rofecoxib or Valdecoxib.

A lipoxygenase inhibitor is, for example, Ajulemic acid, Darbufelone, Darbufelone mesilate, Dexibuprofen lysine (monohydrate), Etalocib sodium, Licofelone, Linazolast, Lonapalene, Masoprocol, MN-001, Tepoxalin, UCB-35440, Veliflapon, ZD-2138, ZD-4007 or Zileuton ((±)-1-(1-Benzo[b]thien-2-ylethyl)-1-hydroxyurea).

A leukotriene receptor antagonist is, for example, Ablukast, Iralukast (CGP 45715A), Montelukast, Montelukast sodium, Ontazolast, Pranlukast, Pranlukast hydrate (mono Na salt), Verlukast (MK-679) or Zafirlukast.

An MPO Inhibitor is, for example, a Hydroxamic acid derivative (N-(4-chloro-2-methyl-phenyl)-4-phenyl-4-[[(4-propan-2-ylphenyl)sulfonylamino]methyl]piperidine-1-carboxamide), Piceatannol or Resveratrol.

A muscarinic antagonist is, for example, Aclidinium bromide, Glycopyrrolate (such as R,R-, R-,S-, S,R-, or S,S-glycopyrronium bromide), Oxitropium bromide, Pirenzepine, telenzepine, Tiotropium bromide, 3(R)-(2-hydroxy-2,2-dithien-2-ylacetoxy)-1-(3-phenoxypropyl)-1-azoniabicyclo[2.2.2]octane bromide (see WO 01/04118), 3(R)-1-phenethyl-3-(9H-xanthene-9-carbonyloxy)-1-azoniabicyclo[2.2.2]octane bromide or (3R)-3-[(2S)-2-cyclopentyl-2-hydroxy-2-thien-2-ylacetoxy]-1-(2-phenoxyethyl)-1-azoniabicyclo[2.2.2]actane bromide (see WO 01/04118); or a quaternary ammonium salt (such as [2-((S)-Cyclohexyl-hydroxy-phenyl-methyl)-oxazol-5-ylmethyl]-dimethyl-(3-phenoxy-propyl)-ammonium salt, [2-((R)-Cyclohexyl-hydroxy-phenyl-methyl)-oxazol-5-ylmethyl]-dimethyl-(3-phenoxy-propyl)-ammonium salt, [2((R)-Cyclohexyl-hydroxy-phenyl-methyl)-oxazol-5-ylmethyl]-dimethyl-(2-phenethyloxy-ethyl)-ammonium salt, [2-(4(R)-Cyclohexyl-hydroxy-phenyl-methyl)-oxazol-5-ylmethyl]-[3-(3,4-dichloro-phenoxy)-propyl]dimethyl-ammonium salt, [2-((R)-Cyclohexyl-hydroxy-phenyl-methyl)-oxazol-5-ylmethyl]-[2-(3,4-dichloro-benzyloxy)-ethyl]-dimethyl-ammonium salt, [2-(4-Chloro-benzyloxy)-ethyl]-[2-((R)-Cyclohexyl-hydroxy-phenyl-methyl)-oxazol-5-ylmethyl]-dimethyl-ammonium salt, or (R)-1-[2-(4-Fluoro-phenyl)-ethyl]-3-((S)-2-phenyl-2-piperidin-1-yl-propionyloxy)-1-azonia-bicyclo[2.2.2]octane; wherein the counter-ion is, for example, chloride, bromide, sulfate, methanesulfonate, benzenesulfonate (besylate), toluenesulfonate (tosylate), napthalene-bissulfonate (napadisylate), phosphate, acetate, citrate, lactate, tartrate, mesylate, maleate, fumarate or succinate).

In one aspect of the invention a muscarinic antagonist is Aclidinium bromide, Glycopyrrolate (such as R,R-, R,S-, S,R-, or S,S-glycopyrronium bromide), Oxitropium bromide, Pirenzepine, telenzepine or Tiotropium bromide.

In another aspect of the invention a muscarinic antagonist is Glycopyrrolate (such as R,R-, R-,S-, S,R-, or S,S-glycopyrronium bromide) or Tiotropium bromide.

In yet another aspect a muscarinic antagonist is (R)-1-[2-(4-Fluoro-phenyl)-ethyl]-3-((S)-2-phenyl-2-piperidin-1-yl-propionyloxy)-1-azonia-bicyclo[2.2.2]octane (see WO2008/075005); wherein the counter-ion is, for example, chloride, bromide, sulfate, methanesulfonate, benzenesulfonate (besylate), toluenesulfonate (tosylate), napthalene-bissulfonate (napadisylate), phosphate, acetate, citrate, lactate, tartrate, mesylate, maleate, fumarate or succinate).

A p38 Inhibitor is, for example, a compound from WO 2005/042502, 681323, 856553, AMG548 (2-[[(2S)-2-amino-3-phenylpropyl]amino]-3-methyl-5-(2-naphthalenyl)-6-(4-pyridinyl)-4(3H)-pyrimidinone), Array-797, AZD6703, Doramapimod, KC-706, PH 797804, R1503, SC-80036, SCIO469, 6-chloro-5-[[(2S,5R)-4-[(4-fluorophenyl)methyl]-2,5-domethyl-1-piperazinyl]carbonyl]-N,N,1-trimethyl-α-oxo-1H-indole-3-acetamide, VX702 or VX745 (5-(2,6-dichlorophenyl)-2-(phenylthio)-6H-pyrimido[1,6-b]pyridazin-6-one).

A PDE Inhibitor: such as a PDE4 inhibitor is, for example, 256066, Arofylline (3-(4-chlorophenyl)-3,7-dihydro-1-propyl-1H-Purine-2,6-dione), AWD 12-281 (N-(3,5-dichloro-4-pyridinyl)-1-[(4-fluorophenyl)methyl]-5-hydroxy-α-oxo-1H-indole-3-acetamide), BAY19-8004 (Bayer), CDC-801 (Calgene), Celgene compound 4(3R)-(3-(3,4-dimethoxyphenyl)-1,3-dihydro-1-oxo-2H-isoindole-2-propanamide), Cilomilast (cis-4-cyano-4-[3-(cyclopentyloxy)-4-methoxyphenyl]-cyclohexanecarboxylic acid), a compound in WO2006098353 from Kyowa Hakko Kogyo Co. Ltd. Japan, 2-(3,5-dichloro-4-pyridinyl)-1-(7-methoxyspiro[1,3-benzodioxole-2,1′-cyclopentan]-4-yl)ethanone (CAS number 185406-34-2)), Compound from Pfizer (2-(3,4-difluorophenoxy)-5-fluoro-N-[cis-4-[(2-hydroxy-5-methylbenzoyl)amino]cyclohexyl]-)-3-pyridinecarboxamide), Compound from Pfizer (2-(3,4-difluorophenoxy)-5-fluoro-N-[cis-4-[[2-hydroxy-5-(hydroxymethyl)benzoyl]amino]cyclohexyl]-3-pyridinecarboxamide,), CT2820, GPD-1116, Ibudilast, IC 485, KF 31334, KW-4490 (Kyowa Hakko Kogyo), Lirimilast ([2-(2,4-dichlorobenzoyl)-6-[(methylsulfonyl)oxy]-3-benzofuranyl p-urea), Merck Compound (N-cyclopropyl-1,4-dihydro-4-oxo-1-[3-(3-pyridinylethynyl)phenyl-)-1,8-naphthyridine-3-carboxamide), Oglemilast (N-(3,5-dichloro-4-pyridinyl)-4-(difluoromethoxy)-8-[(methylsulfonyl)amino])-1-dibenzofurancarboxamide), ONO6126, ORG 20241 (4-(3,4-dimethoxyphenyl)-N-hydroxy-)-2-thiazolecarboximidamide), PD189659/PD168787 (Parke-Davis), Pentoxifylline (3,7-dihydro-3,7-dimethyl-1-(5-oxohexyl-)-1H-purine-2,6-dione), Pfizer compound (5-fluoro-N-[4-[(2-hydroxy-4-methyl-benzoyl)amino]cyclohexyl]-2-(thian-4-yloxy)pyridine-3-carboxamide), Pfizer UK 500,001, Piclamilast (3-(cyclopentyloxy)-N-(3,5-dichloro-4-pyridinyl)-4-methoxy-benzamide), PLX-369 (WO 2006026754), Roflumilast (3-(cyclopropylmethoxy)-N-(3,5-dichloro-4-pyridinyl)-4-(difluoromethoxy)benzamide), SCH 351591 (N-(3,5-dichloro-1-oxido-4-pyridinyl)-8-methoxy-2-(trifluoromethyl)-5-quinolinecarboxamide), SelCID™ CC-10004 (Calgene), T-440 (Tanabe), Tetomilast (6-[2-(3,4-diethoxyphenyl)-4-thiazolyl]-2-pyridinecarboxylic acid), Tofimilast (9-cyclopentyl-7-ethyl-6,9-dihydro-3-(2-thienyl)-5H-pyrazolo[3,4-c]-1,2,4-triazolo[4,3-a]pyridine), TPI 1100, UCB 101333-3 (N,2-dicyclopropyl-6-(hexahydro-1H-azepin-1-yl)-5-methyl-4-pyrimidinamine), V-11294A (Napp), VM554/VM565 (Vernalis), or Zardaverine (6-[4-(difluoromethoxy)-3-methoxyphenyl]-3(2H)-pyridazinone).

A PDE5 Inhibitor is, for example, Gamma-glutamyl[s-(2-iodobenzyl)cysteinyl]glycine, Tadalafil, Vardenafil, sildenafil, 4-phenyl-methylamino-6-chloro-2-(1-imidazolyl)-quinazoline, 4-phenyl-methylamino-6-chloro-2-(3-pyridyl)-quinazoline, 1,3-dimethyl-6-(2-propoxy-5-methanesulphonylamidophenyl)-1,5-dihydropyrazolo[3,4-d]pyrimidin-4-one or 1-cyclopentyl-3-ethyl-6-(3-ethoxy-4-pyridyl)-pyrazolo[3,4-d]pyrimidin-4-one.

A PPARγ agonist is, for example, Pioglitazone, Pioglitazone hydrochloride, Rosiglitazone Maleate, Rosiglitazone Maleate ((−)-enantiomer, free base), Rosiglitazone maleate/Metformin hydrochloride or Tesaglitizar.

A Protease Inhibitor is, for example, Alpha1-antitrypsin proteinase Inhibitor, EPI-HNE4, UT-77, ZD-0892 or a compound from WO 2006/004532, WO 2005/026123, WO 2002/0744767 or WO 22002/074751; or a TACE Inhibitor (for example DPC-333, Sch-709156 or Doxycycline).

A Statin is, for example, Atorvastatin, Lovastatin, Pravastatin, Rosuvastatin or Simvastatin.

A Thromboxane Antagonist is, for example, Ramatroban or Seratrodast.

A Vasodilator is, for example, A-306552, Ambrisentan, Avosentan, BMS-248360, BMS-346567, BMS-465149, BMS-509701, Bosentan, BSF-302146 (Ambrisentan), Calcitonin Gene-related Peptide, Daglutril, Darusentan, Fandosentan potassium, Fasudil, Iloprost, KC-12615 (Daglutril), KC-12792 2AB (Daglutril), Liposomal treprostinil, PS-433540, Sitaxsentan sodium, Sodium Ferulate, TBC-11241 (Sitaxsentan), TBC-3214 (N-(2-acetyl-4,6-dimethylphenyl)-3-[[(4-chloro-3-methyl-5-isoxazolyl)amino]sulfonyl]-2-thiophenecarboxamide), TBC-3711, Trapidil, Treprostinil diethanolamine or Treprostinil sodium.

An ENAC (Epithelial Sodium-channel blocker) is, for example, Amiloride, Benzamil, Triamterene, 552-02, PSA14984, PSA25569, PSA23682 or AER002.

All the above second et seq active ingredients may be in the form of solvates, for example hydrates.

In one particular aspect the present invention provides a pharmaceutical product comprising the first and second active ingredients in admixture. Alternatively, the pharmaceutical product may, for example, be a kit comprising a preparation of the first active ingredient and a preparation of the second active ingredient and, optionally, instructions for the simultaneous, sequential or separate administration of the preparations to a patient in need thereof.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient selected from:

a non-steroidal Glucocorticoid Receptor (GR Receptor) Agonist;

a CCR1 antagonist;

a chemokine antagonist (not CCR1);

a corticosteroid;

an IKK2 inhibitor;

a muscarinic antagonist;

a p38 inhibitor; or,

a PDE inhibitor;

provided that the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or,
(R)-1-[(2-Methyl-pyridin-4-ylcarbamoyl)-methyl]-3-(1-phenyl-cycloheptanecarbonyloxy)-1-azonia-bicyclo[2.2.2]octane X;

wherein X represents a pharmaceutically acceptable anion of a mono or polyvalent acid.

In yet another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient selected from:

a non-steroidal Glucocorticoid Receptor (GR Receptor) Agonist;

a CCR1 antagonist;

a chemokine antagonist (not CCR1);

a corticosteroid;

an IKK2 inhibitor;

a muscarinic antagonist;

a p38 inhibitor; or,

a PDE inhibitor.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient which is a non-steroidal Glucocorticoid Receptor (GR) Agonist for example, a compound disclosed in WO 2006/046916.

In yet another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient which is a CCR1 antagonist, for example, a compound disclosed in WO2001/062728 or WO2001/098273 [such as N-(2{(2S)-3[{(3R)-1-[(4-chlorophenyl)methyl]-3-pyrrolidinyl}amino]-2-hydroxypropoxy}-4-fluorophenyl)acetamide, N-(2{(2S)-3[{(3S)-1-[(4-chlorophenyl)methyl]-3-pyrrolidinyl}amino]-2-hydroxypropoxy}-4-fluorophenyl)acetamide, N-(2-{(2S)-3-[1-{(4-chlorobenzoyl)-4-piperidinyl}amino]-2-hydroxypropoxy}-4-hydroxyphenyl)acetamide, (2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid, (2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid, (2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid, (2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid, Methyl (2-{[(2S)-3-{[1-(4-chlorobenzyl)piperidin-4-yl]amino}-2-hydroxypropyl]oxy}-4-fluorophenyl)propanoate, N-[2-({2S}-3-[(1-[4-chlorobenzyl]-4-piperidinyl)amino]-2-hydroxypropoxy)-4-chlorophenyl acetamide, N-[2-({2S}-3-[(1-[4-chlorobenzyl]-4-piperidinyl)amino]-2-hydroxy-2-methylpropoxy)-4-hydroxyphenyl]acetamide, N-[2-({2S}-3-[(1-[4-chlorobenzyl]-4-piperidinyl)amino]-2-hydroxy-2-methylpropoxy)-4-fluorophenyl]acetamide, N-[5-chloro-[2-({2S}-3-[(1-[4-chlorobenzyl]-4-piperidinyl)amino]-2-hydroxy-2-methylpropoxy)-4-hydroxyphenyl]acetamide, N-[5-chloro-[2-({2S}-3-[(1-[4-chlorobenzyl]-4-piperidinyl)amino]-2-hydroxy-2-methylpropoxy)-4-hydroxyphenyl]propaneamide, (2-{[(2S)-3-{[1-(4-chlorobenzyl)piperidin-4-yl]amino}-2-hydroxy-2-methylpropyl]oxy}-4-fluorophenyl)methanesulfonic acid, N-5-chloro-(2-{(2S)-3-[1-{(4-chlorobenzyl)-4-piperidinyl}amino]-2-hydroxypropoxy}-4-hydroxyphenyl)-N′-cyclopropyl-urea, N-(2-{(2S)-3-[1-{(4-chlorobenzyl)-4-piperidinyl}amino]-2-hydroxypropoxy}-phenyl)-N′-ethyl-urea, (2S)-1-(2-ethylphenoxy)-3[(1-[4-chlorobenzyl]-4-piperidinyl)amino]propan-2-ol, (2S)-1-[2-(-hydroxyethyl)phenoxy]-2-methyl-3[(1-[4-chlorobenzyl]-4-piperidinyl)amino]propan-2-ol, 2-({2S}-3-[(1-[4-chlorobenzyl]-4-piperidinyl)amino]-2-hydroxy-2-methylpropoxy)benzaldehyde, 2-({2S}-3-[(1-[4-chlorobenzyl]-4-piperidinyl)amino]-2-hydroxypropoxy)-N-cyclopropylbenzamide, Methyl 2-({2S}-3-[(1-[4-chlorobenzyl]-4-piperidinyl)amino]-2-hydroxypropoxy)-4-fluorobenzoate, N-(2-{[(2S)-3-(5-chloro-1′H,3H-spiro[1-benzofuran-2,4′-piperidin]-1′-yl)-2-hydroxypropyl]oxy}-4-hydroxyphenyl)acetamide, N-(2-{[(2S)-3-(5-chloro-1′H-spiro[1,3-benzodioxole-2,4′-piperidin]-1′-yl)-2-hydroxypropyl]oxy}-4-hydroxyphenyl)acetamide, 2-{[(2S)-3-(5-chloro-1′H,3H-spiro[1-benzofuran-2,4′-piperidin]-1′-yl)-2-hydroxypropyl]oxy}-4-hydroxy-N-methylbenzamide, 2-{[(2S)-3-(5-chloro-1′H,3H-spiro[1-benzofuran-2,4′-piperidin]-1′-yl)-2-hydroxypropyl]oxy}-4-hydroxybenzoic acid, N-(2-{[(2S)-3-(5-chloro-1′H,3H-spiro[2-benzofuran-1,4′-piperidin]-1′-yl)-2-hydroxypropyl]oxy}-4-hydroxyphenyl)acetamide; 2-{[(2S)-3-(5-chloro-1′H,3H-spiro[2-benzofuran-1,4′-piperidin]-1′-yl)-2-hydroxypropyl]oxy}-4-hydroxy-N-methylbenzamide, N-(2-{[(2S)-3-(5-fluoro-1′H,3H-spiro[1-benzofuran-2,4′-piperidin]-1′-yl)-2-hydroxypropyl]oxy}-4-hydroxyphenyl)acetamide, 2-{[(2S)-3-(5-fluoro-1′H,3H-spiro[1-benzofuran-2,4′-piperidin]-1′-yl)-2-hydroxypropyl]oxy}-4-hydroxy-N-methylbenzamide, N-[2-({(2S)-3-[(2R)-5-chloro-1′H,3H-spiro[1-benzo furan-2,3′-pyrrolidin]-1′-yl]-2-hydroxypropyl 1 oxy)-4-hydroxyphenyl]acetamide, N-(2-{[(2S)-3-(5-chloro-1′H,3H-spiro[1-benzofuran-2,4′-piperidin]-1′-yl)-2-hydroxypropyl]oxy}-4-hydroxyphenyl)urea, 4-fluoro-2-{[(2S)-3-(5-fluoro-1′H,3H-spiro[1-benzofuran-2,4′-piperidin]-1′-yl)-2-hydroxypropyl]oxy}benzoic acid, N-(2-{[(2S)-3-(5-chloro-1′H,3H-spiro[1-benzofuran-2,4′-piperidin]-1′-yl)-2-hydroxypropyl]oxy}-4-fluorophenyl)urea, N-(2-{[(2S)-2-amino-3-(5-fluoro-1′H,3H-spiro[1-benzofuran-2,4′-piperidin]-1′-yl)propyl]oxy}-4-hydroxyphenyl)acetamide, 2-[(2S)-3-(5-chlorospiro[benzofuran-2(3H), 4′-piperidin]-1′-yl)-2-hydroxypropoxy]-benzaldehyde, (αS)-5-chloro-a-[[2-(2-hydroxyethyl)phenoxy]methyl]-Spiro[benzofuran-2(3H), 4′-piperidine]-1′-ethanol, (αS)-5-chloro-a-[[2-(hydroxymethyl)phenoxy]methyl]-Spiro[benzofuran-2(3H), 4′-piperidine]-1′-ethanol, N-(2-{[(2S)-3-(5-chloro-1′H,3H-spiro[1-benzofuran-2,4′-piperidin]-1′-yl)-2-hydroxypropyl]oxy}-5-chloro-4-hydroxyphenyl)acetamide, 2-Chloro-5-{[(2S)-3-(5-chloro-1′H,3H-spiro[1-benzofuran-2,4′-piperidin]-1′-yl)-2-hydroxypropyl]oxy}-(4-{acetylamino}phenoxy)acetic acid, 5-{[(2S)-3-(5-Chloro-1′H,3H-spiro[1-benzofuran-2,4′-piperidin]-1′-yl)-2-hydroxypropyl]oxy}-(4-{acetylamino}phenoxy)acetic acid, {2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}acetic acid, 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, (2-Chloro-5-{[(2S)-3-(5-chloro-1′H,3H-spiro[1-benzofuran-2,4′-piperidin]-1′-yl)-2-hydroxypropyl]oxy}-4-{[(3S)-3-hydroxypyrrolidin-1-yl]carbonyl}phenoxy)acetic acid, 5-Chloro-2-{[(2S)-3-(5-chloro-1′H,3H-spiro[1-benzofuran-2,4′-piperidin]-1′-yl)-2-hydroxypropyl]oxy}-4-(cyanomethoxy)benzoic acid, 2-{[(2S)-3-(5-chloro-1′H,3H-spiro[1-benzofuran-2,4′-piperidin]-1′-yl)-2-hydroxypropyl]oxy}-5-chloro-4-(2,2-difluoroethoxy)benzoic, 5-Chloro-2-{[(2S)-3-(5-chloro-1′H,3H-spiro[1-benzofuran-2,4′-piperidin]-1′-yl)-2-hydroxypropyl]oxy}-4-(3,3,3-trifluoropropoxy)benzoic acid, N-(2-{3-[5-chloro-1′H,3H-spiro[1-benzofuran-2,4′-piperidin]-1′-yl]propoxy}phenyl)acetamide, Methyl 3-(2-{[(2S)-3-(5-chloro-1′H3H-spiro[1-benzofuran-2,4′-piperidin]-1′-yl)-2-hydroxypropyl]oxy}-4-fluorophenyl)propanoic acid, N-(2-{[(2S)-3-({spiro[indole-2-4′-piperidin]-3(1H)-one}-1′-yl)-2-hydroxypropyl]oxy}-4-hydroxyphenyl)acetamide, or (2-{[(2S)-3-(5-Chloro-1′H,3H-spiro[1-benzofuran-2,4′-piperidin]-1′-yl)-2-hydroxypropyl]oxy}-4-fluorophenyl)methanesulfonic acid, or a pharmaceutically acceptable salt thereof (for example as described above; (such as a hydrochloride, trifluoroacetate, sulphate, (hemi)fumarate, benzoate, furoate or succinate salt))]; BX471 ((2R)-1-[[2-[(aminocarbonyl)amino]-4-chlorophenoxy]acetyl]-4-[(4-fluorophenyl)methyl]-2-methylpiperazine monohydrochloride); or CCX634.

In another aspect a CCR1 antagonist is N-{2-R(2S)-3-{[1-(4-chlorobenzyl)piperidin-4-yl]amino}-2-hydroxy-2-methylpropyl)oxy]-4-hydroxyphenyl}acetamide, or, 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid, or a pharmaceutically acceptable salt thereof (for example a hydrochloride, sulphate, (hemi)fumarate, benzoate, furoate or succinate salt). For example N-{2-R(2S)-3-{[1-(4-chlorobenzyl)piperidin-4-yl]amino}-2-hydroxy-2-methylpropyl)oxy]-4-hydroxyphenyl}acetamide as a benzoate salt, or, 2-{2-Chloro-5-{[(2S)-3-(5-chloro-1′H,3H-spiro[1-benzofuran-2,4′-piperidin]-1′-yl)-2-hydroxypropyl]oxy}-4-[(methylamino)carbonyl]phenoxy}-2-methylpropanoic acid as the free acid.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient which is a chemokine antagonist (not CCR1), for example, 656933 (N-(2-bromophenyl)-N′-(4-cyano-1H-1,2,3-benzotriazol-7-yl)urea), 766994 (4-({[({[(2R)-4-(3,4-dichlorobenzyl)morpholin-2-yl]methyl}amino)carbonyl]-amino}methyl)benzamide), CCX-282, CCX-915, Cyanovirin N, E-921, NCB-003284, NCB-9471, Maraviroc, MLN-3701, MLN-3897, T-487 (N-{1-[3-(4-ethoxyphenyl)-4-oxo-3,4-dihydropyrido[2,3-d]pyrimidin-2-yl]ethyl}-N-(pyridin-3-ylmethyl)-2-[4-(trifluoromethoxy)phenyl]acetamide) or Vicriviroc.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a corticosteroid, for example, Alclometasone dipropionate, Amelometasone, Beclomethasone dipropionate, Budesonide, Butixocort propionate, Ciclesonide, Clobetasol propionate, Desisobutyrylciclesonide, Etiprednol dicloacetate, Fluocinolone acetonide, Fluticasone Furoate, Fluticasone propionate, Loteprednol etabonate (topical) or Mometasone furoate.

In one embodiment of the present invention the corticosteroid is selected from budesonide, fluticasone propionate, fluticasone fruoate mometasone furoate, beclomethasone dipropionate or butixocort propionate ester.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a corticosteroid, for example, Budesonide, Fluticasone Furoate or Fluticasone propionate.

In one embodiment of the present invention the corticosteroid is budesonide. Budesonide and its preparation is described, for example, in Arzneimittel-Forschung (1979), 29 (11), 1687-1690, DE 2,323,215 and U.S. Pat. No. 3,929,768. Presently available formulations of budesonide are marketed under the tradename ‘Entocort®’.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is an IKK2 inhibitor, for example, 2-{[2-(2-Methylamino-pyrimidin-4-yl)-1H-indole-5-carbonyl]-amino}-3-(phenyl-pyridin-2-yl-amino)-propionic acid.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a muscarinic antagonist, for example, Aclidinium bromide, Glycopyrrolate (such as R,R-, R,S-, S,R-, or S,S-glycopyrronium bromide), Oxitropium bromide, Pirenzepine, telenzepine, Tiotropium bromide, 3(R)-(2-hydroxy-2,2-dithien-2-ylacetoxy)-1-(3-phenoxypropyl)-1-azoniabicyclo[2.2.2]octane bromide (see WO 01/04118), or 3(R)-1-phenethyl-3-(9H-xanthene-9-carbonyloxy)-1-azoniabicyclo[2.2.2]octane bromide or (3R)-3-[(2S)-2-cyclopentyl-2-hydroxy-2-thien-2-ylacetoxy]-1-(2-phenoxyethyl)-1-azoniabicyclo[2.2.2]actane bromide (see WO 01/04118); or a quaternary ammonium salt (such as [2-((S)-Cyclohexyl-hydroxy-phenyl-methyl)-oxazol-5-ylmethyl]-dimethyl-(3-phenoxy-propyl)-ammonium salt, [2-((R)-Cyclohexyl-hydroxy-phenyl-methyl)-oxazol-5-ylmethyl]-dimethyl-(3-phenoxy-propyl)-ammonium salt, [2-((R)-Cyclohexyl-hydroxy-phenyl-methyl)-oxazol-5-ylmethyl]-dimethyl-(2-phenethyloxy-ethyl)-ammonium salt, [2-((R)-Cyclohexyl-hydroxy-phenyl-methyl)-oxazol-5-ylmethyl]-[3-(3,4-dichloro-phenoxy)-propyl]dimethyl-ammonium salt, [2-((R)-Cyclohexyl-hydroxy-phenyl-methyl)-oxazol-5-ylmethyl]-[2-(3,4-dichloro-benzyloxy)-ethyl]-dimethyl-ammonium salt, [2-(4-Chloro-benzyloxy)-ethyl]-[2-((R)-Cyclohexyl-hydroxy-phenyl-methyl)-oxazol-5-ylmethyl]-dimethyl-ammonium salt, or (R)-1-[2-(4-Fluoro-phenyl)-ethyl]-3-((S)-2-phenyl-2-piperidin-1-yl-propionyloxy)-1-azonia-bicyclo[2.2.2]octane; wherein the counter-ion is, for example, chloride, bromide, sulfate, methanesulfonate, benzenesulfonate (besylate), toluenesulfonate (tosylate), napthalenebissulfonate (napadisylate), phosphate, acetate, citrate, lactate, tartrate, mesylate, maleate, fumarate or succinate).

In a further aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is Oxitropium bromide or Tiotropium bromide.

In one aspect of the invention the muscarinic receptor antagonist is a long acting muscarinic receptor antagonist, that is a muscarinic receptor antagonist with activity that persists for more than 12 hours. Examples of long acting muscarinic receptor antagonists include tiotropium bromide.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide a pharmaceutically acceptable salt thereof (such as the di-D-mandelate salt), and a second active ingredient is Tiotropium bromide.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is Tiotropium bromide.

In yet another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (such as the di-D-mandelate salt), and a second active ingredient is Glycopyrrolate (such as R,R-,R,S-, S,R-, or S,S-glycopyrronium bromide).

In a further aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof (such as the di-D-mandelate salt), and a second active ingredient is (R)-1-[2-(4-Fluoro-phenyl)-ethyl]-3-((S)-2-phenyl-2-piperidin-1-yl-propionyloxy)-1-azonia-bicyclo[2.2.2]octane; wherein the counter-ion is, for example, chloride, bromide, sulfate, methanesulfonate, benzenesulfonate (besylate), toluenesulfonate (tosylate), napthalenebissulfonate (napadisylate), phosphate, acetate, citrate, lactate, tartrate, mesylate, maleate, fumarate or succinate).

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a p38 inhibitor, for example, a compound from WO 2005/042502, 681323, 856553, AMG548 (2-[[(2S)-2-amino-3-phenylpropyl]amino]-3-methyl-5-(2-naphthalenyl)-6-(4-pyridinyl)-4(3H)-pyrimidinone), Array-797, AZD6703, Doramapimod, KC-706, PH 797804, R1503, SC-80036, SCIO469,6-chloro-5-[[(2S,5R)-4-[(4-fluorophenyl)methyl]-2,5-domethyl-1-piperazinyl]carbonyl]-N,N,1-trimethyl-α-oxo-1H-indole-3-acetamide, VX702 or VX745 (5-(2,6-dichlorophenyl)-2-(phenylthio)-6H-pyrimido[1,6-b]pyridazin-6-one).

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a PDE Inhibitor: such as a PDE4 inhibitor {for example, 256066, Arofylline (3-(4-chlorophenyl)-3,7-dihydro-1-propyl-1H-Purine-2,6-dione), AWD 12-281 (N-(3,5-dichloro-4-pyridinyl)-1-[(4-fluorophenyl)methyl]-5-hydroxy-α-oxo-1H-indole-3-acetamide), BAY19-8004 (Bayer), CDC-801 (Calgene), Celgene compound 4(3R)-(3-(3,4-dimethoxyphenyl)-1,3-dihydro-1-oxo-2H-isoindole-2-propanamide), Cilomilast (cis-4-cyano-4-[3-(cyclopentyloxy)-4-methoxyphenyl]-cyclohexanecarboxylic acid), a compound in WO2006098353 from Kyowa Hakko Kogyo Co. Ltd. Japan, 2-(3,5-dichloro-4-pyridinyl)-1-(7-methoxyspiro[1,3-benzodioxole-2,1′-cyclopentan]-4-yl)ethanone (CAS number 185406-34-2)), Compound from Pfizer (2-(3,4-difluorophenoxy)-5-fluoro-N-[cis-4-[(2-hydroxy-5-methylbenzoyl)amino]cyclohexyl]-)-3-pyridinecarboxamide), Compound from Pfizer (2-(3,4-difluorophenoxy)-5-fluoro-N-[cis-4-[[2-hydroxy-5-(hydroxymethyl)benzoyl]amino]cyclohexyl]-3-pyridinecarboxamide,), CT2820, GPD-1116, Ibudilast, IC 485, KF 31334, KW-4490 (Kyowa Hakko Kogyo), Lirimilast ([2-(2,4-dichlorobenzoyl)-6-[(methylsulfonyl)oxy]-3-benzofuranyl p-urea), Merck Compound (N-cyclopropyl-1,4-dihydro-4-oxo-1-[3-(3-pyridinylethynyl)phenyl]-)-1,8-naphthyridine-3-carboxamide), Oglemilast (N-(3,5-dichloro-4-pyridinyl)-4-(difluoromethoxy)-8-[(methylsulfonyl)amino])-1-dibenzofurancarboxamide), ONO6126, ORG 20241 (4-(3,4-dimethoxyphenyl)-N-hydroxy-)-2-thiazolecarboximidamide), PD189659/PD168787 (Parke-Davis), Pentoxifylline (3,7-dihydro-3,7-dimethyl-1-(5-oxohexyl)-)-1H-purine-2,6-dione), Pfizer compound (5-fluoro-N-[4-[(2-hydroxy-4-methyl-benzoyl)amino]cyclohexyl]-2-(thian-4-yloxy)pyridine-3-carboxamide), Pfizer UK 500,001, Piclamilast (3-(cyclopentyloxy)-N-(3,5-dichloro-4-pyridinyl)-4-methoxy-benzamide), PLX-369 (WO 2006026754), Roflumilast (3-(cyclopropylmethoxy)-N-(3,5-dichloro-4-pyridinyl)-4-(difluoromethoxy)benzamide), SCH 351591 (N-(3,5-dichloro-1-oxido-4-pyridinyl)-8-methoxy-2-(trifluoromethyl)-5-quinolinecarboxamide), SelCID™ CC-10004 (Calgene), T-440 (Tanabe), Tetomilast (6-[2-(3,4-diethoxyphenyl)-4-thiazolyl-2-pyridinecarboxylic acid), Tofimilast (9-cyclopentyl-7-ethyl-6,9-dihydro-3-(2-thienyl)-5H-pyrazolo[3,4-c]-1,2,4-triazolo[4,3-a]pyridine), TPI 1100, UCB 101333-3 (N,2-dicyclopropyl-6-(hexahydro-1H-azepin-1-yl)-5-methyl-4-pyrimidinamine), V-11294A (Napp), VM554/VM565 (Vernalis), or Zardaverine (6-[4-(difluoromethoxy)-3-methoxyphenyl]-3(2H)-pyridazinone); or a PDE5 Inhibitor, for example, Gamma-glutamyl[s-(2-iodobenzyl)cysteinyl]glycine, Tadalafil, Vardenafil, sildenafil, 4-phenyl-methylamino-6-chloro-2-(1-imidazolyl)-quinazoline, 4-phenyl-methylamino-6-chloro-2-(3-pyridyl)-quinazoline, 1,3-dimethyl-6-(2-propoxy-5-methanesulphonylamidophenyl)-1,5-dihydropyrazolo[3,4-d]pyrimidin-4-one or 1-cyclopentyl-3-ethyl-6-(3-ethoxy-4-pyridyl)-pyrazolo[3,4-d]pyrimidin-4-one}.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a PDE4 inhibitor, for example, 256066, Arofylline (3-(4-chlorophenyl)-3,7-dihydro-1-propyl-1H-Purine-2,6-dione), AWD 12-281 (N-(3,5-dichloro-4-pyridinyl)-1-[(4-fluorophenyl)methyl]-5-hydroxy-α-oxo-1H-indole-3-acetamide), BAY19-8004 (Bayer), CDC-801 (Calgene), Celgene compound 4(3R)-(3-(3,4-dimethoxyphenyl)-1,3-dihydro-1-oxo-2H-isoindole-2-propanamide), Cilomilast (cis-4-cyano-4-[3-(cyclopentyloxy)-4-methoxyphenyl]-cyclohexanecarboxylic acid), a compound in WO2006098353 from Kyowa Hakko Kogyo Co. Ltd. Japan, 2-(3,5-dichloro-4-pyridinyl)-1-(7-methoxyspiro[1,3-benzodioxole-2,1′-cyclopentan]-4-yl)ethanone (CAS number 185406-34-2)), Compound from Pfizer (2-(3,4-difluorophenoxy)-5-fluoro-N-[cis-4-[(2-hydroxy-5-methylbenzoyl)amino]cyclohexyl]-)-3-pyridinecarboxamide), Compound from Pfizer (2-(3,4-difluorophenoxy)-5-fluoro-N-[cis-4-[[2-hydroxy-5-(hydroxymethyl)benzoyl]amino]cyclohexyl]-3-pyridinecarboxamide,), CT2820, GPD-1116, Ibudilast, IC 485, KF 31334, KW-4490 (Kyowa Hakko Kogyo), Lirimilast ([2-(2,4-dichlorobenzoyl)-6-[(methylsulfonyl)oxy]-3-benzofuranyl p-urea), Merck Compound (N-cyclopropyl-1,4-dihydro-4-oxo-1-[3-(3-pyridinylethynyl)phenyl]-)-1,8-naphthyridine-3-carboxamide), Oglemilast (N-(3,5-dichloro-4-pyridinyl)-4-(difluoromethoxy)-8-[methylsulfonyl)amino]-1-dibenzofurancarboxamide), ONO6126, ORG 20241 (4-(3,4-dimethoxyphenyl)-N-hydroxy-)-2-thiazolecarboximidamide), PD189659/PD168787 (Parke-Davis), Pentoxifylline (3,7-dihydro-3,7-dimethyl-1-(5-oxohexyl)-)-1H-purine-2,6-dione), Pfizer compound (5-fluoro-N-[4-[(2-hydroxy-4-methyl-benzoyl)amino]cyclohexyl]-2-(thian-4-yloxy)pyridine-3-carboxamide), Pfizer UK 500,001, Piclamilast (3-(cyclopentyloxy)-N-(3,5-dichloro-4-pyridinyl)-4-methoxy-benzamide), PLX-369 (WO 2006026754), Roflumilast (3-(cyclopropylmethoxy)-N-(3,5-dichloro-4-pyridinyl)-4-(difluoromethoxy)benzamide), SCH 351591 (N-(3,5-dichloro-1-oxido-4-pyridinyl)-8-methoxy-2-(trifluoromethyl)-5-quinolinecarboxamide), SelCID™ CC-10004 (Calgene), T-440 (Tanabe), Tetomilast (6-[2-(3,4-diethoxyphenyl)-4-thiazolyl-2-pyridinecarboxylic acid), Tofimilast (9-cyclopentyl-7-ethyl-6,9-dihydro-3-(2-thienyl)-5H-pyrazolo[3,4-c]-1,2,4-triazolo[4,3-a]pyridine), TPI 1100, UCB 101333-3 (N,2-dicyclopropyl-6-(hexahydro-1H-azepin-1-yl)-5-methyl-4-pyrimidinamine), V-11294A (Napp), VM554/VM565 (Vernalis), or Zardaverine (6-[4-(difluoromethoxy)-3-methoxyphenyl]-3(2H)-pyridazinone).

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is a PDE4 inhibitor, for example AWD 12-281 (N-(3,5-dichloro-4-pyridinyl)-1-[(4-fluorophenyl)methyl]-5-hydroxy-α-oxo-1H-indole-3-acetamide) or roflumilast.

In another aspect the present invention provides a pharmaceutical product comprising, in combination, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient is roflumilast.

The first active ingredient and the second active ingredient of the pharmaceutical product of the present invention may be administered simultaneously, sequentially or separately to treat respiratory diseases. By simultaneously is meant that the active ingredients are in admixture, or they could be in separate chambers of the same inhaler. By sequential it is meant that the active ingredients are administered, in any order, one immediately after the other. They still have the desired effect if they are administered separately, but when administered in this manner they are generally administered less than 4 hours apart, conveniently less than two hours apart, more conveniently less than 30 minutes apart and most conveniently less than 10 minutes apart, for example less than 10 minutes but not one immediately after the other.

The active ingredients of the present invention may be administered by oral or parenteral (e.g. intravenous, subcutaneous, intramuscular or intraarticular) administration using conventional systemic dosage forms, such as tablets, capsules, pills, powders, aqueous or oily solutions or suspensions, emulsions and sterile injectable aqueous or oily solutions or suspensions. The active ingredients may be delivered to the lung and/or airways via oral administration in the form of a solution, suspension, aerosol or dry powder formulation. These dosage forms will usually include one or more pharmaceutically acceptable ingredients which may be selected, for example, from an adjuvant, carrier, binder, lubricant, diluent, stabilising agent, buffering agent, emulsifying agent, viscosity-regulating agent, surfactant, preservative, flavouring or colorant. As will be understood by those skilled in the art, the most appropriate method of administering the active ingredients is dependent on a number of factors.

In another embodiment the first and second active ingredients are administered via a single pharmaceutical composition (that is, the first and second active ingredients are in admixture). Therefore, the present invention further provides a pharmaceutical composition comprising, in admixture, a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-mandelate salt, and a second active ingredient as defined above. The pharmaceutical composition optionally further comprises a pharmaceutically acceptable adjuvant, diluent or carrier.

The pharmaceutical compositions of the present invention can be prepared by mixing the first active ingredient with the second active ingredient and a pharmaceutically acceptable adjuvant, diluent or carrier. Therefore, in a further aspect of the present invention there is provided a process for the preparation of a pharmaceutical composition, which comprises mixing the first and second active ingredients and a pharmaceutically acceptable adjuvant, diluent or carrier.

It will be understood that the therapeutic dose of each active ingredient administered in accordance with the present invention will vary depending upon the particular active ingredient employed, the mode by which the active ingredient is to be administered, and the condition or disorder to be treated.

In one embodiment of the present invention, the first active ingredient is administered via inhalation. When administered via inhalation the dose of the first active ingredient (that is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide in: salt form, solvate form, or, solvate of salt form) will generally be in the range of from 0.1 microgram (μg) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In one embodiment of the present invention the second active ingredient is administered by inhalation. When administered via inhalation the dose of the second active ingredient will generally be in the range of from 0.1 microgram (,1g) to 5000 μg, 0.1 to 1000 μg, 0.1 to 500 μg, 0.1 to 100 μg, 0.1 to 50 μg, 0.1 to 5 μg, 5 to 5000 μg, 5 to 1000 μg, 5 to 500 μg, 5 to 100 μg, 5 to 50 μg, 5 to 10 μg, 10 to 5000 μg, 10 to 1000 μg, 10 to 500 μg, 10 to 100 μg, 10 to 50 μg, 20 to 5000 μg, 20 to 1000 μg, 20 to 500 μg, 20 to 100 μg, 20 to 50 μg, 50 to 5000 μg, 50 to 1000 μg, 50 to 500 μg, 50 to 100 μg, 100 to 5000 μg, 100 to 1000 μg or 100 to 500 μg. The dose will generally be administered from 1 to 4 times a day, conveniently once or twice a day, and most conveniently once a day.

In another embodiment the present invention provides a pharmaceutical product wherein the molar ratio of first active ingredient to second active ingredient is from 1:1000 to 1000:1, such as from 1:100 to 100:1, for example from 1:50 to 50:1, for example 1:20 to 20:1.

In one embodiment, the present invention provides a pharmaceutical product comprising, in combination, a first active ingredient as defined above, and a second active ingredient as defined above, wherein each active ingredient is formulated for inhaled administration. In a further aspect of this embodiment, the pharmaceutical product is in the form of a pharmaceutical composition comprising the first and second active ingredients in admixture, and which composition is formulated for inhaled administration.

The active ingredients of the present invention are conveniently delivered via oral administration by inhalation to the lung and/or airways in the form of a solution, suspension, aerosol or dry powder (such as an agglomerated or ordered mixture) formulation. For example a metered dose inhaler device may be used to administer the active ingredients, dispersed in a suitable propellant and with or without an additional excipient such as ethanol, a surfactant, lubricant or stabilising agent. A suitable propellant includes a hydrocarbon, chlorofluorocarbon or a hydrofluoroalkane (e.g. heptafluoroalkane) propellant, or a mixture of any such propellants, for example in a pressurised metered dose inhaler (pMDI). Preferred propellants are P134a and P227, each of which may be used alone or in combination with other another propellant and/or surfactant and/or other excipient. A nebulised aqueous suspension or, preferably, solution may also be employed, with or without a suitable pH and/or tonicity adjustment, either as a unit-dose or multi-dose formulation. A suitable device for delivering a dry powder is Turbuhaler®.

The pharmaceutical product of the present invention can, for example, be administered: via an inhaler having the first and second active ingredients in separate chambers of the inhaler such that on administration the active ingredients mix in either the mouthpiece of the inhaler or the mouth of a patient or both (for simultaneous use); or, where the first and second active ingredients are in separate inhalers, via separate inhalers (for separate or sequential use); or the first and second active ingredients are in admixture in an inhaler when the inhaler is supplied to a patient (for simultaneous use).

A dry powder inhaler may be used to administer the active ingredients, alone or in combination with a pharmaceutically acceptable carrier (such as lactose), in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices is available.

The combination of the present invention may be used to treat diseases of the respiratory tract such as obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

Accordingly, the present invention further provides a pharmaceutical product according to the invention for simultaneous, sequential or separate use in therapy.

The present invention further provides the use of a pharmaceutical product according to the invention in the manufacture of a medicament for the treatment of a respiratory disease, in particular chronic obstructive pulmonary disease, asthma, rhinitis, emphysema or bronchitis (such as chronic obstructive pulmonary disease or asthma; for example chronic obstructive pulmonary disease).

The present invention still further provides a method of treating a respiratory disease which comprises simultaneously, sequentially or separately administering:

(a) a therapeutically effective dose of a first active ingredient as defined above; and,
(b) a therapeutically effective dose of a second active ingredient as defined above;
to a patient in need thereof.

In a further aspect the present invention provides the use of a pharmaceutical product, kit or composition as hereinbefore described for the treatment of a respiratory disease, in particular chronic obstructive pulmonary disease, asthma, rhinitis, emphysema or bronchitis (such as chronic obstructive pulmonary disease or asthma; for example chronic obstructive pulmonary disease).

In the context of the present specification, the term “therapy” also includes “prophylaxis” unless there are specific indications to the contrary. The terms “therapeutic” and “therapeutically” should be construed accordingly. Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

In another aspect the present invention provides a salt of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide, wherein the salt is a salt formed with an acid selected from the group comprising: naphthalene-2-sulfonic acid, hippuric acid, sulfuric acid, 4-methylbenzenesulfonic acid, naphthalene-1,5-disulfonic acid, benzenesulfonic acid, methanesulfonic acid, maleic acid and saccharin.

A salt of the present invention, as hereinbefore defined, may be used to treat a disease of the respiratory tract such as an obstructive disease of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

Accordingly, the present invention further provides a salt according to the invention, as hereinbefore defined, for use in therapy.

The present invention further provides the use of a salt according to the invention, as hereinbefore defined, in the manufacture of a medicament for the treatment of a respiratory disease, in particular chronic obstructive pulmonary disease, asthma, rhinitis, emphysema or bronchitis (such as chronic obstructive pulmonary disease or asthma; for example chronic obstructive pulmonary disease).

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a salt of the invention, as hereinbefore defined.

For the above-mentioned therapeutic uses the dosage administered will, of course, vary with the compound employed, the mode of administration, the treatment desired and the disorder indicated. For example, the daily dosage of a salt of the invention, as hereinbefore defined, if inhaled, may be in the range from 0.05 micrograms per kilogram body weight (μg/kg) to 100 micrograms per kilogram body weight (μg/kg). Alternatively, if the salt of the invention is administered orally, then the daily dosage of the compound of the invention may be in the range from 0.01 micrograms per kilogram body weight (μg/kg) to 100 milligrams per kilogram body weight (mg/kg).

A salt of the invention, as hereinbefore defined, may be used on its own but will generally be administered in the form of a pharmaceutical composition in which a salt of the invention, as hereinbefore defined, (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will for example comprise from 0.05 to 99% w (percent by weight), such as from 0.05 to 80% w, for example from 0.10 to 70% w, and such as from 0.10 to 50% w, of active ingredient, all percentages by weight being based on total composition.

The present invention also provides a pharmaceutical composition comprising a salt as hereinbefore defined, in association with a pharmaceutically acceptable adjuvant, diluent or carrier.

The invention further provides a process for the preparation of a pharmaceutical composition of the invention which comprises mixing a salt of the invention, as hereinbefore defined, with a pharmaceutically acceptable adjuvant, diluent or carrier.

A pharmaceutical composition of the invention can be administered topically (e.g. to the skin or to the lung and/or airways) in the form, e.g., of a cream, solution, suspension, heptafluoroalkane (HFA) aerosol or dry powder formulation, for example, a formulation in the inhaler device known as the Turbuhaler®; or systemically, e.g. by oral administration in the form of a tablet, capsule, syrup, powder or granule; or by parenteral administration in the form of a solution or suspension; or by subcutaneous administration; or by rectal administration in the form of a suppository; or transdermally.

A dry powder formulation or pressurized HFA aerosol of a salt of the invention, as hereinbefore defined, may be administered by oral or nasal inhalation. For inhalation, the salt is desirably finely divided. The finely divided salt has, for example, a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

A salt of the invention, as hereinbefore defined, may also be administered by means of a dry powder inhaler. The inhaler may be a single or a multi dose inhaler, and may be a breath actuated dry powder inhaler.

One possibility is to mix the finely divided salt of the invention, as hereinbefore defined, with a carrier substance, for example, a mono-, di- or polysaccharide, a sugar alcohol, or another polyol. A suitable carrier is, for example, a sugar, for example, lactose, glucose, raffinose, melezitose, lactitol, maltitol, trehalose, sucrose, mannitol; or starch. Alternatively the finely divided salt may be coated by another substance. The powder mixture may also be dispensed into hard gelatine capsules, each containing the desired dose of the active ingredient.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

A salt of the invention, as hereinbefore defined, may also be administered in conjunction with another compound used for the treatment of one or more of the above conditions.

The invention therefore further relates to a combination therapy wherein a salt of the invention, as hereinbefore defined, or a pharmaceutical composition or formulation comprising a salt of the invention, is administered concurrently or sequentially or as a combined preparation with another therapeutic agent or agents, for the treatment of one or more of the conditions listed.

In yet another aspect the present invention provides an intermediate dicyclohexylammonium N-[benzyloxy)carbonyl]-N-[2-(3-fluorophenyl)ethyl]-β-alaninoate:

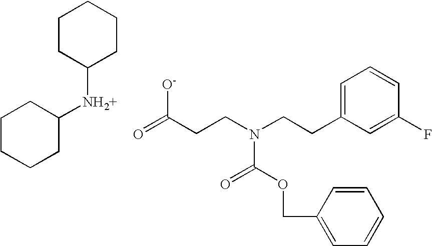

N-[(benzyloxy)carbonyl]-N-[2-(3-fluorophenyl)ethyl]-β-alaninoate is an important intermediate in the preparation of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide, or a pharmaceutically acceptable salt thereof, and the dicyclohexylammonium salt of N-[(benzyloxy)carbonyl]-N-[2-(3-fluorophenyl)ethyl]-β-alaninoate is crystalline. This allows for easy and effective purification mid-way through the process.

General Preparative Methods.

Unless stated otherwise, starting materials were commercially available, all solvents and commercial reagents were of laboratory grade and were used as received, and, where appropriate, reactions were carried out under an atmosphere of an inert gas such as nitrogen. Quoted temperatures refer to applied temperatures unless otherwise indicated as being internal temperature. Ambient temperature refers to a temperature in the range 17 to 28° C. Concentration of solutions was carried out by evaporation under reduced pressure (in vacuo), e.g. using a Büchi Rotavapor® rotary evaporator, unless otherwise stated.

Thin Layer Chromatography (TLC) was carried out using aluminium- or glass-backed plates coated with silica (particle size <63 μm; porosity 60 Å; surface area ˜500 m²/g), with a fluorescent (UV₂₅₄) indicator. Following elution, the plates were visualized by either UV₂₅₄ irradiation, or development with a suitable indicator, such as iodine (pre-absorbed onto silica), an aqueous solution of potassium permanganate, or an aqueous solution of cerium (IV) ammonium nitrate. Examples of indicator preparations can be found in ‘Experimental Organic Chemistry: Preparative and Microscale’ 2^{nd} Ed. (Harwood, L., Moody, C. and Percy, J.), WileyBlackwell, 1998.

Analytical HPLC was carried out using either a Waters XBridge™ C8 3.5 μm column eluting with a gradient of acetonitrile in either 0.1% aqueous trifluoroacetic acid, 0.1% aqueous formic acid, 0.1% aqueous ammonium acetate or 0.1% aqueous ammonia; a Waters XBridge™ C18 3.5 μm column with a gradient of acetonitrile in 0.1% aqueous ammonia; a Waters Symmetry™ C18 3.5 μm column with a gradient of acetonitrile in 0.1% aqueous trifluoroacetic acid; a Waters Sunfire™ C8 3.5 μm column with a gradient of acetonitrile in 0.1% aqueous trifluoroacetic acid; or a Phenomenex Gemini™ C18 3 μm column with a gradient of acetonitrile in 0.1% aqueous trifluoroacetic acid. UV spectra of the eluted peaks were measured using a diode array on an Agilant 1100® system, or equivalent.

Medium pressure liquid chromatography (MPLC) on silica (particle size <63 μm; porosity 60 Å; surface area ˜500 m²/g) was carried out using pre-packed Biotage FLASH™ columns or equivalent, e.g. Thomson SINGLE StEP™, Biotage Isolute™, Teledyne Isco RediSep™, or Silicycle UltraPure silica columns at recommended solvent flow rates and sample loadings. Fraction purity was determined by either TLC or analytical HPLC.

Preparative HPLC was carried out using either a Phenomenex Gemini™ C18 5 μm column, a Waters Sunfire™ C18 5 μm column, a Waters XBridge™ C8 5 μm column or a Waters XTerra™ 5 μm, unless otherwise detailed, using either acetonitrile in aqueous 0.1-0.2% trifluoroacetic acid, acetonitrile in aqueous 0.1-0.2% ammonium acetate, or acetonitrile in an aqueous 0.1-0.2% ammonia solution as eluent, as detailed. Fractions were collected following detection by UV spectroscopy at a wavelength such as 220 or 254 nm. Fraction purity was determined by either TLC or analytical HPLC.

¹H NMR spectra were recorded on a Varian UnityInova 500 MHz, 400 MHz or 300 MHz instrument or a Bruker DPX 300 (300 MHz). Either the central peaks of chloroform-d (CDCl₃; δ_{H} 7.27 ppm), dimethylsulfoxide-d₆ (d₆-DMSO; δ_{H} 2.50 ppm) or methanol-d₄ (CD₃OD; δ_{H} 3.31 ppm), or an internal standard of tetramethylsilane (TMS; δ_{H} 0.00 ppm) were used as references. Mass spectra were recorded on an Agilent MSD (+ve and −ve APCI and/or electrospray (e.g. in multimode)) following analytical HPLC.

XRPD was carried out on PANalytical CubiX PRO machine in Ø-Ø configuration over the scan range 2° to 40° 2Ø with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.

DSC thermograms were measured using a TA Q1000 Differential Scanning Calorimeter, with aluminium pans and pierced lids. The sample weights varied between 0.3 to 5 mg. The procedure was carried out under a flow of nitrogen gas (50 ml/min) and the temperature studied from 25 to 300° C. at a constant rate of temperature increase of 10° C. per minute.

All other processes were carried out using standard laboratory techniques, e.g. as detailed in ‘Experimental Organic Chemistry: Preparative and Microscale’ 2^{nd} Ed. (Harwood, L., Moody, C. and Percy, J.), WileyBlackwell, 1998.

The abbreviations or terms used in the preparations have the following meanings:

SCX: Solid phase extraction with a sulfonic acid sorbent

HPLC: High performance liquid chromatography

DMF: N,N-Dimethylformamide

THF: Tetrahydrofuran

NMP: N-Methylpyrrolidinone

HATU: O-(7-Azabenzotriazol-1-yl)-N,N,N′,N′-tetramethyluronium hexafluorophosphate

TFA: Trifluoroacetic acid

TBME: tent-Butyl(methyl)ether

IMS: Industrial Methylated Spirits

CBZ Carbonyloxybenzyl

### Preparation 1.N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide bis-trifluoroacetic acid salt

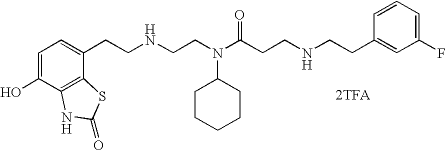

### i) Benzyl (2,2-dimethoxyethyl)[2-(4-hydroxy-2-oxo-2,3-dihydro-1,3-benzothiazol-7-yl)ethyl]carbamate

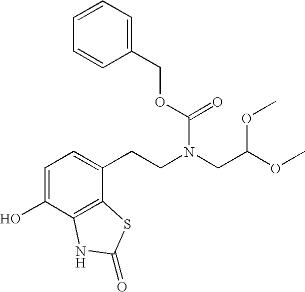

7-(2-Aminoethyl)-4-hydroxy-1,3-benzothiazol-2(3H)-one hydrobromide (20g) was dissolved in a mixture of THF (300 mL) and water (150 mL). Sodium hydrogen carbonate (5.77 g) was added and the mixture stirred for 15 min. Acetic acid (7.86 mL) was added, followed by dimethoxyacetaldehyde (14.9 g, 12.91 mL) and the mixture stirred for a further 30 min. Sodium cyanoborohydride (8.64 g) was added portion-wise over 10 min and the solution stirred for a further 20 h. Ethyl acetate (500 mL) and a solution of sodium hydrogen carbonate (17.33 g) in water (250 mL) were added, the mixture was stirred vigorously, benzyl chloroformate (8.78 g, 7.35 mL) was added, and the mixture stirred for 2 h. The organic layer was separated, washed with water, 0.1M aq. HCl, water and brine, dried (anhydrous Na₂SO₄), filtered and evaporated. The resulting material was purified by flash chromatography on silica using 10% methanol in dichloromethane as eluent to give the sub-title compound as a light brown gum (23.1 g). ¹H NMR δ_{(DMSO)} 11.60 (1H, s), 9.90 (1H, s), 7.39-7.12 (5H, m), 6.73 (2H, m), 5.05 (2H, m), 4.43 (0.5H, t), 4.35 (0.5H, t), 3.41 (2H, m), 3.33 (1.5H, s), 3.27 (3H, s), 3.22 (1.5H, s), 3.19 (2H, m), 2.69 (2H, q).

MS (APCI+) 433 [M+H]⁺

### ii) Benzyl[2-(4-hydroxy-2-oxo-2,3-dihydro-1,3-benzothiazol-7-yl)ethyl](2-oxoethyl)carbamate

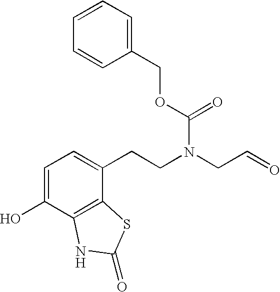

The acetal from step i) (5g) was dissolved in acetone (100 mL), 2M HCl in dioxane (50 mL) was added and the mixture stirred for 3 h. Concentrated HCl (2 mL) was added and mixture stirred for a further 20 h. Toluene (100 mL) was added and the solvents removed in vacuo. The residue was dissolved in THF (200 mL), toluene (100 mL) added, and the solvents removed in vacuo (x2) to give the sub-title compound as an off white solid (4.5 g). ¹H NMR δ_{(DMSO)} 11.61 (1H, m), 9.91 (1H, m), 9.41 (1H, s), 7.31 (5H, m), 6.74 (2H, m), 5.01 (2H, m), 4.04 (2H, d), 3.46 (2H, t), 2.69 (2H, t).

MS (APCI+) 387 [M+H]⁺

### iii) Benzyl [2-(cyclohexylamino)ethyl][2-(4-hydroxy-2-oxo-2,3-dihydro-1,3-benzothiazol-7-yl)ethyl]carbamate

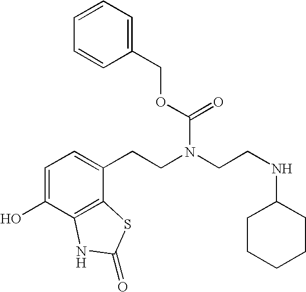

The product of step iii) (0.5 g) was added to a solution of cyclohexylamine (0.23 g, 0.33 mL) in a mixture of THF (10 mL) and water (1 mL) and the mixture was stirred for 15 min. Sodium cyanoborohydride (0.17 g) was added, followed by acetic acid (0.24 g, 0.23 mL) and the reaction stirred for a further 2 h. The reaction was quenched with saturated aqueous sodium hydrogen carbonate, extracted with ethyl acetate, washed with brine, dried (anhydrous Na₂SO₄), filtered and evaporated to give the sub-title compound as a light brown gum (0.6 g). ¹H NMR 90° C. δ_{(DMSO)} 7.40-7.50 (m, 5H), 6.86 (d, 1H), 6.80 (d, 1H), 5.18 (s, 2H), 3.72 (t, 2H), 3.56 (t, 2H), 2.94 (t, 2H), 2.83 (t, 2H), 1.96 (m, 2H), 1.84 (m, 4H), 1.68 (m, 1H), 1.29 (m, 4H).

MS (APCI+) 470 [M+H]⁺

### iv) Benzyl {2-[acryloyl(cyclohexyl)amino]ethyl}[2-(4-hydroxy-2-oxo-2,3-dihydro-1,3-benzothiazol-7-yl)ethyl]carbamate

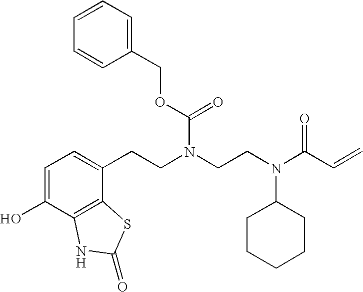

The amine as prepared in step iii) (1.57 g) was dissolved in dichloromethane (20 mL), chlorotrimethylsilane (1.29 mL) and triethylamine (1.91 mL) were added, and the mixture was stirred at room temperature for 1 h. The mixture was cooled to 0° C., acryloyl chloride (336 ul) added, and the mixture was stirred, warming to room temperature, for 3 h. The reaction mixture was diluted with dichloromethane, washed with saturated sodium hydrogen carbonate, then with water, dried (anhydrous Na₂SO₄), filtered and evaporated. The residue was purified by flash chromatography on silica using ethyl acetate (30, 50, 70, 100%) in isohexane as eluent to give the sub-title compound (1.1 g). MS (APCI+) 524 [M+H]⁺

### v) N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide bis-trifluoroacetic acid salt

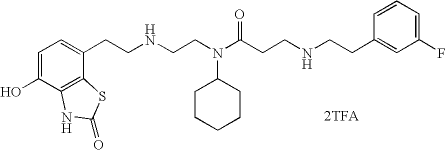

The acrylamide as prepared in step iv) (1 mL of a 0.33M solution in ethanol) was treated with 3-fluorophenethylamine (97 ul) and the mixture was stirred at 50° C. for 18 h. The product was purified by SCX chromatography eluting with 1N ammonia in methanol. The solvents were removed in vacuo and the residue was re-dissolved in dichloromethane (0.5 mL). This solution was cooled in an ice/water bath, hydrogen bromide 30 wt % solution in acetic acid (0.5 mL) was added, and the mixture was stirred at room temperature for 2 h. Toluene (1 mL) was added to the reaction and all solvents were removed in vacuo. The residue was azeotroped with toluene, then ethanol (x2) before being purified by reverse phase HPLC (5-40% acetonitrile in aqueous TFA). The residue was triturated with diethyl ether to give the title compound as a white solid (30 mg). ¹H NMR δ_{(DMSO)} 11.73 (1H, s), 10.13 (1H, s), 8.84-8.48 (4H, m), 7.43-7.34 (1H, m), 7.18-7.08 (3H, m), 6.86 (1H, d), 6.75 (1H, d), 3.59-3.45 (4H, m), 3.30-3.10 (5H, m), 3.03-2.93 (4H, m), 2.85-2.77 (4H, m), 1.81-1.03 (10H, m). MS (Multimode+) 529 [(M-salt)+H]⁺

### Preparation 2.N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt

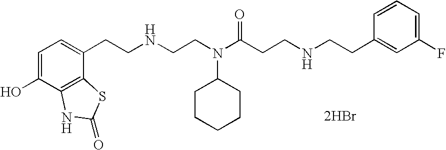

### tert-Butyl N-[2-(3-fluorophenyl)ethyl]-β-alaninate

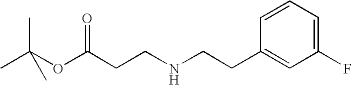

tert-Butyl acrylate (5.61 mL) was added to a solution of 3-fluorophenethylamine (5.0 mL) in ethanol (200 mL) and the mixture stirred at room temperature for 2 days. The solvent was removed in vacuo to afford the sub-title compound as an oil (9.6 g). ¹H NMR δ_{(CDC13)} δ 7.28-7.20 (m, 1H), 7.01-6.85 (m, 3H), 2.88-2.74 (m, 6H), 2.41 (t, J=6.5 Hz, 2H), 1.42 (s, 9H).

MS (APCI⁺) 268 [M+H]⁺

### ii) tert-Butyl N-[(benzyloxy)carbonyl]-N-[2-(3-fluorophenyl)ethyl]-β-alaninate

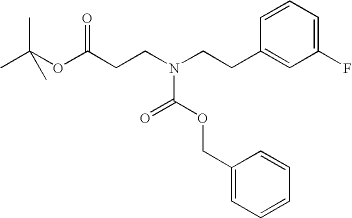

Benzyl chloroformate (5.57 mL) was added dropwise over 5 minutes to a solution of tert-butyl N-[2-(3-fluorophenyl)ethyl]-β-alaninate, as prepared in step i) (9.5 g) and triethylamine (5.94 mL) in dichloromethane (100 mL) at −5° C. The reaction was allowed to attain room temperature and stirred overnight. The solvent was removed in vacuo and the residue purified by flash chromatography on silica using 10% ethyl acetate in isohexane as eluent to give the sub-title compound as an oil (11.5 g). ¹H NMR (DMSO) δ 7.37-7.22 (m, 5H), 7.06-6.91 (m, 3H), 5.05 (s, 2H), 3.46 (t, J=7.3 Hz, 2H), 3.39 (t, J=7.0 Hz, 2H), 2.81 (t, J=7.4 Hz, 2H), 2.41 (t, J=7.2 Hz, 2H), 1.38 (s, 9H). MS (APCI⁺) 402 [M+H]⁺

### iii) N-[(Benzyloxy)carbonyl]-N-[2-(3-fluorophenyl)ethyl]-β-alanine

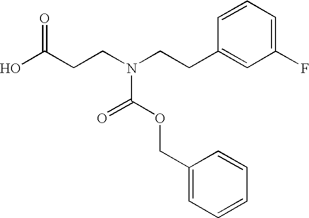

Trifluoroacetic acid (50 mL) was added to a solution of the tert-butyl ester, as prepared in step ii) (11.5 g) in dichloromethane (50 mL) and the mixture was stirred at room temperature for 2 h. The solvent was removed in vacuo and the oil azeotroped with toluene (x2) to afford the sub-title compound as a viscous oil (10.5 g). ¹H NMR δ_{(DMSO)} δ7.41-7.27 (m, 6H), 7.08-6.94 (m, 3H), 5.04 (d, J=25.9 Hz, 2H), 3.45 (t, J=7.4 Hz, 2H), 3.38 (s, 2H), 2.84-2.76 (m, 2H), 2.45 (t, J=7.0 Hz, 2H). MS (APCI⁻) 344 [M−H]⁻

### iv) Benzyl {3-[cyclohexyl(2,2-dimethoxyethyl)amino]-3-oxopropyl}[2-(3-fluorophenyl)ethyl]carbamate

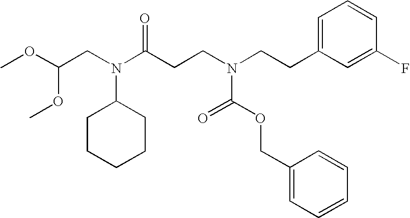

To a solution of N-[(benzyloxy)carbonyl]-N-[2-(3-fluorophenyl)ethyl]-β-alanine, as prepared in step iii) (5 g) dissolved in dichloromethane (50 mL) with stirring under nitrogen was added dimethylformamide (2 drops) followed by oxalyl chloride (1.64 mL) dropwise over 10 min. The mixture was stirred at room temperature for 1 h, concentrated in vacuo and redissolved in dichloromethane (25 mL). The solution was added dropwise to a preformed mixture of N-(2,2-dimethoxyethyl)cyclohexanamine (2.71 g) and triethylamine (3.0 mL) in dichloromethane (25 mL) at 0° C. under nitrogen. The mixture was stirred at 0° C. for 1 h, then water (25 mL) was added and the layers were separated. The organic layer was washed with 2M hydrochloric acid, saturated aqueous sodium bicarbonate and brine before being dried (anhydrous MgSO₄) filtered and concentrated in vacuo to give the sub-title compound as an oil (7.45 g). ¹H NMR δ_{(DMSO)} 7.35 (5H, s), 7.25-7.15 (1H, m), 7.02-6.76 (3H, m), 5.12 (2H, d), 4.62-4.52 (1H, m), 4.39-4.26 (0.5H, m), 4.23-4.09 (0.5H, m), 3.59-3.46 (4H, m), 3.38 (6H, s), 3.35-3.23 (2H, m), 2.92-2.45 (4H, m), 1.88-0.99 (10H, m)

MS: APCI (+ve): 515 [M+H]⁺

### v) Benzyl {3-[cyclohexyl(2-{[2-(4-hydroxy-2-oxo-2,3-dihydro-1,3-benzothiazol-7-yl)ethyl]amino}ethyl)amino]-3-oxopropyl}[2-(3-fluorophenyl)ethyl]carbamate

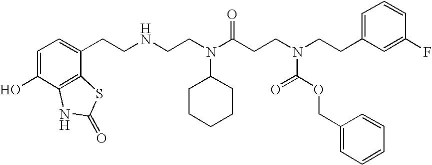

para-Toluenesulfonic acid monohydrate (10.4 g) was added to a solution of the product from step i) (9.4 g) in dichloromethane (94 mL). The mixture was stirred at room temperature for 40 min and a solution of saturated aqueous sodium bicarbonate (4.6 g) in water (100 mL) was added. The layers were separated and the organic phase was washed with saturated aqueous sodium bicarbonate (50 mL) and water (50 mL) before being dried (anhydrous MgSO₄), filtered and concentrated. The resulting oil was redissolved in N-methylpyrrolidinone (30 mL) and added to a solution of 7-(2-aminoethyl)-4-hydroxy-1,3-benzothiazol-2(3H)-one hydrobromide (6.0 g) and triethylamine (2.9 mL) in N-methylpyrrolidinone (30 mL) and water (3 mL). Sodium triacetoxyborohydride (6.0 g) was added and the mixture was stirred at room temperature for 3 h before being poured into water (600 mL) and extracted with ethyl acetate (2×150 mL). The organic layer was washed with aqueous sodium chloride (100 mL). A solid precipitated from the organic layer, which was partially concentrated in vacuo, and the precipitate was collected by filtration and washed with ethyl acetate to give the sub-title compound as a colourless solid (7.7 g). ¹H NMR δ_{(DMSO)} δ 7.41-7.24 (5H, m), 7.10-6.93 (3H, m), 6.86 (1H, d), 6.77 (1H, m), 5.05 (2H, d), 3.63-3.26 (8H, m), 3.13-3.01 (2H, m), 2.99-2.76 (6H, m), 2.62-2.52 (1H, m), 1.79-0.95 (10H, m) MS: APCI (+ve): 663 [M+H]⁺

### vi) N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide dihydrobromide salt

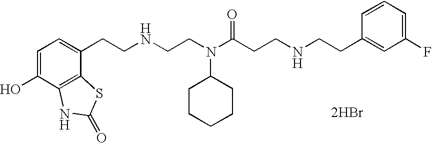

To a solution of the product from step v) (1g) in acetic acid (3 mL) stirred at room temperature was added hydrobromic acid in acetic acid (33%, 3 mL). The mixture was stirred for 80 min and then tert-butyl methyl ether (8 mL) was added. The mixture was stirred for 5 min and then filtered, washing with tert-butyl methyl ether (8 mL). Purification by recrystallisation from hot ethanol (20 mL) gave the title compound (0.82 g) as a solid. ¹H NMR δ_{(DMSO)} 11.72 (1H, s), 10.08 (1H, s), 8.60 (4H, s), 7.39 (1H, q), 7.22-7.03 (3H, m), 6.88 (1H, d), 6.81-6.72 (1H, m), 3.65-3.47 (3H, m), 3.32-3.08 (6H, m), 3.07-2.95 (4H, m), 2.94-2.81 (4H, m), 1.76 (3H, t), 1.68-1.22 (5H, m), 1.19-1.02 (2H, m) MS: APCI (+ve): 529 [M+H]⁺

Preparation 2 (Form C).N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form C). A solution of hydrogen bromide in acetic acid (33%, 12.9 mL) was added to a stirred solution of the product from preparation 2, step v) (3.23 g) in acetic acid (19.4 mL) in an ambient temperature water bath. The mixture was stirred for 2.5 h and then diluted slowly with 1:1 mixture of diethyl ether and ethyl acetate (230 mL) causing precipitation. The mixture was stirred vigourously for 90 min to give a fine precipitate, which was isolated by filtration. The residue was washed with a a 1:1 mixture of diethyl ether and ethyl acetate and then dried by a stream of air, then in vacuo for 1 h, to afford a peach coloured solid (3.09 g). The peach coloured solid (3.09 g) was dissolved in ethanol (75 mL) with some sonication and the solution was left to stand at ambient temperature for 2 h, causing precipitation of a white solid. The mixture was stirred for 18 h and the white solid was isolated by filtration. The residue was washed with ethanol (40 mL), then dried by a stream of air, then in vacuo for 4 h, to afford the title compound (2.25 g). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form C) is presented in FIG. 1.

Preparation 2 (Form D). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form D). A sample of Preparation 2 (Form C) (10 mg) was dissolved in hot mixture of acetonitrile (1.5 mL) and water (0.05 mL). The solution was allowed to cool to ambient temperature, causing precipitation of a white solid. The supernatant was decanted away and the residue was washed with acetonitrile and dried in vacuo. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form D) is presented in FIG. 2.

Preparation 2 (Form E). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form E). A sample of Preparation 2 (Form C) (10 mg) was partly dissolved in water (0.5 mL). The suspension was stirred continuously for 6 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form E) is presented in FIG. 3.

Preparation 2 (Form F). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form F). A sample of Preparation 2 (Form C) (10 mg) was partly dissolved in 1,4-dioxane (0.5 mL). The suspension was stirred continuously for 6 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form F) is presented in FIG. 4.

Preparation 2 (Form G). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form G). A sample of Preparation 2 (Form C) (10 mg) was dissolved in hot mixture of acetone (1 mL) and water (0.05 mL). The solution was allowed to cool to ambient temperature and stood for 1 week, causing precipitation of a white solid. The supernatant was decanted away and the residue was washed with acetone and dried in vacuo. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form G) is presented in FIG. 5.

Preparation 2 (Form H). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form H).

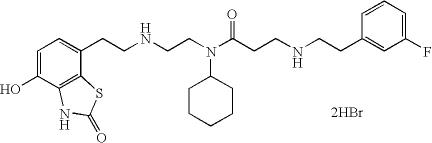

### i) N-(2,2-dimethoxyethyl)cyclohexanamine

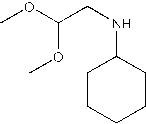

To heated (130° C.) cyclohexylamine (82g) was added chloroacetaldehyde dimethylacetal (40g) over 30 minutes followed by rinse with cyclohexylamine (3g). The mixture was stirred at 120-138° C. overnight. The mixture was cooled to 20° C. and quenched with 20% sodium hydroxide (93.7 g), separated and the organic layer vacuum distilled at an applied temperature of 120-145° C. and a vacuum of 15-21 mbar giving a head temperature of 100-120° C., to afford the sub-title compound (54.2 g).

### ii) tert-Butyl N-[(benzyloxy)carbonyl]-N-[2-(3-fluorophenyl)ethyl]-β-alaninate

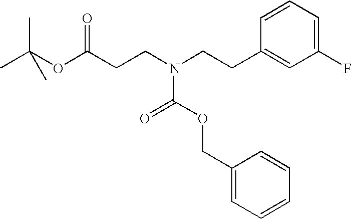

To a heated (reflux 74-75° C.) solution of 3-(fluorophenyl)ethylamine (45.0 Kg) in IMS (131.5 kg), was added tent-butyl acrylate (43.7 Kg) over 45 min, followed by an IMS rinse (17.0 Kg) and the resultant solution stirred for 2 h 45 min at reflux. The reaction was cooled to −5° C. Triethylamine (39.5 Kg) was added over 90 min, resulting in a mild exotherm, followed by an IMS rinse (3 Kg). Benzyl chloroformate (57 Kg) was added over 16 h at −5.9° C. to −1.9° C., followed by an IMS rinse (3 Kg). The reaction was maintained at −5 to 0° C. for 1 h. The reaction was warmed to 20-25° C. 2-methyltetrahydrofuran (186 Kg), water (131 Kg) and 5% citric acid solution (43.5 Kg) were added, and separated. The aqueous layer was back-extracted with 2-methyltetrahydrofuran (112 Kg). The organic layer was washed with a combination of water (175 Kg), 5% citric acid solution (44 Kg) and 20% sodium chloride solution (66 Kg). The combined organic layers were washed with 20% sodium chloride solution (131 Kg). The organic layer was distilled at a internal temperature of 23.1-54.5° C. at 200-15 mbar. To the residue was charged 2-methyletrahydrofuran (42 kg) and this solution distilled under vacuum at internal temperature of 50.1-55.5° C. at 100-15 mbar. To this residue was charged 2-methyltetrahydrofuran (50 kg). A final vacuum distillation was performed at a internal temperature of 50.1-52.1° C. at 200-15 mbar. This gave 136 kg of crude oil which by GC analysis showed ethanol content to be 0.05%. The oil was dissolved in 2-methyltetrahydrofuran (576 kg) ready for the next stage. GC Method: Perkin Elmer fitted with a Zebron ZB5 30 m. Injector: 275° C. Detector: 300° C. Carrier: Nitrogen at 10 psi. Method: 50° C. for 5 min then up to 280° C. at an increment of 10° C./min.

### iii) N-[(Benzyloxy)carbonyl]-N-[2-(3-fluorophenyl)ethyl]-(3-alanine

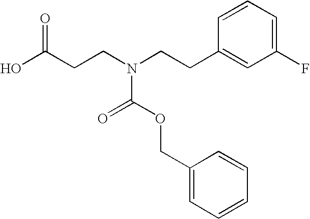

To the solution from Preparation 2 (Form H) Step ii) (136 kg) in 2-methyltetrahydrofuran (576 kg) was added 85% phosphoric acid (197 kg over 1 h at 9.9 to 25.3° C. followed by a line rinse of 2-methyltetrahydrofuran (10 kg). The reaction was heated to reflux for 76 h. The reaction was cooled to <25° C. and quenched with 32% sodium hydroxide (307 kg) over 4 min at 16.1 to 27.6° C. 2-Methyltetrahydrofuran (167 kg) followed by water (547 kg) was charged. The pH was adjusted to 6.6-6.8 by addition of 32% sodium hydroxide (90 kg) and the lower aqueous layer was discarded. The organic layer was then diluted with TBME (506 kg) followed by 1M sodium hydroxide solution (763.5 kg). The lower aqueous layer was removed and retained at 30° C. to dissolve all solids. The aqueous layer was washed with 2-methyl tetrahydrofuran (750 kg) and 36% hydrochloric acid (175 kg). Following separation, the organic layer was diluted with further 2-methyltetrahydrofuran (583 kg) and the vessel configured for vacuum distillation at a internal temperature of 28.3-39.9° C. at 340-250 mbar. 1198 L of 2-methyltetrahydrofuran was removed in this procedure. The mixture was cooled <40° C. and re-diluted with fresh 2-methyltetrahydrofuran (1000 kg) and then re-distilled at internal temperature of 27.0-43.5° C. under 300-250 mbar. At this point the organic mixture gave a Karl-Fischer analysis result of 0.01 wt % water. The mixture was cooled to 25-30° C. and the organic solution of product (80.5 kg in 2-methyltetrahydrofuran 345.5 kg); confirmed by HPLC assay against a known standard of the title compound) was in the subsequent stage. KF method: Mettler Toledo Model DL31; Hydranal Composite 5K and Hydranol Methanol Dry

HPLC method details:

Column: Waters Atlantis T3; Dimensions: 50 mm×3 mm×3 μm.
Mobile phase “A”: 0.03% TFA in water;
Mobile phase “B”: 0.03% TFA in acetonitrile;
Flow Rate: 1.5 ml/min;

### Elution Gradient:

| Time, min | % “B” |
|---|---|
| 0 | 0 |
| 15 | 60 |
| 20 | 60 |

### Detection: UV;

Detector Wavelength: 220 nm, bandwidth 7 nm, reference 360 nm, bandwidth 100 nm;

### Injection Volume: 3 μl;

### Column Temperature: 40° C.

Retention time of N-[(Benzyloxy)carbonyl]-N-[2-(3-fluorophenyl)ethyl]-β-alanine: 14.44 mins.

### iv) Benzyl {3-[cyclohexyl(2,2-dimethoxyethyl)amino]-3-oxopropyl}[2-(3-fluorophenyl)ethyl]carbamate

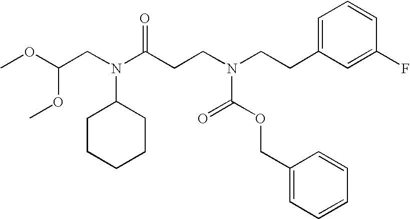

To the solution of Preparation 2 (Form H) Step iii) (CBz Acid) (80.5 Kg) in 2-methyltetrahydrofuran (345.5 kg; KF of solution=0.04%) was charged the material from
Preparation 2 (Form H) Step i) (47 Kg) over 40 min (resulting in an exotherm from 13.8° C. to 16.3° C.) followed by a 2-methyltetrahydrofuran rinse (10 Kg). Triethylamine (79.5 Kg) was charged over 1 h 10 min, followed by a 2-methyltetrahydrofuran rinse (5 Kg). The reaction contents were cooled to 10° C. 2,4,6-tripropyl-1,3,5,2,4,6-trioxatriphosphorinane-2,4,6-trioxide (T3P) (260 Kg) was charged over 6 h 20 min (resulting in an exotherm from 11.0° C. to 19.0° C.) followed by a 2-methyltetrahydrofuran rinse (21 Kg). The reaction was maintained at 19° C. for 1 h and quenched with 0.5 M sodium bicarbonate solution (619 Kg) over 3 h 5 min (resulting in an exotherm from 20.1° C. to 26.1° C.), and separated. The organic layer was washed with 20% sodium chloride (458 Kg) and 20% sodium chloride (451 Kg). The organic layer was concentrated under vacuum with internal temperature of 16.1-37.5° C. at 500 to 150 mbar affording a 2-methyltetrahydrofuran solution of the sub-title compound (119.5 Kg in 73.5 Kg 2-methyltetrahydrofuran, i.e. a 61.9 wt % solution as determined by ¹⁹F NMR assay against 1,2-difluorobenzene, using a Bruker DPX 300 spectrometer in CDCl₃.

### v) Benzyl {3-[cyclohexyl(2-{[2-(4-hydroxy-2-oxo-2,3-dihydro-1,3-benzothiazol-7-yl)ethyl]amino}ethyl)amino]-3-oxopropyl}[2-(3-fluorophenyl)ethyl]carbamate

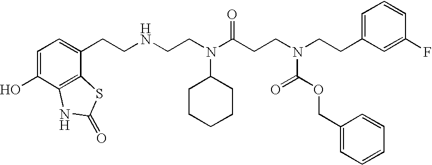

Part a) To a 500 mL vessel was charged para-toluenesulfonic acid monohydrate (76.23 g) and tetrahydrofuran (103.13 mL) was added. Benzyl {3-[cyclohexyl(2,2-dimethoxyethyl)amino]-3-oxopropyl}[2-(3-fluorophenyl)ethyl]carbamate (51.56 g, as a 61.9 wt % solution in 2-methyltetrahydrofuran) (as afforded by Preparation 2 (Form H) Step iv) was added. A line wash of tetrahydrofuran (154.69 mL) was added and the solution stirred to 20° C. After 1 h the reaction was cooled to <5° C. and then added to a previously prepared <5° C. solution of 10 M aqueous sodium hydroxide (50.3 mL) and sodium chloride (103.13 g) in water (469.2 mL) maintaining the internal temperature <15° C. A line wash of tetrahydrofuran (25.8 mL) was added then the mixture warmed to 20° C. The aqueous phase was discarded. The organic phase was transferred to a clean vessel and a tetrahydrofuran (25.8 mL) line rinse was applied. To this solution was added water (12.38 mL) and the solution retained for use in Part b).

Part b) To 7-(2-aminoethyl)-4-hydroxy-1,3-benzothiazol-2(3H)-one hydrobromide (32.09 g), sodium triacetoxyborohydride (44.71 g) was added tetrahydrofuran (206.25 mL), N-methylpyrrolidone (46.41 mL) and triethylamine (18.05 mL). The slurry was cooled to 10° C. The retained organic phase from Part a) was added over 32 min, then a line wash of tetrahydrofuran (25.8 mL) added. After 2 h, the mixture was warmed to 20° C. After 30 min, water (232.03 mL) and ethyl acetate (232.03 mL) were added. The aqueous phase was removed and the organic phase washed with 10 wt % aqueous sodium chloride (232.03 mL). The aqueous phase was removed. The organic phase was distilled under vacuum to ±25% of the original volume. Ethyl acetate (232.03 mL) was added and the mixture cooled to 20° C. The mixture was seeded with authentic material and the resulting suspension was stirred for 19 h. The suspension was filtered and the cake washed with ethyl acetate (232.03 mL) and dried under vacuum to yield the subtitled compound (37.521 g).

### vi) N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form H)

A portion of the material from Preparation 2 (Form H) Step v) (10.05 g) and acetic acid (35.18 mL) were added to a 250 mL jacketed vessel and the mixture was stirred at 25° C. for 10 min to give a homogeneous solution. A solution of hydrogen bromide in acetic acid (33 wt %; 30.15 mL) was added over 15-20 min using a pressure-equalizing dropping funnel/dip pipe assembly and the reaction is stirred for 2 h at 25° C. Methyl tert-butyl ether (16.08 mL) and ethanol (4.02 mL) were pre-mixed and added over ˜2 h and a ca. 4 mL sample of the bulk reaction mixture was withdrawn and charged with a 1 mg of seed of Preparation 2 (Form C). This mixture was shaken for ±3 min affording a large amount of precipitate. This suspension was returned to the bulk mixture, which was left to stir overnight at 25° C. under nitrogen. The solid product was then isolated by filtration under nitrogen and rinsed with methyl tert-butyl ether (20.10 mL). The material was oven dried in vacuo at 55° C. to yield the title compound (10.57 g). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form H) is presented in FIG. 6.

Preparation 2 (Form C) Alternative Procedure. N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihydrobromide salt (Form C). To a 500 mL jacketed vessel, equipped with an overhead stirrer, condenser, nitrogen inlet and temperature probes, was charged Preparation 2 (Form H) (15g). The vessel was charged with ethanol (300 mL) and stirred. The jacket was warmed to 85° C. and the mixture became a solution at 77.4° C. The mixture was stirred at reflux for 30 min, then the jacket was cooled from 85° C. to 5° C. at 0.5° C./min i.e. over 160 min. The product crystallised without seeding. The material was held at 5° C. for 2 h, then the material was isolated by filtration. The filtration was achieved on 70 mm Whatman type 54 paper and took 60 seconds. The cake was 11 mm high. A wash of Ethanol (Cake wash) (45 mL) was applied and this took 70 seconds to de-liquor. The material was collected and dried in a vacuum oven at 50° C. for 65 h. This afforded 11.96 g of the title compound. Analysis of the material by XRPD indicated it to be Form C.

Preparation 3 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt (Form A). A portion of the dihydrobromide from Preparation 2 was suspended in tetrahydrofuran and water (5:1), treated with a solution of saturated aqueous sodium bicarbonate (3 mol eq) in water, and the mixture stirred for 15 min. The tetrahydrofuran was removed in vacuo, sodium chloride was added, and the mixture was extracted with chloroform. The combined organic fractions were washed with water and brine, dried (anhydrous Na₂SO₄), filtered and treated with a solution of D-mandelic acid (3 mol eq) in acetonitrile (40 mL). The mixture was stirred for 2 h, filtered, washed with acetonitrile and dried to give the title compound as a colourless solid. ¹H NMR δ_{(DMSO)} 7.40 (4H, d), 7.38-7.14 (7H, m), 7.05 (3H, t), 6.82-6.67 (2H, m), 4.75-4.69 (2H, m), 4.10-3.97 (0.5H, m), 3.53-3.44 (0.5H, m), 3.35-3.22 (2H, m), 3.07-2.97 (4H, m), 2.92-2.73 (6H, m), 2.72-2.61 (4H, m), 1.78-1.69 (2H, m), 1.65-1.55 (2H, m), 1.52-1.17 (5H, m), 1.13-1.00 (1H, m) MS: APCI (+ve): 529 [M+H]⁺ The melting temperature of Preparation 3 (Form A) as determined by DSC was found to be 156° C. (onset, ±2° C.). Weight loss observed prior to melting by TGA was negligible. GVS determination gave 1% (±0.2%) weight increase (% w/w) at 80% RH. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt (Form A) is presented in FIG. 7.

Preparation 3 (Form A) Alternative Procedure. N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt (Form A). A portion of Preparation 2 (Form C) Alternative Procedure (30.00 g) and 2-MeTHF (120 mL) was charged to a jacketed vessel, ethanol (30 mL) was added and the resulting slurry was stirred at 20° C. In a separate vessel, potassium carbonate (18.01 g) was dissolved in water (240.00 mL) and the solution was equilibrated at 20° C., prior to addition to the 2-methyltetrahydrofuran/ethanol slurry, maintaining the internal temperature <25° C. The temperature was equilibrated at 20° C. with stirring and then the phases were allowed to settle. The lower aqueous phase was removed and discarded and the organic phase was filtered using a fines filter and the line was rinsed using ethanol (15 mL), again using a fines filter. (R)-(−)-Mandelic Acid (13.88 g) was dissolved in ethanol (90 mL) and the solution was charged via a fines filter to the vessel containing the free base. The line was rinsed into the vessel with ethanol (11.87 g) via a fines filter and further ethanol (270 mL) was added via a fines filter. The mixture was then seeded with authentic N-Cyclohexyl-N3-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt (30 mg) and then cooled to 0° C. After stirring overnight at this temperature, the mixture was filtered and the solid was washed with ethanol 60 mL), pre-filtered using a fines filter. The cake was pulled dry and the resulting material was dried under vacuum at 40° C. to constant weight to afford the title compound (27.917 g).

### Preparation 4 (Form A). (R)-1-[2-(4-Fluoro-phenyl)-ethyl]-3-((S)-2-phenyl-2-piperidin-1-yl-propionyloxy)-1-azonia-bicyclo[2.2.2]octane bromide (Form A)

### Intermediate A (Isomers 1 & 2): 2-Phenyl-2-piperidin-1-yl-propionic acid methyl ester

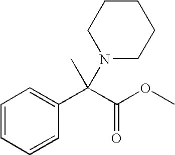

A solution of methyl 2-bromo-2-phenylpropanoate (1g) in acetonitrile (30 mL) was treated with piperidine (1 mL). The solution was stirred and heated under reflux for 3 h then concentrated to dryness. The residue was purified by flash column chromatography on silica gel using ether/isohexane (3:7) to afford the racemic sub-titled compound as a colourless oil (0.8 g). The mixture of enantiomers was separated by chiral hplc using a chiracel OJ-H column using an isocratic system of 80% isohexane/ethanol to afford the two enantiomers, which were defined as Isomer 1 and Isomer 2 in order of elution. 2-Phenyl-2-piperidin-1-yl-propionic acid methyl ester (Isomer 1) Chiral HPLC 80:20 isohexane:ethanol (isocratic). Chiracel OJ-H 4.6 mm×50 mm Retention time 1.09 min. ¹H NMR (400 MHz, CDCl₃) [symbol] 7.56-7.49 (2H, m), 7.35-7.20 (3H, m), 3.68 (3H, s), 2.54-2.45 (2H, m), 2.41-2.32 (2H, m), 1.64-1.54 (7H, m), 1.50-1.42 (2H, m). 2-Phenyl-2-piperidin-1-yl-propionic acid methyl ester (Isomer 2) Chiral HPLC 80:20 isohexane:ethanol (isocratic). Chiracel OJ-H 4.6 mm×50 mm Retention time 2.52 min. ¹H NMR (400 MHz, CDCl₃) [symbol] 7.56-7.49 (2H, m), 7.35-7.20 (3H, m), 3.68 (3H, s), 2.54-2.45 (2H, m), 2.41-2.32 (2H, m), 1.64-1.54 (7H, m), 1.50-1.42 (2H, m).

### Intermediate B: 2-Phenyl-2-piperidin-1-yl-propionic acid (R)-(1-aza-bicyclo[2.2.2]oct-3-yl) ester (Isomer 1).

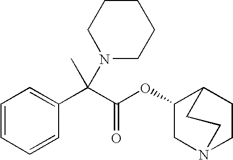

A mixture of 2-phenyl-2-piperidin-1-yl-propionic acid methyl ester (Intermediate A, Isomer 1) (0.9 g), (R)-quinuclidin-3-ol (1.157 g) and sodium hydride (60% in mineral oil, 0.335 g) in dry toluene (20 mL) was heated at 120° C. under an atmosphere of nitrogen for 8 h. The cooled reaction mixture was diluted with water (100 mL) and extracted with diethyl ether (2×150 mL). The combined extracts were dried (MgSO₄) and concentrated to give an oil. The crude product was purified by flash column chromatography on silica eluting with (ethyl acetate/methanol 9:1) to afford the titled compound (0.500g). ¹H NMR (400 MHz, DMSO) δ 7.59-7.51 (2H, m), 7.40-7.21 (3H, m), 4.72-4.62 (1H, m), 3.34-3.26 (1H, m), 3.04-2.92 (1H, m), 2.75-2.13 (7H, m), 1.89-1.75 (1H, m), 1.71-1.20 (14H, m).

### (R)-1-[2-(4-Fluoro-phenyl)-ethyl]-3-((S)-2-phenyl-2-piperidin-1-yl-propionyloxy)-1-azonia-bicyclo[2.2.2]octane bromide (Form A).

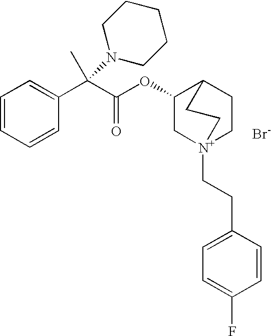

2-Phenyl-2-piperidin-1-yl-propionic acid (R)-(1-aza-bicyclo[2.2.2]oct-3-yl) ester (Intermediate B, Isomer 1) (3g) in acetonitrile (25 mL) was treated with 1-(2-bromoethyl)-4-fluorobenzene (2.384g) and the mixture stirred at RT for 24 h. The mixture was concentrated to dryness, and the residue purified on silica gel eluting with 10% methanol in dichloromethane. The product containing fractions were combined, concentrated to dryness and the foam residue re-dissolved in acetonitrile (20 mL). To the solution was added diethyl ether (40 mL) and the resulting solid collected by filtration. The solid was dissolved in hot acetone (75 mL) and then allowed to cool overnight. The resulting solid was collected by filration and dried at 50° C. to afford the titled compound (3.70 g). MS 465 [M]⁺¹H NMR (400 MHz, DMSO) δ 7.58-7.54 (2H, m), 7.40-7.32 (4H, m), 7.31-7.26 (1H, m), 7.23-7.16 (2H, m), 5.14-5.09 (1H, m), 3.95-3.85 (1H, m), 3.62-3.51 (1H, m), 3.50-3.36 (4H, m), 3.25-3.16 (2H, m), 2.95 (2H, t), 2.48-2.31 (4H, m), 2.24-2.18 (1H, m), 2.02-1.69 (4H, m), 1.57 (3H, s), 1.56-1.48 (4H, m), 1.47-1.40 (2H, m). Single crystal X-ray diffraction data obtained for Preparation 1 proved the structure to be (R)-1-[2-(4-Fluoro-phenyl)-ethyl]-3-((S)-2-phenyl-2-piperidin-1-yl-propionyloxy)-1-azonia-bicyclo[2.2.2]octane bromide. The data set was collected at RT with graphite monochromatizedo MoK(a) radiation on a KappaCCD Single-Crystal X-Ray diffractometer equipped with an k-axis goniometer and a CCD area detector (Nonius, 1998). The diffraction raw data were processed within the Denzo-SMN program package (Otwinowski & Minor, 1998) converting the information from the digital image frame to a file containing h, k, l indices, background and Lp corrected intensities of the diffraction spots, along with estimate of errors. On the basis of the crystal structure determined for Preparation 4, the absolute configuration of Intermediate A-Isomer 1 used has been assigned as (S)-2-Phenyl-2-piperidin-1-yl-propionic acid methyl ester. The melting temperature of Preparation 4 bromide Form A as determined by DSC gave found a double endothermic events occurring at 171° C. (1^{st} onset) and 183° C. (2^{nd} onset)(±2° C.). Weight loss observed prior to melting by TGA was negligible. GVS determination gave 0.1% weight increase (% w/w) at 80% RH (±0.2%). An XRPD spectrum of (R)-1-[2-(4-fluoro-phenyl)-ethyl]-3-((S)-2-phenyl-2-piperidin-1-yl-propionyloxy)-1-azonia-bicyclo[2.2.2]octane bromide (Form A) is presented in FIG. 8.

Preparation 4 Form C: (R)-1-[2-(4-Fluoro-phenyl)-ethyl]-3-((S)-2-phenyl-2-piperidin-1-yl-propionyloxy)-1-azonia-bicyclo[2.2.2]octane bromide (Form C). (R)-1-[2-(4-Fluoro-phenyl)-ethyl]-3-((S)-2-phenyl-2-piperidin-1-yl-propionyloxy)-1-azonia-bicyclo[2.2.2]octane bromide (above) (1g) was dissolved in methanol (5 mL) and the mixture warmed to 60° C. The mixture was allowed to cool to 40° C. whereupon solid started to form and the mixture was then re-heated to 50° C. Three 10 mL aliquots of methyl acetate were added to the mixture which was then allowed to slowly cool to room temperature and stirred for 18 h. The resulting solid was collected by filtration and then dried under reduced pressure at 50° C. to afford the titled compound (50 mg). ¹H NMR (400 MHz, DMSO) δ 7.51-7.60 (2H, m), 7.31-7.41 (4H, m), 7.25-7.31 (1H, m), 7.13-7.21 (2H, m), 5.08-5.15 (1H, m), 3.88-3.97 (1H, m), 3.53-3.63 (1H, m), 3.38-3.52 (4H, m), 3.15-3.26 (2H, m), 2.92-3.01 (2H, m), 2.31-2.48 (4H, m), 2.20-2.25 (1H, m), 1.72-2.04 (4H, m), 1.58 (3H, s), 1.48-1.56 (4H, m), 1.39-1.48 (2H, m). The melting temperature of Preparation 4 bromide Form C as determined by DSC was found to be 184° C. (onset) (±2° C.). Weight loss observed prior to melting by TGA was 4%. GVS determination gave 4% weight increase (% w/w) at 80% RH (±0.2%). An XRPD spectrum of (R)-1-[2-(4-fluoro-phenyl)-ethyl]-3-((S)-2-phenyl-2-piperidin-1-yl-propionyloxy)-1-azonia-bicyclo[2.2.2]octane bromide (Form C) is presented in FIG. 9.

Preparation 5. N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide The product from Preparation 2 (820 mg) was dissolved in aqueous THF and the solution was basified with saturated queous sodium bicarbonate solution and extracted into ethyl acetate (×3). The combined extracts were washed with brine, dried over Na₂SO₄, filtered and concentrated in vacuo to afford the title compound (600 mg).

Preparation 5 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide (Form A). A solution of a material from Preparation 5 (200 mg) in methanol (10 mL) was allowed to stand overnight. The resulting solid was isolated by filtration and dried. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-(3-alaninamide (Form A) is presented in FIG. 10.

Preparation 6 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Form A). A solution of a material from Preparation 5 (20 mg) in methanol (1 mL) was mixed thoroughly with a solution of naphthalene-2-sulfonic acid (70% w/w, 22.5 mg) in methanol (1 mL). The solvent was allowed to evaporate and the residue was slurried in IPA for 24 h. The resulting solid was isolated by filtration and dried to afford the title compound (21 mg). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Form A) is presented in FIG. 11.

Preparation 6 (Form B). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Form B). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged 2-naphthalenesulfonic acid hydrate (1.35 g) to give a homogeneous stirred solution. The mixture was diluted with ethanol (10 mL) and this did not afford a solid. On adding this solution to a stirred 1:1 solution of iso-hexane/diethyl ether (40 mL) which afforded a white solid which was collected and dried in a vacuum over at 40° C. to a constant weight of 2.41 g. The solid can be recrystallised from a mixture of ethanol (20 relative volumes i.e. 20 mL/g) and water (1.4 relative volumes i.e. 1.4 mL/g) to give material in form B. An XRPD spectrum of N-Cyclohexyl-N-³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di(naphthalene-2-sulfonic acid) salt (Form B) is presented in FIG. 12.

Preparation 7 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihippuric acid salt (Form A). A solution of a material from Preparation 5 (20 mg) in methanol (1 mL) was mixed thoroughly with a solution of hippuric acid (13.5 mg) in methanol (1 mL). The solvent was allowed to evaporate and the residue was slurried in IPA for 24 h. The resulting solid was isolated by filtration and dried to afford the title compound (2 mg). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dihippuric acid salt is presented in FIG. 13.

Preparation 8 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide sulfuric acid salt (Form A). A solution of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide, prepared by a similar method to Preparation 5 (10 mg) in ethanol (0.5 mL) was mixed thoroughly with a mixture of concentrated sulphuric acid (d=1.84 g mL⁻¹, 1 μL) in ethanol (1 mL) and the solvent was allowed to evaporate. The residue was triturated with acetonitrile and the solid was isolated by filtration and dried to give the title compound (12 mg). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide sulfuric acid salt (Form A) is presented in FIG. 14.

Preparation 9 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Form A). A solution of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide, prepared by a similar method to Preparation 5 (25 mg) in ethanol (1 mL) was mixed thoroughly with a solution of naphthalene-1,5-disulfonic acid (17 mg) in ethanol (1 mL) causing precipitation. The mixture was stirred overnight and the sold isolated by filtration, washed with ethanol and dried to give the title compound (34 mg). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Form A) is presented in FIG. 15.

Preparation 9 (Form B). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Form B). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture, which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged naphthalene-1,5-disulfonic acid tetrahydrate (1.06 g), which afforded a solution on stirring. After 20 min, ethanol (10 mL) was charged to the solution and on stirring the mixture afforded a white precipitate within 60 min. After a 60 hour stir out the material was isolated via filtration. The white solid was washed with ethanol (10 mL) and dried to constant weight of 2.17 g after 18 hrs in a vacuum oven at 40° C. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide naphthalene-1,5-disulfonic acid salt (Form B) is presented in FIG. 16.

Preparation 10 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form A). A solution of Preparation 5 (150 mg) in 1:1 tetrahydrofuran/acetonitrile (6 mL) was mixed with a solution of benzenesulfonic acid (90 mg) in acetonitrile (3 mL) and the mixture was allowed to stand in a stoppered flask overnight. The mixture was then concentrated in vacuo and the residue was azeotroped with acetonitrile (×3). Further acetonitrile was then added to the residue and the mixture was allowed to stand for 3 h, with occasional scratching to promote crystallization, resulting in precipitation of a solid. The resulting mixture was stirred for 18 h and the solid isolated by filtration and dried to afford the title compound (190 mg). An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form A) is presented in FIG. 17.

Preparation 10 (Form B). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form B). A sample of Preparation 10 (Form A) (10 mg) was partly dissolved in water (0.5 mL). The suspension was stirred continuously for 7 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form B) is presented in FIG. 18.

Preparation 10 (Form C). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form C). A sample of Preparation 10 (Form A) (10 mg) was partly dissolved in ethanol (0.5 mL). The suspension was stirred continuously for 7 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form C) is presented in FIG. 19.

Preparation 10 (Form D). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form D). A sample of Preparation 10 (Form A) (10 mg) was partly dissolved in iso-propanol (0.5 mL). The suspension was stirred continuously for 7 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form D) is presented in FIG. 20.

Preparation 10 (Form E). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form E). A sample of Preparation 10 (Form A) (10 mg) was partly dissolved in acetone (0.5 mL). The suspension was stirred continuously for 7 days at room temperature. The solid was separated using a centrifuge and material subsequently dried by a stream of air. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form E) is presented in

FIG. 21.

Preparation 10 (Form F). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form F). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged benzenesulfonic acid (934.9 mg) and on stirring this gave a homogeneous solution. After charging ethanol (10 mL) the mixture remained a solution at 23° C. No solid was observed after an overnight stir out at 23° C., nor when chilled back to −10° C. The mixture was poured into a vessel containing stirred tent-butyl methyl ether (40 mL). This gave a gummy oil, which on prolonged stirring turned over to a white solid. This was filtered and dried in a vacuum oven at 40° C. overnight to a constant weight of 2.31 g. A 0.5 g portion of was successfully recrystallised in ethanol (10 mL; i.e. 20 relative volumes i.e. 20 mL/g). This yielded a solution at reflux, which was cooled and filtered to provide the title compound (0.45 g) as a solid. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dibenzenesulfonic acid salt (Form F) is presented in FIG. 22.

Preparation 11 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide bis(4-methylbenzenesulfonic acid) salt (Form A). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged para-toluenesulfonic acid monohydrate (1.1 g). On stirring this gave a thick precipitate that was difficult to stir. The mixture was diluted with ethanol (10 mL) and the suspension stirred for 2 hours prior to filtration and washing with ethanol (10 mL). The white solid isolated was dried in a vacuum oven at 40° C. overnight to constant weight of 2.2 g. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide bis(4-methylbenzenesulfonic acid) salt (Form A) is presented in FIG. 23.

Preparation 12 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimethanesulfonic acid salt (Form A). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged methanesulfonic acid (380 μL ). This gave a bi-phasic solution which was homogenised by the addition of ethanol (10 mL). This solution was charged to a vessel containing stirred tert-butyl methyl ether (40 mL). This gave a gummy oil, which on prolonged standing turned over to a white solid. The solid was recovered by filtration and washed with tert-butyl methyl ether (20 mL). After drying to constant weight in a vacuum oven at 40° C. giving 0.956 g of a white solid. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimethanesulfonic acid salt (Form A) is presented in FIG. 24.

Preparation 13 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimaleic acid salt (Form A). A 2g portion of the dihydrobromide from Preparation 2 (Form C) Alternative Procedure was suspended in 2-methyltetrahydrofuran (8 mL) and ethanol (2 mL). To this suspension at 23° C. was charged a premixed solution of potassium carbonate (1.2 g) in water (16 mL). On stirring at 23° C. gave a clear bi-phasic mixture which after 90 min was poured into a separating funnel and the lower aqueous layer removed. The organic phase was made up to 10 mL total volume by addition of 2-methyltetrahydrofuran (0.7 mL) as a line wash into a clean vessel. To the stirred organic layer was then charged (Z)-2-butenedioic acid (686 mg) which on stirring gave a solution which afforded a white precipitate within 60 min. The mixture was diluted with ethanol (10 mL) to give a more readily stirrable suspension. After a 60 hour stir out the material was isolated via filtration. The white solid was washed with ethanol (10 mL) and dried to constant weight of 2.03 g after 18 hrs in a vacuum oven at 40° C. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide dimaleic acid salt (Form A) is presented in FIG. 25.

Preparation 14 (Form A). N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide disaccharin salt (Form A). A 5g portion of the dihydrobromide from Preparation 2 (Form C)—Alternative Procedure and saccharin sodium salt hydrate (3.31 g) was suspended in ethanol (75 mL) and water (1 mL). The mixture was held at reflux for 30 min. The mixture was then allowed to cool to 20° C. This resulted in a white suspension which was stirred for 66 hours. The solid was isolated via filtration and the solid washed with ethanol (10 mL). The solid was dried in a vacuum oven at 40° C. overnight to a constant weight of 4.86 g. An XRPD spectrum of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide disaccharin salt (Form A) is presented in FIG. 26.

### Preparation 15 (Form A). Dicyclohexylammonium N-[(Benzyloxy)carbonyl]-N-[2-(3-fluorophenyl)ethyl]-β-alaninoate (Form A)

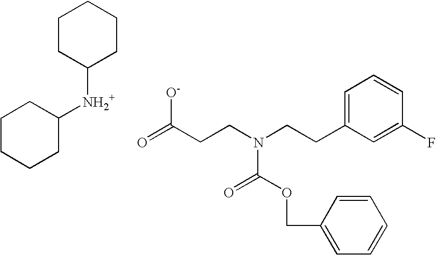

To a 250 mL three necked flask (fitted with an overhead stirrer and a thermometer) was charged a portion of the product of Preparation 2 Step iii) (10g) as a solution in TBME (88.4 mL of the assayed solution was required to give 10g input of N-[Benzyloxy)carbonyl]-N-[2-(3-fluorophenyl)ethyl]-β-alanine) The solution was stirred at 21° C. Dicyclohexylamine (5.76 mL) was weighed into a 50 mL conical flask and diluted with TBME (40 mL). The solution of dicyclohexylamine in TBME was added via a dropping funnel over ca. 10 min. After ±50% of the solution had been added, crystallisation started to occur on the walls of the vessel. After ±75% had been added, the mixture was thick with a white crystalline material and the internal temp had increased by 2° C. (21 to 23° C.). The mixture was stirred for ±120 min at 21° C. prior to filtration. The solid residue was disgarded and the filtrate was returned to the 250 mL vessel (washing in the small amount remaining solid off the walls) to give a stirred suspension which was cooled to 0° C. On cooling, more solid precipitated and the mixture was left stirring overnight, with slow warming to ±20° C. The mixture was diluted with a further 40 mL TBME and the resulting suspension was re-cooled to 0° C., and stirred at this temp for 30 min, prior to filtration. The solid residue was washed with TBME (2×20 mL) and the damp solid was dried in a vacuum oven at 33° C. for 2 h. The solid was dried until constant weight of 8.07 g as a white solid. ¹H NMR δ_{(DMSO)} 7.42-7.24 (6H, m), 7.09-6.92 (3H, m), 5.11-4.95 (2H, d), 3.49-3.41 (2H, t), 3.41-3.10 (2H, m), 2.85-2.74 (2H, m), 2.65-2.55 (2H, m), 2.41-2.30 (3H, m), 1.86-1.75 (4H, m), 1.71-1.60 (4H, dt), 1.59-1.50 (2H, dt), 1.29-0.93 (11H, m). An XRPD spectrum of Dicyclohexylammonium N-[[(Benzyloxy)carbonyl]-N-[2-(3-fluorophenyl)ethyl]-β-alaninoate (Form A) is presented in FIG. 27.

### Preparation 16. Synthesis of (R)-1-(4-fluorophenethyl)-3-((S)-2-phenyl-2-(piperidin-1-yl)propanoyloxy)-1-azoniabicyclo[2.2.2]octane 4-methylbenzenesulphonate

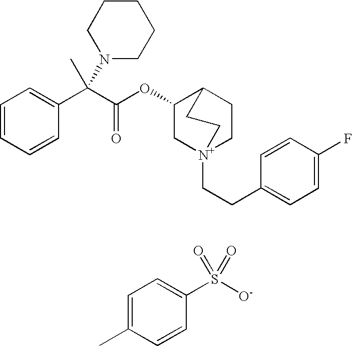

General Experimental Details for Preparations 16a and 16b.

Unless otherwise stated all reactions were carried out under an inert atmosphere: reagents and solvents were obtained commercially and used as received; reagent grade solvents were used. NMR spectra were measured on a Varian Unity Inova spectrometer at a proton frequency of 400 MHz. The MS spectra were measured on an Agilent 1100 MSD G1946D spectrometer.

Preparation 16a. (R)-1-(4-Fluorophenethyl)-3-((S)-2-phenyl-2-(piperidin-1-yl)propanoyloxy)-1-azoniabicyclo[2.2.2]octane bromide salt (0.6 g) prepared as described in WO2008/075005 (Example 44) was dissolved in dichloromethane (50 mL) and shaken with a solution of sodium 4-methylbenzenesulfonate (3.1 g) in water (100 mL), in three equal portions (˜33 mL). The organic layer was dried over anhydrous sodium sulphate, filtered and evaporated to dryness. The white foam was dissolved in hot acetoniltrile (˜5 mL) and allowed to cool to RT with stirring for 3 days. A white solid formed which was collected by filtration, washed with cold acetonitrile (˜2 mL) and dried in vacuo at 60° C. for 2 days to yield the product (0.490g). m/e M⁺465 ¹H NMR (400 MHz, _{d6}-DMSO) δ 7.55 (2H, d), 7.47 (2H, d), 7.40-7.31 (4H, m), 7.28 (1H, t), 7.19 (2H, t), 7.11 (2H, d), 5.1-5.08 (1H, m), 3.9-3.83 (1H, m), 3.59-3.50 (1H, m), 3.47-3.35 (4H, m), 3.25-3.14 (2H, m), 2.99-2.90 (2H, m), 2.47-2.31 (4H, m), 2.28 (3H, s), 2.24-2.18 (1H, m), 2.02-1.70 (4H, m), 1.57 (3H, s), 1.56-1.48 (4H, m), 1.48-1.38 (2H, m).

### Preparation 16b. Analytical HPLC and GC Conditions used in Steps a)-g)

Step a) was monitored by HPLC using an Ace phenyl column with standard aqueous/acetonitrile/TFA mobile phase on a gradient, with UV detection at 230 nm.

Steps b), c) and d) were monitored by GC using DB-5 capilllary column with FID detection and standard oven gradient from 40° C. to 300° C., with split injection.

Steps e), f), g) and h) are monitored by HPLC using C18 phase with standard aqueous/acetonitrile/TFA mobile phase on a gradient, with UV detection at 220 nm.

Step e) solvent composition was monitored by GC using a DB-624 capillary column with FID detection and oven gradient from 40° C. to 250° C., with split injection.

Step e) was monitored for levels of quinuclidinol by GC using an HP-1 capillary column with FID detection and oven gradient from 40° C. to 300° C., with split injection.

a) Methyl 2-phenylpropanoate. (+/−)-2-Phenylpropionic acid (20.5 g) was dissolved in methanol (62 mL) in a reaction vessel. Sulfuric acid (98%, 0.82 mL) was then charged followed by methanol (20.5 mL) as a line rinse. The reaction was then heated to 63° C. (±3° C.) and stirred at this temperature for up to 4 hrs. The reaction was monitored by HPLC analyzing the methyl 2-phenylpropanoate: (+/−)-2-phenylpropionic acid ratio (specification >97:3). Upon completion the reaction mixture was cooled to 23° C. (±3° C.). Cyclohexane (102 mL) was added followed by Na₂CO₃ (aq) (3.7% wt/wt, 61.5 mL). Layers were allowed to separate and the lower aqueous phase discarded. Water (61.5 mL) was then charged and the mixture stirred for 10 mins before the layers were separated discarding the lower aqueous phase. Cyclohexane (205 mL) was then charged to the organic phase. The reaction mixture was then distilled under reduced pressure at 45° C., 150-240 mbar removing 180 mL solvent. The reaction mixture was then cooled to 23° C. (±3° C.) yielding methyl 2-phenylpropanoate in a solution in cyclohexane.

b) Methyl 2-bromo-2-phenylpropanoate. Methyl 2-phenylpropanoate in a solution in cyclohexane (prepared in step a) (22.42 g; based on 100% yield from step a) was charged to a reaction vessel. Hydrobromic acid (48%, 0.62 mL) was then charged followed by cyclohexane (22.4 mL) as a line wash. Dibenzoyl peroxide (75%, 2.21 g) and N-bromosuccinimide (31.61 g) were then charged to the vessel and the reaction heated to 50° C. (±3° C.) and stirred at this temperature for at least 4 hrs. The reaction was monitored by GC analyzing the methyl 2-bromo-2-phenylpropanoate:methyl 2-phenylpropanoate ratio (specification >96:4). Upon completion the reaction mixture was cooled to 20° C. (±3° C.). The reaction mixture was filtered to remove the solid succinimide by-product, washing the filter cake twice with cyclohexane (22.4 mL). The solid by-product was discarded. NaHSO₃ (aq) (10% w/w, 81.9 mL1) was then charged and stirred for 15 mins before allowing the phases to separate discarding the lower aqueous phase. Water (81.9 mL,) was then charged and stirred for 15 mins before allowing the phases to separate discarding the lower aqueous phase. 3-Pentanone (201.9 mL) was then charged and the mixture was distilled at 45° C., 150-280 mbar removing 210 mL of solvent. The reaction mixture was cooled to 23° C. (±3° C.). 3-Pentanone (101 mL) was then charged and the solvent composition analyzed by GC (specification <30% cyclohexane) to yield methyl 2-bromo-2-phenylpropanoate in a solution of 3-pentanone.

c) Methyl 2-phenyl-2-piperidin-1-ylpropanoate. Methyl 2-bromo-2-phenylpropanoate in a solution of 3-pentanone (prepared in step b) (33.21 g; based on 100% yield from step b) was charged to a reaction vessel followed by piperidine (40.5 mL). The reaction was heated to 40° C. (±3° C.) and held for at least 4 hrs. The reaction was monitored by GC analyzing the methyl 2-phenyl-2-piperidin-1-ylpropanoate:methyl 2-bromo-2-phenylpropanoate ratio (specification >97:3). The reaction mixture was then cooled to 23° C. (±3° C.) and then filtered to remove the piperidine hydrobromide salt by-product, and the filter cake washed with methyl ^{t}butyl ether (66.4 mL). The filter cake was discarded. Methyl ^{t}butyl ether (133 mL) and hydrogen chloride (2.74M, 172.6 mL) were then added and the reaction mixture stirred for 15 mins before taking a pH reading to ensure pH<4. The layers were then allowed to separate retaining the lower aqueous phase. Hydrogen chloride (2.74M, 60.4 mL) was then added to the organic phase and the mixture stirred for at least 15 mins before allowing the phases to separate retaining the lower aqueous phase. The two aqueous phases were then combined, sampled and analyzed by GC to ensure all impurities were <0.5% with the exception of methyl 2-phenyl-3-(piperidin-1-yl)propanoate impurity). The aqueqous phase was then charged to a mixture of Na₂CO₃ (32.29 g), water (232 mL) and methyl ^{t}butyl ether (332 mL). The mixture was stirred for at least 15 mins before taking a pH reading to ensure pH>6. The layers were then allowed to separate discarding the lower aqueous phase. Water (66.4 mL) was then charged and stirred for 15 mins before allowing the phases to separate discarding the lower aqueous phase. Citric acid (0.8 wt %, 66.4 mL) was then charged to the organic phase and the mixture stirred for 15 mins before allowing the phases to separate discarding the lower aqueous phase. A second charge of citric acid (0.8 wt %, 66.4 mL) was then added to the organic phase and the mixture stirred for 15 mins before allowing the phases to separate discarding the lower aqueous phase. The organic phase was sampled and analyzed by GC to ensure methyl 2-phenyl-3-(piperidin-1-yl)propanoate impurity was less than 0.5%. The mixture was then distilled at 45° C., 80-220 mbar removing 265 mL solvent. Methanol (332 mL) was then charged to the vessel and the mixture again distilled at 45° C., 80-220 mbar removing 332 mL solvent. The reaction mixture was cooled to 23° C. (±3° C.) to yield methyl 2-phenyl-2-piperidin-1-ylpropanoate in a solution of methanol. The product was then analyzed by NMR assay and HPLC for purity. 23.8 g (at 100 w/w %) 70.5% yield, >99.5% HPLC purity.

d) (S)-methyl 2-phenyl-2-(piperidin-1-yl)propanoate. Racemic methyl 2-phenyl-2-piperidin-1-ylpropanoate (prepared in step c) was purified by Simulated Moving Bed (SMB) chromatography to yield methyl (S)-2-phenyl-2-piperidin-1-ylpropanoate. (S)-methyl 2-phenyl-2-(piperidin-1-yl)propanoate was isolated as a 40 w/w % solution in toluene. Typical conditions for the SMB purification were as follows:

| Stationary Phase | Chiralcel OJ 20 μm |
|---|---|
| Column bed dimensions (1 × d) | 25 cm × 11 cm |
| Mobile Phase | 20% Ethanol in iso-hexane |
| Feed Diluent | 20% Ethanol in iso-hexane |
| Feed Concentration (g/L) | 170 |
| Column Bed Pressure (Bar) | 50 |
| Injection Volume (ml) | 50 |
| Loading per injection (g) | 8.5 |
| Run time (min) | 14 |
| Flow (ml/min) | 500 |
| Wavelength (nm) | 230 |

e) (S)-((R)-quinuclidin-3-yl) 2-phenyl-2-(piperidin-1-yl)propanoate. (S)-methyl 2-phenyl-2-(piperidin-1-yl)propanoate (prepared in step d) (17.6 g as a 40 w/w % solution in toluene) was charged to a reaction vessel followed by (R)-(−)-3-quinuclidinol (9.5 g) and toluene (106 mL). The mixture was distilled at 60° C., 180-450 mbar removing 52 mL solvent. A sample was taken and analyzed by HPLC assay (specification 180-220 mg/mL (S)-methyl 2-phenyl-2-(piperidin-1-yl)propanoate. The reaction was then heated to 60° C. (±5° C.) and potassium tent-pentoxide (25 w/w %, 43.12 g) was added. The reaction mixture was stirred at 60° C. (±5° C.) for at least 2 hrs and monitored by HPLC analyzing the methyl (S)-methyl 2-phenyl-2-(piperidin-1-yl)propanoate:(S)-((R)-quinuclidin-3-yl) 2-phenyl-2-(piperidin-1-yl)propanoate ratio (specification >95:5) followed by toluene (8.8 mL) as a line rinse. The reaction mixture was cooled to 20° C. (±5° C.). Butanenitrile (88 mL) and water (88 mL) were charged and the mixture stirred for 20 mins before allowing the phases to separate discarding the lower aqueous phase. Water (88 mL) was charged and the mixture stirred for 20 mins before allowing the phases to separate discarding the lower aqueous phase. The organic phase was analysed by GC to ensure residual (R)-(−)-3-quinuclidinol levels were below 0.5%. The organic phase was distilled at 60° C., 100-430 mbar removing 142 mL of solvent. The reaction was then weighed and analysed by; NMR assay (w/w % of product) and GC (solvent composition) to determine the amount of product in solution and the solvent composition, toluene (18.5 mL, 1.05 vol) and butanenitrile (52.5 mL, 3 vol) was then added to the mixture to yield (S)-((R)-quinuclidin-3-yl) 2-phenyl-2-(piperidin-1-yl)propanoate (19.67 g, 81% yield) in a 7:3 butanenitrile:toluene solvent composition at 140 mg/mL concentration.

f) (R)-1-(4-Fluorophenethyl)-3-((S)-2-phenyl-2-(piperidin-1-yl)propanoyloxy)-1-azoniabicyclo[2.2.2]octane bromide. (S)-((R)-quinuclidin-3-yl) 2-phenyl-2-(piperidin-1-yl)propanoate (prepared in step e) (19.67 g as a 140 mg/mL solution in butanenitrile:toluene) was charged to a reaction vessel followed by 4-fluorophenethylbromide (13.99 g) and butanenitrile (19.7 mL). The reaction mixture was heated to 60° C. (±5° C.) and stirred at this temperature for at least 8 hrs. The reaction was monitored by HPLC analyzing the (S)-((R)-quinuclidin-3-yl) 2-phenyl-2-(piperidin-1-yl)propanoate:product ratio (specification >96:4). The reaction mixture was cooled to 40° C. over at least 40 mins (0.5° C./min) and then cooled to −5° C. over at least 6 hrs (0.125° C./min). During the cool no crystallisation had occurred when at 20° C. Therefore the reaction was seeded with a sample of (R)-1-(4-fluorophenethyl)-3-((S)-2-phenyl-2-(piperidin-1-yl)propanoyloxy)-1-azoniabicyclo[2.2.2]octane bromide (25 mg—obtainable by methods described in WO 2008/075005 Form A). After the reaction mixture reached −5° C. toluene (39.3 mL) was added and the slurry stirred at −5° C. for at least 1 hr. (R)-1-(4-Fluorophenethyl)-3-((S)-2-phenyl-2-(piperidin-1-yl)propanoyloxy)-1-azoniabicyclo[2.2.2]octane bromide was then collected by filtration, washing the filtercake with butanenitrile (39.3 mL). The (R)-1-(4-Fluorophenethyl)-3-((S)-2-phenyl-2-(piperidin-1-yl)propanoyloxy)-1-azoniabicyclo[2.2.2]octane bromide product was then dried under vacuum at 45° C. The product was then analysed by HPLC purity and NMR assay. 30g, 96% yield, >99.5% HPLC purity, >99.5 w/w % assay.

g) (R)-1-(4-Fluorophenethyl)-3-((S)-2-phenyl-2-(piperidin-1-yl)propanoyloxy)-1-azoniabicyclo[2.2.2]octane 4-methylbenzenesulphonate. A solution of sodium p-toluenesulfonate (26.97 g) in water (300 mL; 16.65 moles) was prepared. A 500 mL jacketed vessel was charged with (R)-1-(4-fluorophenethyl)-3-((S)-2-phenyl-2-(piperidin-1-yl)propanoyloxy)-1-azoniabicyclo[2.2.2]octane bromide (15.00 g). Butanenitrile (225 mL) and half of the sodium tosylate solution were added to the reaction vessel. The vessel was then stirred and heated to 35° C. When the vessel contents reached 35° C. and were adequately mixed the stirring was stopped and the phases allowed to settle. The lower aqueous phase was removed and discarded. The second half of the sodium tosylate solution was added and the vessel contents heated to 35° C. with stirring. When the vessel contents reached 35° C. and were adequately mixed the stirring was stopped and the phases allowed to settle. The lower aqueous phase was removed and discarded. Water (75 mL) was added and the mixture heated to 70° C. When the vessel contents reached 70° C. and were adequately mixed the stirring was stopped and the phases allowed to settle. The lower aqueous phase was removed and discarded. The hot organic phase was filtered into a clean vessel. The original vessel was washed with butanenitrile (30 mL) and this solvent was added to the filtrate via the filter into the clean vessel. The wet organic solution was distilled in order to azeodry it (120-150 mbar—vessel jacket at 80° C.). After ca. 60 mL of solvent had been distilled a precipitate was observed; contents were at 48° C. In total, 110 mL of solvent (10 mL water:100 mL butanenitrile) was collected. At this point the vacuum was released and the vessel contents warmed to 75° C. Acetonitrile (45 mL) was added and the vessel contents re-heated to 75° C. (not all material dissolved). More acetonitrile (45 mL) was added and the vessel contents re-heated to 75° C. (all material dissolved). The solution was cooled to 5° C. over 120 minutes (precipitation started at 65° C.). With the vessel contents at 5° C. the product was collected by filtration, washed with cold (5° C.) butanenitrile (30 mL) and pulled as dry as possible on the filter to give 15.27 g of solid. This solid was left open in a fume cupboard overnight to give (R)-1-(4-fluorophenethyl)-3-((S)-2-phenyl-2-(piperidin-1-yl)propanoyloxy)-1-azoniabicyclo[2.2.2]octane 4-methylbenzenesulphonate (15.22 g). The ratio of quaternary species to tosylate was determined as 1:1.01 by 400 MHz ¹HNMR using a 30 s relaxation delay.

h) (R)-1-(4-Fluorophenethyl)-3-((S)-2-phenyl-2-(piperidin-1-yl)propanoyloxy)-1-azoniabicyclo[2.2.2]octane 4-methylbenzenesulphonate (Recrystalisation). (R)-1-(4-Fluorophenethyl)-3-((S)-2-phenyl-2-(piperidin-1-yl)propanoyloxy)-1-azoniabicyclo[2.2.2]octane 4-methylbenzenesulphonate (7.50 g) and acetonitrile (90.00 mL) were charged to a vessel. The mixture was heated to 80° C. and the resulting solution held at 80° C. for 30 mins. The mixture was then cooled to 65° C. over 20 minutes. The solution was seeded with seed crystals of (R)-1-(4-fluorophenethyl)-3-((S)-2-phenyl-2-(piperidin-1-yl)propanoyloxy)-1-azoniabicyclo[2.2.2]octane 4-methylbenzenesulphonate (6 mg) and stirred at 65° C. for 1 hour. The reaction was then cooled to 5° C. over 10 hours and stirred at 5° C. for 6 hours. The solid product was then isolated by filtration, washing the filter cake with acetonitrile (15.00 mL). The product was then dried under vacuum at 45° C. to yield (R)-1-(4-fluorophenethyl)-3-((S)-2-phenyl-2-(piperidin-1-yl)propanoyloxy)-1-azoniabicyclo[2.2.2]octane 4-methylbenzenesulphonate as a white solid (6.6 g).

### Solid State Analysis of (R)-1-(4-fluorophenethyl)-3-((S)-2-phenyl-2-(piperidin-1-yl)propanoyloxy)-1-azoniabicyclo[2.2.2]octane 4-methylbenzenesulphonate

Instrument Details:
X-Ray Powder Diffraction (XRPD)—PANalytical X'Pert machine in 2Ø-Ø configuration or a PANalytical Cubix machine in Ø-Ø configuration over the scan range 2° to 40° 2Ø with 100-second exposure per 0.02° increment. The X-rays were generated by a copper long-fine focus tube operated at 45 kV and 40 mA. The wavelength of the copper X-rays was 1.5418 Å. The Data was collected on zero background holders on which ˜2 mg of the compound was placed. The holder was made from a single crystal of silicon, which had been cut along a non-diffracting plane and then polished on an optically flat finish. The X-rays incident upon this surface were negated by Bragg extinction.
Differential Scanning Calorimetry (DSC) thermograms were measured using a TA Q1000 Differential Scanning Calorimeter, with aluminium pans and pierced lids. The sample weights varied between 0.5 to 5 mg. The procedure was carried out under a flow of nitrogen gas (50 mL/min) and the temperature studied from 30 to 230° C. at a constant rate of temperature increase of 10° C. per minute.
Gravimetric Vapour Sorption (GVS) profiles were measured using a Surface Measurements Systems Dynamic Vapour Sorption DVS-1 or a DVS Advantage instrument. The solid sample ca. 1-5 mg was placed into a glass vessel and the weight of the sample was recorded during a dual cycle step method (40 to 90 to 0 to 90 to 0% relative humidity (RH), in steps of 10% RH).

A sample of material obtained by Preparation 16 as described herein above was analysed by XRPD (PANalytical X'Pert or Cubix system), GVS and DSC. The melting temperature as determined by DSC was found to be 189° C. (onset) (±2° C.). GVS determination gave 0.1% weight increase (% w/w) at 80% Relative Humidity (±0.2%). An XRPD spectrum of (R)-1-(4-Fluorophenethyl)-3-((S)-2-phenyl-2-(piperidin-1-yl)propanoyloxy)-1-azoniabicyclo[2.2.2]octane 4-methylbenzenesulphonate prepared according to Preparation 16 is presented in FIG. 28.

Adrenergic β Mediated cAMP Production.

Cell preparation. H292 cells were grown in 225 cm2 flasks incubator at 37° C., 5% CO₂ in RPMI medium containing, 10% (v/v) FBS (foetal bovine serum) and 2 mM L-glutamine.

Experimental Method. Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 0.05×10⁶ cells per mL. 5000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time Compound Z was added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP detection. The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. A concentration response curve for Compound Z was constructed and data was fitted to a four parameter logistic equation to determine both the pEC₅₀ and Intrinsic Activity. Intrinsic Activity was expressed as a fraction relative to the maximum activity determined for formoterol in each experiment. A result for Compound Z is in Table 1.

Selectivity Assays.

Adrenergic α1D.

Membrane Preparation. Membranes were prepared from human embryonic kidney 293 (HEK293) cells expressing recombinant human alp receptor. These were diluted in Assay Buffer (50 mM HEPES, 1 mM EDTA, 0.1% gelatin, pH 7.4) to provide a final concentration of membranes that gave a clear window between maximum and minimum specific binding.

Experimental Method.

Assays were performed in U-bottomed 96-well polypropylene plates. 10 mL [³H]-prazosin (0.3 nM final concentration) and 10 μL of Compound Z (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Result is shown in Table 1 below.

Adrenergic β1.

Membrane Preparation. Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

Experimental Method.

Assays were performed in U-bottomed 96-well polypropylene plates. 10 μL [¹²⁵I]-Iodocyanopindolol (0.036 nM final concentration) and 10 ΞL of Compound Z (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding). A result is shown in Table 1 below.

Dopamine D2.

Membrane Preparation. Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

Experimental Method.

Assays were performed in U-bottomed 96-well polypropylene plates. 30 mL [³H]-spiperone (0.16 nM final concentration) and 30 μL of Compound Z (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding). A result for Compound Z is shown in Table 1.

TABLE 1

| Preparation | β2 | β2 | α1 bind | β1 bind | D2 bind |
|---|---|---|---|---|---|
| No. | pEC50 | Int Act | pIC50 | pIC50 | pIC50 |
| 2 | 8.6 | 0.85 | 6.7 | 5.2 | 6.4 |

The present invention will now be further explained by reference to the following illustrative Examples.

### EXAMPLE 1

### Evaluation of Compound Activity on Intra-Alveolar Neutrophil Migration after Aerosol Challenge with Lipopolysacchamide (LPS) in the CRL:CD Rat.

LPS challenge in CRL:CD rats causes an influx of inflammatory cells into the lungs. Rats are challenged either with an aerosol of 0.9% w/v saline or 0.1 mg/mL LPS in 0.9% saline for 30 min or an intratracheal dose of 0.1-10 m/kg. This is repeated up to 8 times according to the experimental protocol. Rats are dosed with vehicle, standard compound or test compound by the appropriate route and frequency at various time points before and after challenge depending upon the experimental protocol. Test compound groups could either be the same compound at different doses or single doses of different compounds or a combination of the two. Test compounds are given by intraperitoneal, intravenous or subcutaneous injection or by inhalation or intratracheal administration.

The rats are euthanized at various time points after challenge depending upon the nature of the study, but typically 4 hr after LPS challenge with 1 mL pentobarbitone sodium. A tracheotomy is performed and a cannula inserted. The airway is then lavaged using 3 mL sterile PBS at room temperature. The PBS is left in the airway for 10 seconds before being removed. The PBS containing cells is placed into a 15 mL centrifuge tube on ice. This process is repeated three times.

An aliquot of BAL fluid is removed and counted on Sysmex (Sysmex UK, Milton Keynes). Cytospin slides are prepared by adding a 100 μl aliquot of BAL fluid into cytospin funnels in a Shandon Cytospin3 operated at 700 rpm for 5 min. Slides are stained on the Hema-Tek-2000 automatic slide stainer, using Wright-Giemsa stain and typically, 200 cells are counted under a microscope. Cells are classified as eosinophils, neutrophils and mononuclear cells (mononuclear cells included monocytes, macrophages and lymphocytes) and are expressed as a percentage of the total count.

### EXAMPLE 2

### Evaluation of Compound Activity on Intra-Alveolar Neutrophil Migration after Aerosol Challenge with Lipopolysacchamide (LPS) in the Guinea-Pig

Male Dunkin-Hartley guinea-pigs (300-600g) are placed into open fronted guinea-pig holding cones attached at random around a cylindrical aerosol chamber. Guinea-pigs are held in the challenge cones and exposed to an aerosol of vehicle, or LPS at concentrations of 0.1-30 m/ml in 0.9% saline per group Aerosols are generated using 2 jet nebulisers per column with a flow rate of 12 L/m. 10 ml of the challenge agent is placed into each nebuliser. Alternatively animals receive an intratracheal dose of 0.1-10 μg/kg. This is repeated up to 8 times according to the experimental protocol.

Guinea-pigs are dosed with vehicle, standard compound or test compound by the appropriate route and frequency at various time points before and after challenge depending upon the experimental protocol. Test compound groups could either be the same compound at different doses or single doses of different compounds or a combination of the two. Test compounds are given by intraperitoneal, intravenous or subcutaneous injection or by inhalation or intratracheal administration. Challenged guinea-pigs are killed by anaesthesia overdose (0.5 ml Euthetal i.p.) at 4 h-24 h post challenge. The lungs are then lavaged. After the trachea is exposed and cannulated using a luer fitting cannula (orange=size 8FG), the lungs are lavaged with 3×5 ml aliquots of Hanks Buffered Salt Solution (HBSS, EDTA-free). The lavaging is performed with gentle massaging of the chest to ensure appropriate agitation of the fluid in the lungs. The washes are harvested into a 15 ml conical, polypropylene centrifuge tube, an aliquot of BAL fluid is removed and counted on Sysmex (Sysmex UK, Milton Keynes). Cytospin slides are prepared by adding a 100 μl aliquot of BAL fluid into cytospin funnels in a Shandon Cytospin3 operated at 700 rpm for 5 min. Slides are stained on the Hema-Tek-2000 automatic slide stainer, using Wright-Giemsa stain and typically, 200 cells are counted under a microscope. Cells are classified as eosinophils, neutrophils and mononuclear cells (mononuclear cells included monocytes, macrophages and lymphocytes) and are expressed as a percentage of the total count.

### EXAMPLE 3

### Evaluation of Compound Activity on Intra-Alveolar Neutrophil Migration after Aerosol Challenge with Lipopolysacchamide (LPS) in the Mouse

Male C57BL/6/J or BALB/C mice (20-35g) are placed in Perspex exposure boxes in groups of up to 20 and exposed to an aerosol of either 0.3 mg/ml LPS or 0.9% w/v saline. The LPS (Sigma, E. Coli, Ref L-3755, Serotype 026:B6, Lot no. 111k4078) is made up in 0.9% w/v saline. An aerosol is generated using two jet nebulisers operated at a flow rate of 12 L/min (6 L/min for each nebuliser) for 15 min. Alternatively animals receive an intratracheal dose of 0.1-10 μg/kg. This may be repeated up to 8 times.

Mice are dosed with vehicle, standard compound or test compound by the appropriate route and frequency at various time points before and after challenge depending upon the experimental protocol. Test compound groups could either be the same compound at different doses or single doses of different compounds or a combination of the two. Test compounds are given by intraperitoneal, intravenous or subcutaneous injection or by inhalation or intratracheal administration.

Mice are killed with an overdose of Euthatal i.p 30 minutes, 1-24 hr after LPS challenge. When circulation has ceased, the trachea is cannulated (Portex intravenous cannula) and the airways lavaged with 3×0.3 ml of Isoton II (Beckman Coulter Ref. 8448011 Lot no. 25775). For cytospins, 100 ρl of the BALF is added to a cytospin funnel and spun, using a ThermoShandon Cytospin model 3 or 4, at 700 rpm for 5 min. Cells on the slide are stained on the Hema-Tek-2000 automatic slide stainer, using Wright-Giemsa stain and differential cell counts carried out to differentiate eosinophils, neutrophils and lymphomononuclear cells (including monocytes, macrophages and lymphocytes). Typically, 200 cells are counted per slide and each cell type expressed as a percentage of the total count. BALF total white cell count is measured using a Sysmex (Sysmex UK, Milton Keynes).

### EXAMPLE 4

### Evaluation of Lung Function in Anaesthetised Guinea-Pigs

Male Dunkin-Hartley guinea-pigs (300-600g) are weighed and dosed with either vehicle or compound in an appropriate vehicle according to the experimental protocol via the intratracheal route under recoverable gaseous anaesthesia (5% halothane in oxygen). Following dosing, the animals are administered supplemental oxygen and monitored until full recovery. Typically a dose volume of 0.5 mL/kg is used for the intratracheal route. In a dose response study, animals are dosed with compound or vehicle two hours prior to the administration of histamine. Test compound groups could either be the same compound at different doses or single doses of different compounds or a combination of the two.

The guinea-pigs are anaesthetised with pentobarbitone (1 mL/kg of 60 mg/mL solution intraperitoneally) approximately 30 minutes prior to the first bronchoconstrictor administration. The trachea is cannulated (Portex intravenous cannula, 200/300/070 (orange) or 200/300/060 (yellow)) and the animal ventilated using a constant volume respiratory pump (Harvard Rodent Ventilator model 683) at a rate of 60 breath/min and a tidal volume of 5 ml/kg. A jugular vein is cannulated (Portex intravenous catheter 200/300/010 (green)) for the administration of histamine or maintenance anaesthetic (0.1 mL of pentobarbitone solution, 60 mg/mL, as required).

The animals are then transferred to a Flexivent System (SCIREQ, Montreal, Canada) in order to measure airway resistance. The animals are ventilated (quasi-sinusoidal ventilation pattern) at 60 breaths/min at a tidal volume of 5 mL/kg. A positive end expiratory pressure of 2-3 cmH₂O is applied. Respiratory resistance is measured using the Flexivent “snapshot” facility (1 second duration, 1 Hz frequency). Once stable baseline resistance value has been obtained the animals are given histamine dihydrochloride or methacholine in ascending doses (Histamine; 0.5, 1, 2, 3 and 5 μg/kg, i.v., methacholine; 3, 10 and 30 μg/kg, i.v.) at approximately 4-minute intervals via the jugular catheter. After each administration of histamine the peak resistance value is recoreded. Guinea pigs are euthanised with approximately 1.0 mL pentobarbitone sodium (Euthatal) intravenously after the completion of the lung function measurements.

Percentage bronchoprotection produced by a compound is calculated at each dose of histamine as follows:

$$\% bronchoprotection = \frac{\% {changeR}_{veh} - \% {changeR}_{cmpd}}{\% {changeR}_{veh}}$$

Where % change Rᵥₑₕ is the mean of the maximum percentage change in airway resistance in the vehicle treated group.

### EXAMPLE 5

### Evaluation of Compounds on Antigen Induced Eosinophilia in Ovalbumin Sensitised Brown Norway Rats

On day 0 of the study Brown Norway rats are given a subcutaneous injection of 500 μg ovalbumin adsorbed onto 100 mg aluminium hydroxide in 0.4 mL saline in two distinct sites, approximately 0.2 mL per site. Day 14 and 15 following sensitisation the rats are challenged with aerosolised ovalbumin for 15 minutes. The rats are placed in groups of 10 in an acrylic box (internal dimensions 320 mm wide×320 mm deep×195 mm high, 20 L volume). 8 mL of 10 mg/mL ovalbumin in 0.9% saline, or 0.9% saline alone, is placed in each of two jet nebulizers (Sidestream®, Profile Respiratory Systems Ltd.). Compressed air at 6 L/min is passed through each nebulizer and the output of the nebulizers is passed into the box containing the rats.

Rats are dosed via the appropriate route with vehicle, standard compound or test compound at various time points before and after challenge depending upon the experimental protocol. Rats are euthanised with 0.5 mL pentobarbitone sodium (Euthatal) intraperitoneally at various times after challenge. A tracheotomy is performed and the trachea cannulated. The airway is then lavaged using 3 mL sterile PBS at room temperature. The PBS is left in the airway for 10 seconds before being removed. The PBS containing cells is placed into a 15 mL centrifuge tube on ice. This process is repeated three times. The final volume recovered is recorded. An aliquot of BAL fluid is removed and counted using a Sysmex (Sysmex UK, Milton Keynes).

Cytospin slides are prepared by adding a 100 μl aliquot of BAL fluid into cytospin funnels in a Shandon Cytospin 3 operated at 700 rpm for 5 min. Slides are stained on the Hema-Tek-2000 automatic slide stainer, using Wright-Giemsa stain and typically, 200 cells are counted under a microscope. Cells are classified as eosinophils, neutrophils and mononuclear cells. Mononuclear cells included monocytes, macrophages and lymphocytes.

### EXAMPLE 6

### Evaluation of Compounds on Antigen Induced Eosinophilia in Ovalbumin Sensitised Mice

20-25g male BALB/c mice are sensitized to ovalbumin by i.p administration of 100 μg of grade V ovalbumin (Sigma) adsorbed onto 1 mg of aluminium hydroxide gel mixture (Fisher Scientific UK) in 0.3 ml saline. Groups of mice are pre-dosed with compound If required, a minimum of two weeks after sensitization. They are then dosed daily for 1-8 days as study protocol specified, with test compound or 0.25 ml vehicle.

Each day of the 1-8 days, 1 hour after dosing, the mice are placed in perspex chambers (20×11×11 cm, 10 mice max./chamber) and administered an aerosol challenge of 20 mg ml⁻¹ ovalbumin for 36 min (8 ml for 18 min followed by another 8 ml for 18 min). Aerosol delivery is achieved using a DeVilbiss jet nebulizer with a flow rate of 61 min⁻¹. 24 h after the last dose the mice are killed with euthatal 0.2 ml i.p. and blood samples are taken (in EDTA tubes) for differential cell count analysis, the trachea is cannulated using a pink luer mount Portex cannula cut to 1 cm and the lungs are lavaged using 3 washes of 1 ml of Isoton II. For cytospins, 100 μl of the BALF is added to a cytospin funnel and spun, using a ThermoShandon Cytospin model 3 or 4, at 700 rpm for 5 min. Cells on the slide are stained on the Hema-Tek-2000 automatic slide stainer, using Wright-Giemsa stain and differential cell counts carried out to differentiate eosinophils, neutrophils and lymphomononuclear cells (including monocytes, macrophages and lymphocytes). Typically, 200 cells are counted per slide and each cell type expressed as a percentage of the total count. BALF total white cell count is measured using a Sysmex (Sysmex UK, Milton Keynes).

### EXAMPLE 7

### Evaluation on the Effect of Compound on Lung Function and BAL-Neutrophilia Following Acute Smoke Exposure in the Mouse

BALB/c or C57BL6/J mice undergo whole body exposure to main stream smoke (50 min/12 cigarettes) and fresh air once or twice a day for 1-9 days. Mice are dosed via the appropriate route with vehicle, standard compound or test compound at various time points before and after challenge depending upon the experimental protocol. On the final day of the experiment, mice are either killed with euthatal 0.2 ml i.p. and broncho-aveolar lavage fluid obtained for analysis of white blood cell infiltration (as described above) or lung function is assessed using a Flexivent System (SCIREQ, Montreal, Canada). Alternatively lung mechanics are measured using a forced manoeuvres system (EMMS).

Mice are anaesthetised with pentobarbitone ( 1/10dilution at a dose volume of 1 mL/kg intraperitoneally). The trachea is cannulated and the animal transferred to the Flexivent System where they are ventilated (quasi-sinusoidal ventilation pattern) at a rate of 150 breath/min and a tidal volume of 10 ml/kg in order to measure airways resistance.

Respiratory resistance is measured using the Flexivent “snapshot” facility (1 second duration, 1 Hz frequency). Mice are euthanised with approximately 0.5 mL pentobarbitone sodium (Euthatal) intravenously after the completion of the lung function measurements.

### EXAMPLE 8

### Evaluation of Bronchodilator Activity in the Guinea Pig Isolated Tracheal Ring Preparation

In this Example:

Compound W is: N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide di-D-Mandelate salt;

Compound X is: [2-(4-Chloro-benzyloxy)-ethyl]-[2-((R)-Cyclohexyl-hydroxy-phenyl-methyl)-oxazol-5-ylmethyl]-dimethyl-ammonium hemi-napadisylate salt (see WO 2008/096136); and,

Compound Y is: (R)-1-[2-(4-Fluoro-phenyl)-ethyl]-3-((S)-2-phenyl-2-piperidin-1-yl-propionyloxy)-1-azonia-bicyclo[2.2.2]octane bromide

Guinea pigs (300-500 g) were killed by cervical dislocation and the trachea was isolated. The trachea was cut into segments 2-3 cartilage rings in width and suspended in 10 ml organ baths in modified Krebs' solution (mM; NaCl, 90; NaHCO₃, 45; KCl, 5; MgSO₄.7H₂O, 0.5; Na₂HPO₄.2H₂O, 1; CaCl₂, 2.25; glucose, 10; pH 7.4 gassed with 5% CO₂, 95% O₂ at 37° C.). The tracheal rings were attached to an isometric force transducer for the measurement of isometric tension. The tissues were washed and a force of 1 g was applied to each tissue. The rings were contracted with methacholine (1 μM). Once the contraction had reached a plateau, vehicle (0.01% DMSO in distilled H₂O), compound W (1 nM or 3 nM), compound X (1 nM), compound Y (1 nM), a combination of compound W (3 nM) and compound X (1 nM), or a combination of compound W (1 nM) and compound Y (1 nM) was added and the tissue left for 60 min. The tension was measured in each ring at 60 min following compound addition and was expressed as a % relaxation of the constriction to methacholine (1 μM) (mean±s.e.mean). Data were collected using the Chart 4 software (ADInstruments, Charlgrove, UK).

Assessment of the combination of compound W and compound X: The dilator response (expressed as a percentage of the maximum response to methacholine (1 μM)) to compound W (3 nM) was 46±10.7, the percentage relaxation to compound X (1 nM) was 1±0.5 and the percentage relaxation to a combination of compound W (3 nM) and compound X (1 nM) was 65±15.7. The percentage relaxation to vehicle was 6±4.6 (n=4; FIG. 29).

Assessment of the combination of compound W and compound Y: The dilator response (expressed as a percentage of the maximum response to methacholine (1 μM)) to compound W (1 nM) was 48±3.9, the percentage relaxation to compound Y (1 nM) was 17±3.4 and the percentage relaxation to a combination of compound W (1 nM) and compound Y (1 nM) was 63±5.4. The percentage relaxation to vehicle was 3±2.5 (n=6; FIG. 30).

1. A pharmaceutical product, wherein:
the product comprises:
a first active ingredient which is N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof; and
a second active ingredient selected from:
a non-steroidal Glucocorticoid Receptor (GR Receptor) Agonist;
an antioxidant;
a CCR1 antagonist;
a chemokine antagonist (not CCR1);
a corticosteroid;
a CRTh2 antagonist;
a DP1 antagonist;
an Histone Deacetylase Inducer;
an IKK2 inhibitor;
a COX inhibitor;
a lipoxygenase inhibitor;
a leukotriene receptor antagonist;
an MPO inhibitor;
a muscarinic antagonist;
a p38 inhibitor;
a PDE inhibitor;
a PPARγ agonist;
a protease inhibitor;
a Statin;
a thromboxane antagonist;
a vasodilator; and
an ENAC blocker (Epithelial Sodium-channel blocker);
the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or
(R)-1-[(2-Methyl-pyridin-4-ylcarbamoyl)-methyl]-3-(1-phenyl-cycloheptanecarbonyloxy)-1-azonia-bicyclo[2.2.2]octane X; and
X is a pharmaceutically acceptable anion of a mono or polyvalent acid.

2. A pharmaceutical product as claimed in claim 1, wherein the first active ingredient is a salt of hydrochloride, hydrobromide, trifluoroacetate, sulphate, phosphate, acetate, fumarate, maleate, tartrate, lactate, citrate, pyruvate, succinate, oxalate, methanesulphonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate, edisylate, isethionate, 2-mesitylenesulphonate, 2-naphthalenesulphonate, 2,5-dichlorobenzenesulphonate, D-mandelate, L-mandelate, cinnamate, benzoate, adipate, esylate, malonate, mesitylate, napsylate, camsylate, formate, glutamate, glutarate, glycolate, hippurate, orotate, xylate, pamoic, palmitate, or furoate.

3. A pharmaceutical product as claimed in claim 1, wherein the first active ingredient is in the form of a di-D-mandelate salt.

4. A pharmaceutical product as claimed in claim 1, wherein:
the second active ingredient is:
a non-steroidal Glucocorticoid Receptor (GR Receptor) Agonist;
a CCR1 antagonist;
a chemokine antagonist (not CCR1);
a corticosteroid;
an IKK2 inhibitor;
a muscarinic antagonist;
a p38 inhibitor; or
a PDE inhibitor;
the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or
(R)-1-[(2-Methyl-pyridin-4-ylcarbamoyl)-methyl]-3-(1-phenyl-cycloheptanecarbonyloxy)-1-azonia-bicyclo[2.2.2]octane X; and
X is a pharmaceutically acceptable anion of a mono or polyvalent acid.

5. A pharmaceutical product, wherein the product comprises:
N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof, and
Tiotropium bromide.

6. A pharmaceutical product, wherein the product comprises:
N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof, and
Glycopyrrolate.

7. A pharmaceutical product, wherein the product comprises:
N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a pharmaceutically acceptable salt thereof, wherein:
the counter-ion of the salt is chloride, bromide, sulfate, methanesulfonate, benzenesulfonate (besylate), toluenesulfonate (tosylate), napthalenebissulfonate (napadisylate), phosphate, acetate, citrate, lactate, tartrate, mesylate, maleate, fumarate, or succinate; and
(R)-1-[2-(4-Fluoro-phenyl)-ethyl]-3-((S)-2-phenyl-2-piperidin-1-yl-propionyloxy)-1-azonia-bicyclo[2.2.2]octane.

8-10. (canceled)

11. A method of treating a respiratory disease, wherein:
the method comprises simultaneously, sequentially, or separately administering to a patient in need thereof:
(a) a therapeutically effective dose of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof; and
(b) a therapeutically effective dose of a non-steroidal Glucocorticoid Receptor (GR Receptor) Agonist; an antioxidant; a CCR1 antagonist; a chemokine antagonist (not CCR1); a corticosteroid; a CRTh2 antagonist; a DP1 antagonist; an Histone Deacetylase Inducer; an IKK2 inhibitor; a COX inhibitor; a lipoxygenase inhibitor; a leukotriene receptor antagonist; an MPO inhibitor; a muscarinic antagonist; a p38 inhibitor; a PDE inhibitor; a PPARγ agonist; a protease inhibitor; a Statin; a thromboxane antagonist; a vasodilator; or an ENAC blocker (Epithelial Sodium-channel blocker);
the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or
(R)-1-[(2-Methyl-pyridin-4-ylcarbamoyl)-methyl]-3-(1-phenyl-cycloheptanecarbonyloxy)-1-azonia-bicyclo[2.2.2]octane X; and
X is a pharmaceutically acceptable anion of a mono or polyvalent acid.

12. A kit, wherein the kit comprises:
a preparation of a first active ingredient which is as defined in claim 1, and
a preparation of a second active ingredient which is as defined in claim 1.

13. A pharmaceutical composition, wherein:
the composition comprises, in admixture:
N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino}ethyl)-β-alaninamide or a salt thereof, and
a non-steroidal Glucocorticoid Receptor (GR Receptor) Agonist; an antioxidant; a CCR1 antagonist; a chemokine antagonist (not CCR1); a corticosteroid; a CRTh2 antagonist; a DP1 antagonist; an Histone Deacetylase Inducer; an IKK2 inhibitor; a COX inhibitor; a lipoxygenase inhibitor; a leukotriene receptor antagonist; an MPO inhibitor; a muscarinic antagonist; a p38 inhibitor; a PDE inhibitor; a PPARγ agonist; a protease inhibitor; a Statin; a thromboxane antagonist; a vasodilator; or an ENAC blocker (Epithelial Sodium-channel blocker);
the muscarinic antagonist is not:
(R)-3-(1-Phenyl-cycloheptanecarbonyloxy)-1-(pyrazin-2-ylcarbamoylmethyl)-1-azonia-bicyclo[2.2.2]octane X;
(R)-3-(1-Phenyl-cycloheptanecarbonyloxy)-1-(pyridazin-3-ylcarbamoylmethyl)-1-azonia-bicyclo[2.2.2]octane X;
(R)-3-[1-(3-Fluoro-phenyl)-cycloheptanecarbonyloxy]-1-(pyrazin-2-ylcarbamoylmethyl)-1-azonia-bicyclo[2.2.2]octane X;
(R)-3-[1-(3-Fluoro-phenyl)-cycloheptanecarbonyloxy]-1-(isoxazol-3-ylcarbamoylmethyl)-1-azonia-bicyclo[2.2.2]octane X;
(R)-3-(1-Phenyl-cycloheptanecarbonyloxy)-1-(pyridin-2-ylcarbamoylmethyl)-1-azonia-bicyclo[2.2.2]octane X;
(R)-1-[(5-Fluoro-pyridin-2-ylcarbamoyl)-methyl]-3-(1-phenyl-cycloheptanecarbonyloxy)-1-azonia-bicyclo[2.2.2]octane X;
(R)-3-(1-Phenyl-cycloheptanecarbonyloxy)-1-(pyridin-3-ylcarbamoylmethyl)-1-azonia-bicyclo[2.2.2]octane X; or
(R)-1-[(2-Methyl-pyridin-4-ylcarbamoyl)-methyl]-3-(1-phenyl-cycloheptanecarbonyloxy)-1-azonia-bicyclo[2.2.2]octane X; and
X is a pharmaceutically acceptable anion of a mono or polyvalent acid.

14. A pharmaceutically acceptable salt of N-Cyclohexyl-N³-[2-(3-fluorophenyl)ethyl]-N-(2-{[2-(4-hydroxy-2-oxo-2,3-dihydro-1,3-benzothiazol-7-yl)ethyl]amino 1 ethyl)-β-alaninamide, wherein:
the salt is formed with naphthalene-2-sulfonic acid, hippuric acid, sulfuric acid, 4-methylbenzenesulfonic acid, naphthalene-1,5-disulfonic acid, benzenesulfonic acid, methanesulfonic acid, maleic acid, or saccharin.

15. A pharmaceutical composition, wherein the composition comprises:
a salt as claimed in claim 14, and
a pharmaceutically acceptable adjuvant, diluent, or carrier.

16-18. (canceled)

19. A method of treating, or reducing the risk of, a respiratory disease or condition which comprises administering to a patient in need thereof a therapeutically effective amount of a salt as claimed in claim 14.

20. A compound, wherein the compound is dicyclohexylammonium N-[(benzyloxy)carbonyl]-N-[2-(3-fluorophenyl)ethyl]-β-alaninoate:

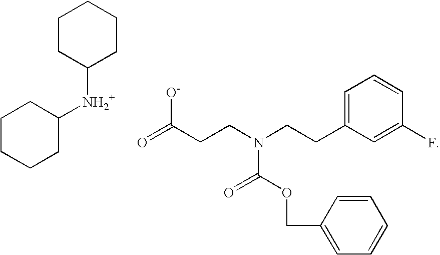

21. A kit of claim 12, wherein the kit further comprises instructions for simultaneously, sequentially, or separately administering the preparations to a patient in need thereof.
